(19) United States
(12) Patent Application Publication
(10) Pub. No.: US 2023/0332226 A1
(43) Pub. Date: Oct. 19, 2023

(54) COMPOSITIONS FOR SURFACE AMPLIFICATION AND USES THEREOF

(71) Applicant: Ultima Genomics, Inc., Newark, CA (US)

(72) Inventors: Florian OBERSTRASS, Menlo Park, CA (US); Theo NIKIFOROV, Carlsbad, CA (US); Mark STAPLETON, Walnut Creek, CA (US); Daniel MAZUR, San Diego, CA (US); Gilad ALMOGY, Palo Alto, CA (US); Ishminder MANN, Fremont, CA (US); Chandan SHEE, Newark, CA (US); Benjamin SCHROEDER, San Mateo, CA (US)

(21) Appl. No.: 18/176,418

(22) Filed: Feb. 28, 2023

Related U.S. Application Data
(63) Continuation of application No. PCT/US21/46951, filed on Aug. 20, 2021.
(60) Provisional application No. 63/184,582, filed on May 5, 2021.
(60) Provisional application No. 63/068,939, filed on Aug. 21, 2020.
(60) Provisional application No. 63/110,277, filed on Nov. 5, 2020.

(51) Int. Cl. C12Q 1/6874; C12Q 1/6855

(52) U.S. Cl. CPC C12Q 1/6874; C12Q 1/6855

(57) ABSTRACT

The present disclosure provides compositions, methods, and systems for enrichment and analysis of template nucleic acid molecules, e.g., of a biological sample. Further provided herein are methods of using a plurality of unique bead species each comprising a unique set of identical primer molecules.

### CROSS REFERENCE

This application is a continuation of International Patent Application No. PCT/US2021/046951, filed on Aug. 20, 2021, which claims the benefit of U.S. Provisional Application Nos. 63/184,582, filed May 5, 2021, 63/068,939, filed Aug. 21, 2020, and 63/110,277, filed Nov. 5, 2020, each of which is incorporated by reference herein in its entirety.

### SEQUENCE LISTING

The instant application contains a Sequence Listing which has been submitted electronically in XML format and is hereby incorporated by reference in its entirety. Said XML copy, created on May 31, 2023, is named 51024-740_301_SL.xml and is 13,191 bytes in size.

### BACKGROUND

Advances in the study of biological molecules have been led, in part, by improvements in technologies used to characterize molecules and/or their biological reactions. In particular, the study of nucleic acids has benefited from developing technologies used for sequence analysis. Sequencing of nucleic acids has various applications in the fields of molecular biology and medicine (e.g., diagnosis and treatment monitoring). Nucleic acid sequencing may provide information that may be used to diagnose a certain condition in a subject and/or tailor a treatment plan. Sequencing is widely used for molecular biology applications, including vector designs, gene therapy, vaccine design, industrial strain design and verification. The way in which an eventual sequence analysis is performed may play a role in the type and quality of information that may be obtained in such analysis.

### SUMMARY

Recognized herein is the need for methods, processes, and compositions for increasing the efficiency, sensitivity, and accuracy of methods for analyzing and/or processing nucleic acid samples. The present disclosure provides methods and compositions for analyzing and/or processing template (or sample) nucleic acid molecules (e.g., those found in biological samples) with high accuracy and sensitivity and efficient reagent usage. Provided herein are methods and compositions that allow for the production and use of unique bead species in template nucleic acid amplification reaction, wherein each unique bead species can comprise a unique set of identical primers. Each primer of each unique bead can be configured to hybridize with one specific template nucleic acid molecule (e.g., one of a biological sample). Thus, unique bead species may allow for efficient amplification and analysis of specific template nucleic acid molecules of a sample, e.g., without the need for partitioning. The present disclosure further provides methods for enriching amplified primers by, e.g., removing unreacted or non-amplified primers from the mixture prior to analysis (e.g., sequencing). Such enrichment can be performed on substrate surface using bioconjugation strategies to selectively bind amplified or “positive” beads to surface and remove any unreacted or non-amplified from the mixture prior to analysis.

In an aspect, provided is a method for processing supports, comprising: (a) providing a plurality of supports, wherein the plurality of supports comprises a plurality of surface primer molecules coupled thereto, wherein a plurality of template nucleic acid molecules are immobilized to a support of the plurality of supports via a first set of surface primer molecules of the plurality of surface primer molecules, wherein a second set of surface primer molecules of the plurality of surface primer molecules is not coupled to the plurality of template nucleic acid molecules; and (b) selectively removing or degrading the second set of surface primer molecules from at least a subset of the plurality of supports to yield a plurality of processed supports, wherein the plurality of processed supports comprises the plurality of template nucleic acid molecules.

In some embodiments, the plurality of template nucleic acid molecule comprises an original nucleic acid molecule from a biological sample or derivative thereof.

In some embodiments, the plurality of template nucleic acid molecule comprises a plurality of amplification products.

In some embodiments, the plurality of template nucleic acid molecules are double-stranded nucleic acid molecules. In some embodiments, (b) comprises contacting the plurality of supports with a reaction mixture comprising a single-strand binding moiety that selectively removes or degrades single-stranded nucleic acid molecules.

In some embodiments, the single-strand binding moiety comprises an enzyme that specifically digests single-stranded nucleic acid molecules. In some embodiments, the enzyme is selected from the group consisting of exonuclease I (ExoI), exonuclease T (ExoT), exonuclease VII (ExoVII), and Mung Bean nuclease (nuclease MB).

In some embodiments, the plurality of template nucleic acid molecules are single-stranded nucleic acid molecules. In some embodiments, (b) comprises contacting the plurality of supports with a reaction mixture comprising a plurality of single-stranded nucleic acid molecules that have at least partial sequence complementarity with the plurality of surface primer molecules, to generate a plurality of double-stranded nucleic acid molecules coupled to the plurality of supports, wherein at least a subset of the plurality of double-stranded nucleic acid molecules comprises at least a subset of the plurality of single-stranded nucleic acid molecules coupled to the second set of surface primer molecules.

In some embodiments, the removing or degrading in (b) comprises contacting the plurality of double stranded nucleic acid molecules with an enzyme. In some embodiments, the enzyme is an exonuclease with 3′ to 5′ activity on double-stranded nucleic acid molecules. In some embodiments, the exonuclease is exonuclease III (ExoIII).

In some embodiments, the plurality of single-stranded nucleic acid molecules have sequence identity.

In some embodiments, an additional subset of the plurality of double-stranded nucleic acid molecules comprises an additional subset of the plurality of single-stranded nucleic acid molecules coupled to at least a subset of the first set of surface primer molecules, wherein the additional subset of the plurality of double-stranded nucleic acid molecules have a 3′ overhang of greater than four bases.

In some embodiments, the at least the subset of the plurality of double-stranded nucleic acid molecules are selected from the group consisting of blunt-ended double-stranded nucleic acid molecules, partially double-stranded nucleic acid molecules with a 5′ overhang, and partially double-stranded nucleic acid molecules with a 3′ overhang of less than four bases.

In some embodiments, a single-stranded nucleic acid molecule of the plurality of single-stranded nucleic molecules comprises a blocking moiety at a 3′ end. In some embodiments, the blocking moiety is a phosphorothioate moiety.

In some embodiments, the plurality of supports is a plurality of beads.

In some embodiments, the plurality of template nucleic acid molecules have sequence identity.

In some embodiments, the plurality of surface primer molecules have sequence identity.

In some embodiments, at least about 50%, 60%, 70%, 75%, 80%, 85%, 90%, 95%, 97%, or 99% of the plurality of surface primer molecules have sequence identity.

In some embodiments, the plurality of supports comprises a first set of supports and a second set of supports, wherein the first set of supports comprises the support, wherein the second set of supports comprises at least a subset of the second set of surface primer molecules.

In some embodiments, the first set of supports comprises another subset of the second set of surface primer molecules.

In some embodiments, each of the first set of supports comprises a template sequence from a plurality of template sequences, wherein the plurality of template sequences is absent from the second set of supports, and further comprising, subsequent (b), isolating the first set of supports from the second set of supports.

In some embodiments, the method further comprises bringing the plurality of processed supports in contact with a surface to immobilize the plurality of processed supports to the surface.

In some embodiments, immobilizing the plurality of processed supports to the surface comprises hybridization of a template nucleic acid molecule of the plurality of template nucleic acid molecules to a primer molecule coupled to the surface.

In some embodiments, immobilizing the plurality of processed supports to the surface comprises using biotin coupled to a template nucleic acid molecule of the plurality of template nucleic acid molecules and streptavidin coupled to the surface.

In some embodiments, immobilizing the plurality of processed supports to the surface comprises using a modified azide group of a template nucleic acid molecule of the plurality of template nucleic acid molecules and a cyclooctyne coupled to the surface.

In some embodiments, immobilizing the plurality of processed supports to the surface comprises using a modified thiol group of a template nucleic acid molecule of the plurality of template nucleic acid molecules and a maleimide coupled to the surface.

In some embodiments, the method further comprises washing from the surface a subset of processed supports, wherein the subset of processed supports is not coupled to a template sequence of a plurality of template sequences.

In some embodiments, the method further comprises sequencing the plurality of template nucleic acid molecules while coupled to the support.

In some embodiments, the method further comprises, prior to (a), subjecting a plurality of initial supports comprising the plurality of surface primer molecules and a sample molecule, or derivate thereof, to polymerase chain reaction (PCR), to provide the plurality of supports in (a), wherein the plurality of template nucleic acid molecules have sequence identity or sequence complementarity to a sequence of the sample molecule.

In some embodiments, the plurality of template nucleic acid molecules are covalently linked to the first set of surface primer molecules, via an enzymatic ligation or extension reaction.

In another aspect, provided is a method comprising: (a) providing a solution comprising: (i) a first support and a second support, wherein the first support comprises a first primer of a plurality of first primers and the second support comprises a second primer of a plurality of second primers, wherein the first primer and the second primer comprise different nucleic acid sequences; (ii) a first nucleic acid molecule and a second nucleic acid molecule, wherein the first nucleic acid molecule comprises a first adapter sequence configured to hybridize to the first primer, wherein the second nucleic acid molecule comprises a second adapter sequence configured to hybridize to the second primer; and (iii) one or more reagents; (b) (i) using the first nucleic acid molecule and the one or more reagents to generate a plurality of first amplification products, wherein at least a subset of the plurality of first amplification products are attached to the first support via the plurality of first primers; and (ii) using the second nucleic acid molecule and the one or more reagents to generate a plurality of second amplification products, wherein at least a subset of the plurality of second amplification products are attached to the second support via the plurality of second primers; and (c) assaying (i) first amplification products of the plurality of first amplification products attached to the first support, or derivatives thereof, and (ii) second amplification products of the plurality of second amplification products attached to the second support, or derivatives thereof, to identify a first sequence of the first nucleic acid molecule and a second sequence of the second nucleic acid molecule.

In some embodiments, (b) is performed within a bulk solution.

In some embodiments, prior to (b), the first support, the second support, the first nucleic acid molecule, and the second nucleic acid molecule are included within a bulk solution.

In some embodiments, the first nucleic acid molecule is immobilized to the first support via the first primer and the second nucleic acid molecule is immobilized to the second support via the second primer, wherein the method further comprises, prior to (b), isolating the first support and the second support from supports that do not have any nucleic acid molecule immobilized thereon.

In some embodiments, the first primer comprises a first extended primer region that is absent from other first primers in the plurality of first primers and the second primer comprises a second extended primer region that is absent from other second primers in the plurality of second primers, and wherein the first nucleic acid molecule is immobilized to the first support via the first extended primer region and the second nucleic acid molecule is immobilized to the second support via the second extended primer region.

In some embodiments, each first primer of the plurality of first primers are identical.

In some embodiments, the method further comprises, prior to (b), generating a first partition comprising the first support and the first nucleic acid molecule and a second partition comprising the second support and the second nucleic acid molecule.

In some embodiments, the first partition and the second partition are droplets of a plurality of droplets.

In some embodiments, the first partition and the second partition comprise the one or more reagents.

In some embodiments, the plurality of first amplification products are generated within the first partition, and the plurality of second amplification products are generated within the second partition.

In some embodiments, the method further comprises recovering the plurality of first amplification products from the first partition and the plurality of second amplification products from the second partition.

In some embodiments, the first support and the second support are a first particle and a second particle.

In some embodiments, the first particle is a first bead, and the second particle is a second bead.

In some embodiments, the first support and the second support are immobilized to a substrate surface.

In some embodiments, the first support and the second support are immobilized to the substrate surface prior to (b).

In some embodiments, the first support and the second support are immobilized to the substrate surface subsequent to (b).

In some embodiments, the substrate comprises a substantially planar array.

In some embodiments, the substrate comprises a plurality of individually addressable locations.

In some embodiments, the first support and the second support are immobilized to the substrate surface via one or more oligonucleotide molecules.

In some embodiments, the first nucleic acid molecule comprises a third adapter sequence disposed at an end distal to the first adapter sequence and the second nucleic acid molecule comprises a fourth adapter sequence disposed at an end distal to the second adapter sequence.

In some embodiments, the third adapter sequence and the fourth adapter sequence comprise identical nucleic acid sequences.

In some embodiments, the one or more reagents comprise a plurality of additional primers, wherein an additional primer of the plurality of additional primers is configured to hybridize to the third adapter sequence and the fourth adapter sequence.

In some embodiments, the third adapter sequence and the fourth adapter sequence comprise different nucleic acid sequences.

In some embodiments, the one or more reagents comprise a plurality of additional primers, wherein the plurality of additional primers comprises a first additional primer configured to hybridize to the third adapter sequence of the first nucleic acid molecule and a second additional primer configured to hybridize to the fourth adapter sequence of the second nucleic acid molecule.

In some embodiments, (b) comprises performing polymerase chain reaction (PCR).

In some embodiments, (b) comprises performing recombinase polymerase amplification (RPA).

In some embodiments, the one or more reagents comprises a recombinase enzyme.

In some embodiments, the one or more reagents comprises a binding protein.

In some embodiments, the one or more reagents comprises a crowding agent.

In some embodiments, the first nucleic acid molecule is single-stranded.

In some embodiments, the first nucleic acid molecule is double-stranded.

In some embodiments, the method further comprises ligating the first nucleic acid molecule to the first support.

In some embodiments, the first adapter sequence comprises one or more cleavable moieties. In some embodiments, the one or more cleavable moieties are selected from the group consisting of uracils and ribonucleotide bases.

In some embodiments, the first nucleic acid molecule comprises a third adapter sequence comprising an affinity tag. In some embodiments, the affinity tag comprises a biotin. In some embodiments, the third adapter sequence further comprises one or more cleavable moieties.

In another aspect, provided is a composition, comprising: a mixture comprising a plurality of supports, wherein the plurality of supports comprises: (i) a first support comprising, immobilized thereto, a first primer of a plurality of first primers configured to hybridize to a first exogenous adapter sequence, and (ii) a second support comprising, immobilized thereto, a second primer of a plurality of second primers configured to hybridize to a second exogenous adapter sequence, wherein the first exogenous adapter sequence and the second exogenous adapter sequence comprise different sequences, and wherein the first primer and the second primer comprise different sequences.

In some embodiments, the plurality of supports is a plurality of particles.

In some embodiments, the plurality of particles is a plurality of beads.

In some embodiments, the plurality of supports is immobilized to a substrate surface.

In some embodiments, the plurality of supports is immobilized to the substrate surface via one or more oligonucleotide molecules.

In some embodiments, the plurality of supports is provided in a solution.

In some embodiments, the solution comprises one or more reagents.

In some embodiments, the one or more reagents comprise one or more additional primers, enzymes, proteins, crowding agents, buffers, cations, or a combination thereof.

In some embodiments, the mixture comprises a plurality of partitions.

In some embodiments, the plurality of partitions is a plurality of droplets.

In some embodiments, the first primer comprises a first extended primer region that is absent from other first primers in the plurality of first primers and the second primer comprises a second extended primer region that is absent from other second primers in the plurality of second primers, and wherein the first nucleic acid molecule is immobilized to the first support via the first extended primer region and the second nucleic acid molecule is immobilized to the second support via the second extended primer region.

In some embodiments, each first primer of the plurality of first primers are identical.

In another aspect, provided is a method for processing supports, comprising: (a) providing a mixture of supports comprising a first support, wherein the first support comprises immobilized thereto a surface primer sequence hybridized to an adapter sequence of a template nucleic acid molecule of a plurality of template nucleic acid molecules; (b) contacting the mixture with a modified nucleotide comprising a first reactive moiety to incorporate the modified nucleotide to extend the surface primer sequence according to the adapter sequence of the template nucleic acid molecule, wherein the first reactive moiety is configured for capture by a second reactive moiety; (c) isolating the first support from the mixture of supports onto a substrate surface by capturing the first reactive moiety using the second reactive moiety immobilized to the substrate surface; and (d) contacting the first support with an amplification reaction mixture on the surface.

In some embodiments, subsequent to (c), the substrate surface comprises a plurality of supports immobilized thereto, including the first support, each support of the plurality of supports associated with a different template sequence of the plurality of template nucleic acid molecules, and wherein (d) comprises contacting the plurality of supports with the amplification reaction mixture.

In some embodiments, the plurality of supports comprises a plurality of surface primer populations, wherein each surface primer population comprises a primer sequence unique to the surface primer population in the plurality of surface primer populations, and wherein the plurality of template nucleic acid molecules comprises different adapter sequences configured to hybridize with different primer sequences of the plurality of supports.

In some embodiments, the plurality of supports comprises at least 2, at least 5, at least 5, at least 10, at least 15, at least 20, at least 30, at least 40, at least 50, or at least 100 different primer sequences.

In some embodiments, the method further comprises amplifying the template nucleic acid molecule attached to the first support go generate a plurality of amplification products attached to the first support.

In some embodiments, the amplifying comprises performing polymerase chain reaction (PCR).

In some embodiments, the amplifying comprises performing recombinase polymerase amplification.

In some embodiments, the amplification reaction mixture comprises a reagent for increasing viscosity.

In some embodiments, the reagent is a crowding agent.

In some embodiments, the crowding agent is PEG.

In some embodiments, the substrate surface comprises a plurality of physical features configured to impede travel of amplification product molecules from a first reaction space comprising a first immobilized support to a second reaction space comprising a second immobilized support.

In some embodiments, the plurality of physical features comprises a plurality of recesses.

In some embodiments, the first nucleic acid molecule is single-stranded.

In some embodiments, the first nucleic acid molecule is double-stranded.

In some embodiments, the method further comprises ligating the first nucleic acid molecule to the first support.

In some embodiments, the first adapter sequence comprises one or more cleavable moieties.

In some embodiments, the one or more cleavable moieties are selected from the group consisting of uracils, ribonucleotide bases, methylated nucleotide bases, spacers, and abasic sites.

In some embodiments, the first nucleic acid molecule comprises a third adapter sequence comprising an affinity tag.

In some embodiments, the affinity tag comprises a biotin.

In some embodiments, the third adapter sequence further comprises one or more cleavable moieties.

In another aspect, provided herein is a method for pre-enriching a mixture of supports, comprising: (a) providing (i) the mixture of supports comprising a first support, wherein the first support comprises immobilized thereto a surface primer sequence and (ii) a plurality of template nucleic acid molecules, wherein a template nucleic acid molecule of the plurality of template nucleic acid molecules comprises a cleavable moiety and a first reactive moiety that is configured for capture by a second reactive moiety; (b) attaching the template nucleic acid molecule to the first support via the surface primer sequence; (c) isolating the first support from the mixture of supports onto a substrate surface comprising the second reactive moiety immobilized thereto, wherein the isolating is performed by capturing the first reactive moiety using the second reactive moiety; and (d) cleaving the cleavable moiety, thereby generating a pre-enriched support.

In some embodiments, subsequent to (b), the mixture of supports comprises a plurality of unbound supports that do not comprise any template nucleic acid molecules of the plurality of template nucleic acid molecules attached thereto.

In some embodiments, (c) comprises separating the first support from the plurality of unbound supports.

In some embodiments, the method further comprises collecting the plurality of unbound supports and repeating (a) using the plurality of unbound supports and an additional plurality of template nucleic acid molecules.

In some embodiments, the additional plurality of template nucleic acid molecules comprise at least a portion of the plurality of template nucleic acid molecules.

In some embodiments, the additional plurality of template nucleic acid molecules comprise different template nucleic acid molecules than the plurality of template nucleic acid molecules.

In some embodiments, prior to the repeating, the plurality of unbound supports are contacted with a blocking moiety.

In some embodiments, the blocking moiety comprises biotin or biocytin.

In some embodiments, the blocking moiety comprises biocytin, and wherein the biocytin is provided at a concentration of at least 100 picomolar.

In some embodiments, the substrate surface comprising the second reactive moiety immobilized thereto is a bead.

In some embodiments, the bead is a magnetic bead.

In some embodiments, (c) comprises using a magnetic field to isolate the first support from the mixture of supports.

In some embodiments, a ratio of a concentration of the mixture of supports and a concentration of the plurality of template nucleic acid molecules is greater than 1. In some embodiments, the ratio is about 10.

In some embodiments, the cleavable moiety comprises a uracil, a ribonucleotide, a spacer, or a methylated nucleotide. In some embodiments, the spacer is a dSpacer or a C3 spacer. In some embodiments, (d) comprises using APE1 enzyme to cleave the spacer. In some embodiments, the cleavable moiety is a methylated nucleotide and (d) comprises using MspJI to cleave the methylated nucleotide. In some embodiments, the cleavable moiety is a uracil and wherein (d) comprises using a uracil D glycosylase (UDG) to cleave the uracil. In some embodiments, the cleavable moiety is a ribonucleotide and wherein (d) comprises using a RNase to cleave the ribonucleotide.

In some embodiments, the first reactive moiety comprises a biotin moiety and the second reactive moiety comprises a streptavidin moiety. In some embodiments, first reactive moiety comprises an overhang oligonucleotide sequence and the second reactive moiety comprises a capture oligonucleotide complementary to at least a portion of the overhang oligonucleotide sequence.

In some embodiments, the template nucleic acid molecule comprises an adapter sequence and (b) comprises hybridizing the adapter sequence to the surface primer sequence and performing a ligation reaction. In some embodiments, the ligation reaction is performed using a ligase. In some embodiments, the ligation reaction is performed using a ligase and a polymerase. In some embodiments, the polymerase is a Taq polymerase. In some embodiments, the adapter sequence comprises an additional cleavable moiety.

In some embodiments, the template nucleic acid molecule is double stranded. In some embodiments, the method further comprises, prior to (a), generating the double-stranded template nucleic acid molecule. In some embodiments, the generating is performed by ligating a pair of adapters to a double stranded insert nucleic acid molecule (also referred to herein as a “template molecule” or “template nucleic acid molecule” interchangeably), wherein an adapter of the pair of adapters comprises the cleavable moiety and the first reactive moiety. In some embodiments, the generating is performed by performing an amplification reaction using a pair of adapters and an insert nucleic acid molecule, wherein an adapter of the pair of adapters comprises the cleavable moiety and the first reactive moiety.

In some embodiments, the generating is performed by ligating a pair of adapters to a single stranded insert nucleic acid molecule, wherein an adapter of the pair of adapters comprises the cleavable moiety and the first reactive moiety.

In some embodiments, the adapter comprises an overhang sequence that is complementary to a portion of the single stranded insert nucleic acid molecule. In some embodiments, the overhang sequence comprises a random N-mer. In some embodiments, the overhang sequence is located at a 5′ terminus of the adapter. In some embodiments, the overhang sequence comprises a blocking moiety at the 5′ terminus.

In some embodiments, another adapter of the pair of adapters comprises an overhang sequence that is complementary to a portion of the single stranded insert nucleic acid molecule. In some embodiments, the overhang sequence comprises a random N-mer. In some embodiments, the overhang sequence is located at a 3′ terminus of the adapter. In some embodiments, the overhang sequence comprises a blocking moiety at the 3′ terminus. In some embodiments, the single stranded insert nucleic acid molecule is generated using single stranded DNA binding proteins. In some embodiments, the generating comprises, prior to the ligating, annealing the pair of adapters to the single stranded insert nucleic acid molecule to generate an adapter-insert complex, and phosphorylating the adapter-insert complex. In some embodiments, the phosphorylating is performed using a T4 polynucleotide kinase.

In some embodiments, the adapter is ligated to a 3′ end of the single stranded insert nucleic acid molecule, and subsequently, an additional adapter of the pair of adapters is ligated to a 5′ end of the single stranded insert nucleic acid molecule.

In some embodiments, prior to the ligating, the single stranded insert nucleic acid molecule is treated with bisulfate.

In some embodiments, (b) is mediated using a splint molecule. In some embodiments, the splint molecule comprises a first sequence complementary to a sequence of the template nucleic acid molecule and a second sequence complementary to the surface primer sequence. In some embodiments, the splint molecule is provided at a concentration that is lower than a concentration of the plurality of template nucleic acid molecules. In some embodiments, the splint molecule is provided at a concentration that is at least one order of magnitude lower than a concentration of the plurality of template nucleic acid molecules. In some embodiments, the method further comprises selecting the concentration. In some embodiments, (b) is further mediated using a bridge molecule. In some embodiments, the bridge molecule comprises a first sequence complementary to a sequence of the template nucleic acid molecule and a second sequence complementary to a first splint sequence of the splint molecule, and wherein the splint molecule comprises a second splint sequence complementary to the surface primer sequence.

In another aspect, disclosed herein is a composition, comprising: a single-stranded insert nucleic acid molecule; a first adapter comprising (i) a cleavable moiety (ii) a first reactive moiety that is configured for capture by a second reactive moiety and (iii) an overhang sequence that is complementary to a portion of the single-stranded insert nucleic acid molecule; and a second adapter comprising an additional sequence that is complementary to another portion of the single-stranded insert nucleic acid molecule.

In some embodiments, the overhang sequence or the additional sequence comprises a random N-mer.

In some embodiments, the overhang sequence is located at a 5′ terminus of the first adapter. In some embodiments, the 5′ terminus comprises a blocking moiety.

In some embodiments, the additional sequence is located at a 3′ terminus of the second adapter. In some embodiments, the 3′ terminus comprises a blocking moiety.

In some embodiments, the cleavable moiety comprises a uracil, a ribonucleotide, a methylated nucleotide, a hairpin sequence, or a spacer.

In some embodiments, the first reactive moiety comprises a biotin moiety and the second reactive moiety comprises a streptavidin moiety.

In some embodiments, the first reactive moiety comprises an overhang oligonucleotide sequence and the second reactive moiety comprises a capture oligonucleotide complementary to at least a portion of the overhang oligonucleotide sequence.

In some embodiments, the second adapter comprises a binding sequence configured to be captured by a capture sequence of a support. In some embodiments, the support is a bead. In some embodiments, the composition further comprises the support. In some embodiments, the composition further comprises the second reactive moiety.

In some embodiments, the composition further comprises one or more proteins selected from the group consisting of a single stranded binding protein, a ligase, and a kinase.

In some embodiments, the first adapter or the second adapter comprises a barcode sequence.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a method of the present disclosure for surface enrichment of template nucleic acid molecules (e.g., from a library of template nucleic acid molecules), in accordance with some embodiments of the present disclosure.

FIG. 2 illustrates a method of the present disclosure for producing single bead species and using such single bead species for surface enrichment of template nucleic acid molecules (e.g., from a library of template nucleic acid molecules).

FIG. 3A illustrates a method for using multiple bead species to capture different template nucleic acid molecules of a sample.

FIG. 3B illustrates template nucleic acid molecules that have been modified such that each template molecule comprises a unique first adapter sequence “B” or “C” (e.g., for hybridization with a surface primer attached to a bead) and a unique second adapter sequence “A” or “D” (e.g., for hybridization with a solution primer).

FIGS. 3C-3G illustrate a method for on-support bridge amplification.

FIG. 4 illustrates another method for amplification.

FIG. 5A, FIG. 5B, FIG. 5C, and FIG. 5D illustrate the methods of the present disclosure performed on an open surface.

FIG. 6A and FIG. 6B illustrate an embodiment of the present disclosure where the second primer is attached to the surface.

FIG. 6C, FIG. 6D and FIG. 6E illustrate an embodiment of the present disclosure where each of the colony locations on a surface have a different first primer.

FIG. 6F illustrates an embodiment of the present disclosure where each surface cluster comprises unique primer species.

FIG. 7A, FIG. 7B, FIG. 7C, and FIG. 7D illustrate an embodiment of the present disclosure that includes a second slow extension step.

FIG. 8 illustrates an example where an adapter is attached to each end of the template nucleic acid molecule.

FIG. 9 illustrates an example of generating an extended support.

FIGS. 10A-10B illustrate examples of separating an extended support from a solution by applying a magnetic force.

FIG. 11 illustrates another example of separating an extended support from a solution by applying a magnetic force.

FIGS. 12A-12B illustrates an example pre-enrichment method for generating a plurality of pre-enriched supports.

FIG. 13 shows the results of amplification using pre-enrichment procedures.

FIG. 14 shows the presence of enriched beads captured, at different extension primer input concentrations.

FIG. 15 shows the presence of extension primer sequences in enriched beads, at different extension primer input concentrations.

FIG. 16 shows the presence of amplified beads, at different extension primer input concentrations.

FIG. 17 shows polyclonality in amplified beads, at different extension primer input concentrations.

FIG. 18A illustrates positive and negative beads that can comprise unreacted, e.g., non-amplified, surface primer attached to their surface.

FIG. 18B illustrates positive and negative beads in which unreacted, e.g., non-amplified, surface primer are hybridized using a second set of nucleic acid molecules.

FIG. 18C illustrates positive and negative beads in which unreacted, e.g., non-amplified, surface primers have been removed using, e.g., an exonuclease, in order to increase sensitivity in sequencing experiments of this disclosure.

FIG. 18D illustrates an example workflow for positive and negative beads in which unreacted, e.g., non-amplified, surface primers are removed using, e.g., single strand binding moieties.

FIG. 18E illustrates another example workflow for positive and negative beads in which unreacted, e.g., non-amplified, surface primers are removed using, e.g., single strand binding moieties.

FIG. 19A and FIG. 19B illustrate methods for loading beads onto a substrate. FIG. 19A illustrates a method for loading beads onto specific regions of a substrate. FIG. 19B illustrates a method for loading a subset of beads onto specific regions of a substrate.

FIG. 20A illustrates an example method for enriching positive supports (e.g., beads) on a substrate surface by incorporating nucleotides modified with reactive moieties into the support-template complex, e.g., during primer extension.

FIG. 20B illustrates an example method for enriching positive supports (e.g., beads) on a substrate surface by incorporating nucleotides modified with reactive moieties into template sequences (e.g., at the 3′ or 5′ end).

FIG. 21 illustrates an example workflow for sample preparation and analysis, according to systems, compositions, and methods of the present disclosure.

FIGS. 22A-22F illustrate example workflows for pre-enrichment of template nucleic acid molecules, in accordance with some embodiments of the present disclosure.

FIG. 23 illustrates an example workflow for recycling of supports, as described herein.

FIG. 24 schematically illustrates an example workflow for recycling of supports, as described herein.

FIG. 25 illustrates example data of a cleavable moiety.

FIG. 26 illustrates example data of pre-enrichment efficiencies using a polymerase for attachment of template nucleic acid molecules to a support.

FIGS. 27A-27C illustrate example flow cytometry data demonstrating pre-enrichment efficiencies using a polymerase for attachment of template nucleic acid molecules to a support.

FIG. 28 illustrates additional example data of pre-enrichment efficiencies using a polymerase for attachment of template nucleic acid molecules to a support.

FIG. 29 illustrates additional example flow cytometry data demonstrating pre-enrichment efficiencies using a polymerase for attachment of template nucleic acid molecules to a support.

FIG. 30 illustrates an example splint molecule and bridge molecule for attachment of template nucleic acid molecules to a support. Figure discloses SEQ ID NOS 2-5, respectively, in order of appearance.

FIG. 31 shows example data of pre-enrichment efficiencies using a splint molecule and bridge molecule for attachment of template nucleic acid molecules to a support.

FIGS. 32A-32C shows example oligonucleotides or modified bases, as described herein. FIG. 32A shows an example hairpin oligonucleotide. FIG. 32A discloses SEQ ID NO: 6. FIG. 32B shows a dSpacer moiety and FIG. 32C shows a C3 Spacer (linker).

FIG. 33 shows an example workflow for generating a template nucleic acid molecule (e.g., of a library of template nucleic acid molecules) by ligating adapter molecules to template sequences.

FIGS. 34A-34C illustrate example adapter molecules which may be ligated to template sequences to generate template nucleic acid molecules (e.g., of a library of template nucleic acid molecules).

FIG. 35 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Ranges may be expressed herein as from “about” one particular value, and/or to “about” another particular value. The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “amplification,” as used herein, generally refers to the production of one or more copies of a nucleic acid molecule or an extension product (e.g., a product of a primer extension reaction on the nucleic acid molecule). Amplification of a nucleic acid molecule may yield a single strand hybridized to the nucleic acid molecule, or multiple copies of the nucleic acid molecule or complement thereof. An amplicon may be a single-stranded or double-stranded nucleic acid molecule that is generated by an amplification procedure from a starting template nucleic acid molecule. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. An amplification reaction may be, for example, a polymerase chain reaction (PCR), such as an emulsion polymerase chain reaction (ePCR; e.g., PCR carried out within a microreactor such as a well or droplet).

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA.

The term “clonal,” as used herein, generally refers to a population of nucleic acids for which a substantial portion (e.g., greater than 50%, 60%, 70%, 80%, 90%, 95%, or 99%) of its members have substantially identical sequences. Members of a clonal population of nucleic acid molecules may have sequence homology to one another. In some instances, such members may have sequence homology to a template nucleic acid molecule. In some instances, such members may have sequence homology to a complement of the template nucleic acid molecule (if single stranded). The members of the clonal population may be double stranded or single stranded. Members of a population may not be 100% identical or complementary because, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

The term “complementary sequence,” as used herein, generally refers to a sequence that hybridizes to another sequence or has sequence complementarity with such other sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “polymerizing enzyme,” as used herein, generally refers to a substance catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. A polymerizing enzyme may be a polymerase such as a nucleic acid polymerase. A polymerase may be naturally occurring or synthesized. A polymerase may have relatively high processivity, namely the capability of the polymerase to consecutively incorporate nucleotides into a nucleic acid template without releasing the nucleic acid template. A polymerizing enzyme may be a transcriptase. Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase, 029 (phi29) DNA polymerase, Taq polymerase, Tth polymerase, Tli polymerase, Pfu polymerase, Pwo polymerase, VENT polymerase, DEEPVENT polymerase, EXTaq polymerase, LA-Taq polymerase, Sso polymerase, Poc polymerase, Pab polymerase, Mth polymerase, ES4 polymerase, Tru polymerase, Tac polymerase, Tne polymerase, Tma polymerase, Tea polymerase, Tih polymerase, Tfi polymerase, Platinum Taq polymerases, Tbr polymerase, Tfl polymerase, Pfutubo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. A polymerase may be a single subunit polymerase.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novère N, MELTING, a flexible platform to predict the melting temperatures of nucleic acids. BMC Bioinformatics. 2012 May 16; 13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine—(A), thymine—(T), cytosine—(C), uracil—(U), or guanine—(G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). As disclosed herein, the terms cleavable and excisable are used interchangeably. In some cases, the label may be luminescent; that is, fluorescent or phosphorescent. Labels may be quencher molecules. The term “quencher,” as used herein refers to a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. In some cases, as described elsewhere herein, labelling with a quencher can occur after nucleotide or nucleotide analog incorporation. Dyes and labels may be incorporated into nucleic acid sequences. Dyes and labels may also be incorporated into linkers, such as linkers for linking one or more beads to one another. Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; CySQ and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other.

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle or support. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. A coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction. In another example, a particle may be coupled to a planar support via a magnetic interaction. In another example, a particle may be coupled to a planar support via a covalent interaction. Similarly, a nucleic acid molecule may be coupled to a particle via a covalent interaction. Alternatively or additionally, a nucleic acid molecule may be coupled to a particle via a non-covalent interaction. A coupling between a first object and a second object may comprise a labile moiety, such as an moiety comprising an ester, vicinal diol, phosphodiester, peptidic, glycosidic, sulfone, Diels-Alder, or similar linkage. The strength of a coupling between a first object and a second object may be indicated by a dissociation constant, Kd, that indicates the inclination of a coupled object comprising a first object and a second object to dissociate into the uncoupled first and second objects and may be expressed as a ratio of dissociated (e.g., uncoupled) objects to coupled objects. A smaller dissociation constant is generally indicative of a stronger coupling between coupled objects.

Coupled objects and their corresponding uncoupled components may exist in dynamic equilibrium with one another. For example, a solution comprising a plurality of coupled objects each comprising a first object and a second object may also include a plurality of first objects and a plurality of second objects. At a given point in time, a given first object and a given second object may be coupled to one another or the objects may be uncoupled; the relative concentrations of coupled and uncoupled components throughout the solution will depend upon the strength of the coupling between the first and second objects (reflected in the dissociation constant). For example, a binding moiety may be coupled to a nucleic acid molecule to provide a binding complex. In a solution comprising a plurality of binding complexes each comprising a binding moiety coupled to a nucleic acid molecule, the plurality of binding complexes may exist in equilibrium with their constituent nucleic acid molecules and binding moieties. The association between a given nucleic acid molecule and a given binding moiety may be such that, at a given point in time, at least 50%, such as at least 60%, 65%, 70%, 75%, 80%, 85%, 90%, 95%, 98%, or more, of the nucleic acid molecules may be components of a binding complex of the plurality of binding complexes.

The term “detector,” as used herein, generally refers to a device that is capable of detecting a signal, such as a signal indicative of the presence or absence of an incorporated nucleotide or nucleotide analog. A detector may include optical and/or electronic components that may detect signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

The term “sequencing,” as used herein, generally refers to a process for generating or identifying a sequence of a biological molecule, such as a nucleic acid molecule. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively or in addition, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index or other physical characteristic. A subject may have a given ethnic or racial heritage, place of birth or residence, nationality, disease or remission state, family medical history, or other characteristic.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively or in addition, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively or in addition, cell-free nucleic acid molecules may be released into bodily fluids from healthy cells.

A biological sample obtained directly from a subject may not have been further processed following being obtained from the subject. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively or in addition, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid”, “target nucleic acid”, “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpes virus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

The methods described herein may be conducted in a reaction vessel (e.g., a droplet in an emulsion, or a well among a plurality of wells). Any suitable reaction vessel may be used. A reaction vessel may comprise a body that includes an interior surface, an exterior surface, and, in some cases, an open end and an opposing closed end. In some cases, a reaction vessel may not comprise an open or closed end. For example, a reaction vessel may be a droplet. In other cases, a reaction vessel may comprise a cap, which cap may be configured to contact the body at an open end, such that when contact is made the open end of the reaction vessel is closed. The cap may be permanently associated with the reaction vessel such that it remains attached to the reaction vessel in open and closed configurations. The cap may be removable, such that when the reaction vessel is open, the cap is separated from the reaction vessel. A reaction vessel such as a flow cell chamber (e.g., a flow cell chamber comprising a water-in-oil emulsion or a plurality of wells) may comprise one or more inlets or outlets, which inlets or outlets may be used to provide and remove reagents for use in a reaction. Reagents may be moved in and out of the chamber via pressure and vacuum controls. A reaction vessel as used herein may be sealed, optionally hermetically sealed (e.g., a sealed microwell plate).

A reaction vessel may be of varied size, shape, weight, and configuration. Some reaction vessels may be substantially round or oval tubular shaped. Some reaction vessels may be rectangular, square, diamond, circular, elliptical, or triangular shaped. A reaction vessel may be regularly shaped or irregularly shaped. For example, a reaction vessel that is a droplet (e.g., a droplet in an emulsion, such as an aqueous droplet) may be substantially spherical. A closed end of a reaction vessel (e.g., a well of a microwell plate or flow cell) may have a tapered, rounded, or flat surface. Non-limiting examples of types of a reaction vessel include a tube, a well, a capillary tube, a cartridge, a cuvette, a centrifuge tube, a droplet, or a pipette tip. Reaction vessels may be comprised of any suitable material with non-limiting examples of such materials that include glasses, metals, plastics, immiscible fluids, and combinations thereof. In an example, a reaction vessel may be a droplet, such as an aqueous droplet in an immiscible fluid such as an oil. A reaction vessel may be of any suitable size. For example, a reaction vessel may be an approximately spherical droplet having a diameter of at least about 1 nanometer (nm), 10 nm, 50 nm, 100 nm, 1 micron (μm), 10 μm, 50 μm, 100 μm, 1 millimeter (mm), 10 mm, 50 mm, 100 mm, or 1 centimeter (cm). Alternatively, a reaction vessel may be a well having a diameter of at least about 100 μm, 1 mm, 5 mm, or 10 mm. The depth of a well may be the same as or different than the diameter of the well. For example, the well may have a diameter of about 5 mm and a depth of about 10 mm.

A reaction vessel may be part of a collection or an array of reaction vessels. A collection or an array of reaction vessels may be particularly useful for automating methods and/or simultaneously processing multiple samples. A reaction vessel may be a well of a microwell plate comprised of a number of wells. A reaction vessel may be held in a well of a thermal block of a thermocycler, wherein the block of the thermal cycle comprises multiple wells each capable of receiving a sample vessel. A collection or an array comprised of reaction vessels (e.g., droplets or microwells) may comprise any appropriate number of reaction vessels. A collection or an array of reaction vessels may include at least 1, 2, 3, 4, 5, 6, 7, 8, 9, 10, 20, 30, 40, 50, 100, 200, 300, 400, 500, 1,000, 10,000 or more vessels. For example, a collection or an array of reaction vessels may comprise at least 2, 4, 6, 8, 10, 12, 14, 16, 18, 20, 25, 35, 48, 96, 144, 384, or more reaction vessels. A reaction vessel part of a collection or an array of reaction vessels (e.g., microwells) may also be individually addressable by a fluid handling device, such that the fluid handling device may correctly identify a reaction vessel and dispense appropriate fluid materials into the reaction vessel. Fluid handling devices may be useful in automating the addition of fluid materials to reaction vessels.

In some cases, one or more reaction vessels may be included within another reaction vessel. For example, a plurality of droplets may be included in a container such as a beaker, test tube, flow cell chamber, or other container, or a plurality of wells (e.g., of a microwell plate or flow cell) may be included in a container, such as a flow cell chamber. In an example, a plurality of wells may be provided on a surface of a flow cell chamber, such that a nucleic acid reaction may take place directly on a flow cell. In another example, one or more droplets may be physically constrained to a given area, such as a surface of a container. Droplets may be physically constrained via, for example, an electromagnetic force, such as via a magnetic attraction between a material (e.g., surface) of the container and a material included within the droplet (e.g., a paramagnetic bead or a magnetic label coupled to a bead) or via the use of optical tweezers. In an example, droplets may be constrained within wells (e.g., of a microwell plate or flow cell).

A reaction vessel (e.g., droplet or well) as used herein may comprise multiple thermal zones. Thermal zones may be created within a reaction vessel with the aid of thermal sensitive layering materials within the reaction vessels. In such cases, heating of the thermal sensitive layering materials may be used to release reaction mixtures from one thermal zone to the next. A reaction vessel may comprise 2, 3, 4, 5, 6, 7, 8, 9, 10, 11, 12, 13, 14, 15, 20 or more thermal zones. Thermal zones within a reaction vessel may be achieved by exposing different regions of the reaction vessel to different temperature cycling conditions. For example, different regions of a flow cell chamber (e.g., comprising a plurality of wells and/or droplets) may be subjected to different temperature cycling conditions. Alternatively, one or more reaction vessels of an array or a collection of reaction vessels may be subjected to one or more different thermal zones. For example, a first set of reaction vessels may be placed within a first thermal zone and a second set of reaction vessels may be placed within a second thermal zone (e.g., by physically separating the various reaction vessels). Alternatively or in addition, one or more reaction vessels of an array or a collection of reaction vessels may be subjected to multiple different temperatures (e.g., at different times throughout a process). Temperatures applied to a reaction vessel may be suitable for, for example, initialization of a nucleic acid reaction, annealing of nucleic acid molecules, extension of an annealed nucleic acid molecule (e.g., primer extension), partial or complete denaturation of a double-stranded nucleic acid sequence or portion thereof, or any other useful process. For example, temperatures may be controlled according to a thermocycling protocol. In an example, all or a portion of a reaction vessel may be subjected to a first temperature at a first time for a first duration, and the reaction vessel or portion thereof may subsequently be subjected to a second temperature at a second time for a second duration. The first temperature may be, for example, a temperature suitable for initialization of a nucleic acid reaction (e.g., PCR) or annealing (e.g., hybridization) of a first nucleic acid molecule to a second nucleic acid molecule. The second temperature may be, for example, a temperature suitable for extension of an annealed nucleic acid molecule (e.g., a primer molecule) and/or denaturation of annealed nucleic acid molecules. Additional different temperatures may also be applied. Temperatures may be repeated any suitable number of times (e.g., for any number of thermocycles).

The term “support” or “substrate,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. An amplification substrate can be placed within or on another substrate, for example, where beads used as amplification substrates are disposed (e.g., immobilized) on a planar surface, or where beads used as amplification substrates are disposed (e.g., immobilized) inside of wells.

The term “bead,” as described herein, generally refers to a solid support, resin, gel (e.g., hydrogel), colloid, or particle of any shape and dimensions. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. For example, a bead may comprise one or more polymers bearing one or more magnetic labels. A magnetic bead may be manipulated (e.g., moved between locations or physically constrained to a given location, e.g., of a reaction vessel such as a flow cell chamber) using electromagnetic forces. A bead may have one or more different dimensions including a diameter. A dimension of the bead (e.g., the diameter of the bead) may be less than about 1 mm, less than about 0.1 mm, less than about 0.01 mm, less than about 0.005 mm, from about 1 nm to about 100 nm, from about 1 μm to about 100 μm, or from about 1 mm to about 100 mm.

A collection of beads may comprise one or more beads having the same or different characteristics. For example, a first bead of a collection of beads may have a first diameter and a second bead of the collection of beads may have a second diameter. The first diameter may be the same or approximately the same as or different from the second diameter. Similarly, the first bead may have the same or a different shape and composition than a second bead. In an example, the first bead may comprise a first polymeric material and the second bead may comprise a second polymeric material. The first polymeric material may be the same or different as the second polymeric material. The first bead may comprise a first material, such as a first oligonucleotide (e.g., primer) coupled thereto, and a second bead may comprise a second material, such as a second oligonucleotide (e.g., primer) coupled thereto. The first and second oligonucleotides may be the same or different. For example, the first oligonucleotide (e.g., first primer) may have the same nucleic acid sequence as the second oligonucleotide (e.g., second primer) or a different nucleic acid sequence. In some cases, the first oligonucleotide (e.g., first primer) may comprise a first nucleic acid sequence and a second nucleic acid sequence, and the second oligonucleotide (e.g., second primer) may comprise a third nucleic acid sequence and a fourth nucleic acid sequence. The first and third nucleic acid sequences may be the same. For example, the first and third nucleic acid sequences may be barcode sequences. The second and fourth nucleic acid sequences may be different. For example, the second and fourth nucleic acid sequences may be functional sequences configured to perform different functions. The second and fourth nucleic acid sequences may be primer (e.g., capture) sequences configured to capture different nucleic acid molecules, as described herein. In an example, the first bead may have a plurality of first oligonucleotides (e.g., first primers) coupled thereto and the second bead may have a plurality of second oligonucleotides (e.g., second primers) coupled thereto, where a given first oligonucleotide of the plurality of first oligonucleotides comprises a first nucleic acid sequence and a second nucleic acid sequence and a given second oligonucleotide of the plurality of second oligonucleotides comprises a third nucleic acid sequence and a fourth nucleic acid sequence. The first and third nucleic acid sequences may be the same (e.g., barcode sequences). The second and fourth nucleic acid sequences may be different (e.g., different functional sequences). In some cases, the second nucleic acid sequences of the plurality of first oligonucleotides coupled to the first bead may vary, and/or the fourth nucleic acid sequences of the plurality of first oligonucleotides coupled to the second bead may vary. For example, the second nucleic acid sequences and/or the fourth nucleic acid sequences may be random N-mers that may be suitable for capturing various template nucleic acid molecules. Nucleic acid sequences of oligonucleotides coupled to a bead may have any useful sequence of any useful base composition and length. In some cases, a nucleic acid sequence of an oligonucleotide coupled to a bead may comprise only canonical nucleotides, while in other cases, a nucleic acid sequence of an oligonucleotide coupled to a bead may comprise one or more nucleotide analogs. A nucleic acid sequence may comprise one or more labels or dyes, such as one or more fluorescent labels, dyes, magnetic labels, radiofrequency labels, or other tags. A nucleic acid sequence of an oligonucleotide coupled to a bead may comprise one or more additional features such as a capture moiety (e.g., biotin), a replication block, cleavable base, or reversible terminator.

As used herein, the term “primer” or “primer molecule” generally refers to a polynucleotide which is complementary to a portion of a template nucleic acid molecule. For example, a primer may be complementary to a portion of a strand of a template nucleic acid molecule. The primer may be a strand of nucleic acid that serves as a starting point for nucleic acid synthesis, such as a primer extension reaction which may be a component of a nucleic acid reaction (e.g., nucleic acid amplification reaction such as PCR). A primer may hybridize to a template strand and nucleotides (e.g., canonical nucleotides or nucleotide analogs) may then be added to the end(s) of a primer, sometimes with the aid of a polymerizing enzyme such as a polymerase. Thus, during replication of a DNA sample, an enzyme that catalyzes replication may start replication at the 3′-end of a primer attached to the DNA sample and copy the opposite strand. A primer (e.g., oligonucleotide) may have one or more functional groups that may be used to couple the primer to a support or carrier, such as a bead or particle.

A primer may be completely or partially complementary to a template nucleic acid. A primer may exhibit sequence identity or homology or complementarity to the template nucleic acid. The homology or sequence identity or complementarity between the primer and a template nucleic acid may be based on the length of the primer. For example, if the primer length is about 20 nucleic acids, it may contain 10 or more contiguous nucleic acid bases complementary to the template nucleic acid.

The complementarity or homology or sequence identity between the primer and the template nucleic acid may be limited. The length of the primer may be between 8 nucleotide bases to 50 nucleotide bases. The length of the primer may be more than 2 nucleotide bases, more than 3 nucleotide bases, 4 nucleotide bases, 5 nucleotide bases, 6 nucleotide bases, 7 nucleotide bases, 8 nucleotide bases, 9 nucleotide bases, 10 nucleotide bases, 11 nucleotide bases, 12 nucleotide bases, 13 nucleotide bases, 14 nucleotide bases, 15 nucleotide bases, 16 nucleotide bases, 17 nucleotide bases, 18 nucleotide bases, 19 nucleotide bases, 20 nucleotide bases, 21 nucleotide bases, 22 nucleotide bases, 23 nucleotide bases, 24 nucleotide bases, 25 nucleotide bases, 26 nucleotide bases, 27 nucleotide bases, 28 nucleotide bases, 29 nucleotide bases, 30 nucleotide bases, 31 nucleotide bases, 32 nucleotide bases, 33 nucleotide bases, 34 nucleotide bases, 35 nucleotide bases, 37 nucleotide bases, 40 nucleotide bases, 42 nucleotide bases, 45 nucleotide bases, 47 nucleotide bases or 50 nucleotide bases. The length of the primer may be less than 50 nucleotide bases, 47 nucleotide bases, 45 nucleotide bases, 42 nucleotide bases, 40 nucleotide bases, 37 nucleotide bases, 35 nucleotide bases, 34 nucleotide bases, 33 nucleotide bases, 32 nucleotide bases, 31 nucleotide bases, 30 nucleotide bases, 29 nucleotide bases, 28 nucleotide bases, 27 nucleotide bases, 26 nucleotide bases, 25 nucleotide bases, 24 nucleotide bases, 23 nucleotide bases, 22 nucleotide bases, 21 nucleotide bases, 20 nucleotide bases, 19 nucleotide bases, 18 nucleotide bases, 17 nucleotide bases, 16 nucleotide bases, 15 nucleotide bases, 14 nucleotide bases, 13 nucleotide bases, 12 nucleotide bases, 11 nucleotide bases, 10 nucleotide bases, 9 nucleotide bases, 8 nucleotide bases, 7 nucleotide bases, 6 nucleotide bases, 5 nucleotide bases, 4 nucleotide bases, 3 nucleotide bases or 2 nucleotide bases.

The term “% sequence identity” may be used interchangeably herein with the term “% identity” and may refer to the level of nucleotide sequence identity between two or more nucleotide sequences, when aligned using a sequence alignment program. As used herein, 80% identity may be the same thing as 80% sequence identity determined by a defined algorithm and means that a given sequence is at least 80% identical to another length of another sequence. The % identity may be selected from, e.g., at least 60%, at least 65%, at least 70%, at least 75%, at least 80%, at least 85%, at least 90%, at least 95%, or at least 99% or more sequence identity to a given sequence. The % identity may be in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” may be used interchangeably herein with the terms “% homology,” “% sequence identity,” or “% identity” and may refer to the level of nucleotide sequence homology between two or more nucleotide sequences, when aligned using a sequence alignment program. For example, as used herein, 80% homology may be the same thing as 80% sequence homology determined by a defined algorithm, and accordingly a homologue of a given sequence has greater than 80% sequence homology over a length of the given sequence. The % homology may be selected from, e.g., at least 60%, at least 65%, at least 70%, at least 75%, at least 80%, at least 85%, at least 90%, at least 95%, or at least 99% or more sequence homology to a given sequence. The % homology may be in the range of, e.g., about 60% to about 70%, about 70% to about 80%, about 80% to about 85%, about 85% to about 90%, about 90% to about 95%, or about 95% to about 99%.

As used herein, the term “primer extension reaction” generally refers to the binding of a primer to a strand of the template nucleic acid, followed by elongation of the primer(s). It may also include, denaturing of a double-stranded nucleic acid and the binding of a primer strand to either one or both of the denatured template nucleic acid strands, followed by elongation of the primer(s). Primer extension reactions may be used to incorporate nucleotides or nucleotide analogs to a primer in template-directed fashion by using enzymes (e.g., polymerizing enzymes such as polymerases). A primer extension reaction may be a process of a nucleic acid amplification reaction.

The term “adapter” as used herein, generally refers to a molecule (e.g., polynucleotide) that is adapted to permit a sequencing instrument to sequence a target polynucleotide, such as by interacting with a target nucleic acid molecule to facilitate sequencing (e.g., next generation sequencing (NGS)). The sequencing adapter may permit the target nucleic acid molecule to be sequenced by the sequencing instrument. For instance, the sequencing adapter may comprise a nucleotide sequence that hybridizes or binds to a capture polynucleotide attached to a solid support of a sequencing system, such as a bead or a flow cell. The sequencing adapter may comprise a nucleotide sequence that hybridizes or binds to a polynucleotide to generate a hairpin loop, which permits the target polynucleotide to be sequenced by a sequencing system. The sequencing adapter may include a sequencer motif, which may be a nucleotide sequence that is complementary to a flow cell sequence of another molecule (e.g., a polynucleotide) and usable by the sequencing system to sequence the target polynucleotide. The sequencer motif may also include a primer sequence for use in sequencing, such as sequencing by synthesis. The sequencer motif may include the sequence(s) for coupling a library adapter to a sequencing system and sequence the target polynucleotide (e.g., a sample nucleic acid). An adapter may have a first sub-part and a second sub-part. The first sub-part and the second sub-part may have sequence complementarity. An adapter as described herein may be a paired-end adapter useful for generating paired-end sequence reads.

The terms “polymerase,” “polymerizing enzyme, or “polymerization enzyme,” as used herein, generally refer to any enzyme capable of catalyzing a polymerization reaction and may be used interchangeably. A polymerizing enzyme may be used to extend primers with the incorporation of nucleotides or nucleotide analogs. Examples of polymerases include, without limitation, a nucleic acid polymerase. The polymerase may be naturally occurring or synthesized. An example polymerase is a Φ29 polymerase or derivative thereof. A polymerase may be a polymerization enzyme. A transcriptase or a ligase may also be used (i.e., enzymes which catalyze the formation of a bond). Examples of polymerases include a DNA polymerase, an RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poc polymerase, Pab polymerase, Mth polymerase ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, PfuTurbo polymerase, Pyrobest polymerase, KOD polymerase, Bst polymerase, Sac polymerase, Klenow fragment polymerase with 3′ to 5′ exonuclease activity, and variants, modified products and derivatives thereof. The polymerase may be a single subunit polymerase. The polymerase may have high processivity, namely the capability of the polymerase to consecutively incorporate nucleotides in a nucleic acid template without releasing the nucleic acid template.

The term “at least partially” as used herein, generally refers to any fraction of a whole amount. For example, “at least partially” may refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 99.9% of a whole amount.

The term “barcode” or “barcode sequence,” as used herein, generally refers to one or more nucleotide sequences that may be used to identify one or more particular nucleic acids. A barcode may comprise at least 1, 2, 3, 4, 5, 6, 7, 8, 9, 10, 11, 12, 13, 14, 15, 16, 17, 18, 19, 20 or more nucleotides (e.g., consecutive nucleotides). A barcode may comprise at least about 10, about 20, about 30, about 40, about 50, about 60, about 70, about 80, about 90, about 100 or more consecutive nucleotides. All of the barcodes used for an amplification and/or sequencing process (e.g., NGS) may be different. The diversity of different barcodes in a population of nucleic acids comprising barcodes may be randomly generated or non-randomly generated.

A barcode may be comprised of one or more segments. For example, a barcode may comprise a first segment that has a first nucleic acid sequence and a second segment that has a second nucleic acid sequence. The first nucleic acid sequence may be the same or different than the second nucleic acid sequence. Barcode sequences comprising multiple segments may be assembled in a combinatorial fashion according to a split-pool scheme, in which a plurality of different first segments are distributed amongst a plurality of first partitions, the contents which are then pooled and distributed amongst a plurality of second partitions. A plurality of different second segments are then distributed amongst the plurality of second partitions and linked to the plurality of different first segments within the plurality of second partitions, and then the contents of the plurality of second partitions are pooled. The process may be repeated any number of times using any number of different segments and partitions to provide any level of barcode diversity. In some cases, the first segment of a barcode sequence may be coupled to a bead.

As described herein, the use of barcodes may permit high-throughput analysis of multiple samples using next generation sequencing techniques. Multiple samples or multiple portions of a sample may be barcoded. In an example, a sample comprising a plurality of nucleic acid molecules may be distributed throughout a plurality of partitions (e.g., droplets in an emulsion), where each partition comprises a nucleic acid barcode molecule comprising a unique barcode sequence. The sample may be partitioned such that all or a majority of the partitions of the plurality of partitions include at least one nucleic acid molecule of the plurality of nucleic acid molecules. A nucleic acid molecule and nucleic acid barcode molecule of a given partition may then be used to generate one or more copies and/or complements of at least a sequence of the nucleic acid molecule (e.g., via nucleic acid amplification reactions), which copies and/or complements comprise the barcode sequence of the nucleic acid barcode molecule or a complement thereof. The contents of the various partitions (e.g., amplification products or derivatives thereof) may then be pooled and subjected to sequencing. In some cases, nucleic acid barcode molecules may be coupled to beads. In such cases, the copies and/or complements may also be coupled to the beads. Nucleic acid barcode molecules, and copies and/or complements may be released from the beads within the partitions or after pooling to facilitate nucleic acid sequencing using a sequencing instrument. Because copies and/or complements of the nucleic acid molecules of the plurality of nucleic acid molecules each include a unique barcode sequence or complement thereof, sequencing reads obtained using a nucleic acid sequencing assay may be associated with the nucleic acid molecule of the plurality of nucleic acid molecules to which they correspond. This method may be applied to nucleic acid molecules included within cells divided amongst a plurality of partitions, and/or nucleic acid molecules deriving from a plurality of different samples. Alternatively, a sample comprising a plurality of nucleic acid molecules may be barcoded without the use of partitions. For example, different nucleic acid molecules may be immobilized to different beads within an open reaction space or bulk reaction mixture, wherein each bead comprises a different barcode sequence (e.g., unique bead species).

In some aspects, provided herein are systems, methods, and compositions wherein a reaction space, reaction mixture, or partition comprises more than a single bead. In some aspects, provided herein are systems, methods, and compositions wherein a reaction space, reaction mixture, or partition comprises more than a single analyte (e.g., nucleic acid molecule, e.g., template nucleic acid molecule). For example, the reaction space may comprise an open substrate wherein multiple locations on the reaction space have common fluidic access to a solution (e.g., reaction mixture). Beneficially, the systems, methods, and compositions of the present disclosure need not depend on singular loading of content (e.g., with single bead, with single analyte) for successful downstream processing. Beneficially, the systems, methods, and compositions of the present disclosure need not depend on forming partitions that are at most singularly loaded (e.g., with single bead, with single analyte) according to the Poisson distribution, which can often lead to a waste of resources where a substantial number of partitions consume certain resources (e.g., bead, analyte, reagent, etc.) but are not useful because of a lack of (or otherwise wrong number or wrong composition of) one or more of certain other resources (e.g., bead, analyte). Beneficially, the systems, methods, and compositions may obviate partitioning. In some instances, beneficially, the systems, methods, and compositions of the present disclosure may achieve higher efficiency and/or higher output than systems, methods, and compositions that depend on singular loading.

### I. Unique Support Species

The present disclosure provides methods for analyzing and/or processing a biological sample (e.g., biological sample or cell-free biological sample). In particular, the present disclosure provides a method for analyzing and/or processing a nucleic acid sample comprising one or more template nucleic acid molecules (e.g., a plurality of nucleic acid molecules), such as a plurality of deoxyribonucleic acid (DNA) and/or ribonucleic acid (RNA) molecules.

For template nucleic acid molecule amplification and analysis, provided herein are compositions and methods that can comprise a plurality of unique support species. As used herein, the term “template nucleic acid molecule” may generally refer to a nucleic acid molecule from a biological sample to be analyzed, or derivative thereof (e.g., an original targeted molecule, an original non-targeted molecule, an amplicon, a complement, an amplicon of the complement, a double-stranded form thereof, a single-stranded form thereof, etc.). The template nucleic acid molecule may comprise a sequence corresponding to a template nucleic acid sequence (e.g., identical to, complement thereof). Each support species of such plurality of unique support species can be configured to capture a template nucleic acid molecule of a plurality of template nucleic acid molecule. The plurality of unique support species can be a plurality of unique particle species. The plurality of unique particle species can be a plurality of unique bead species (also referred to herein “bead species”). A population of supports may comprise any number of bead species sub-populations, such as a first bead species sub-population, a second bead species sub-population, etc. Beads within each sub-population may be of the same bead species. In some instances, the plurality of unique support species can comprise a first support (of a first support species) and a second support (of a second support species). Such first support can comprise a first plurality of nucleic acid molecules, and the second support can comprise a second plurality of nucleic acid molecules. In some instances, each nucleic acid molecule of the first plurality of nucleic acid molecules comprises or consists of an identical nucleic acid sequence, and each nucleic acid molecule of the second plurality of nucleic acid molecules comprises or consists of an identical nucleic acid sequence, and wherein the nucleic acid molecules of the first and second support comprise or consist of different nucleic acid sequences. In some instances, the first plurality of nucleic acid molecules can be a plurality of first primer molecules, and the second plurality of nucleic acid molecules can be a plurality of second primer molecules.

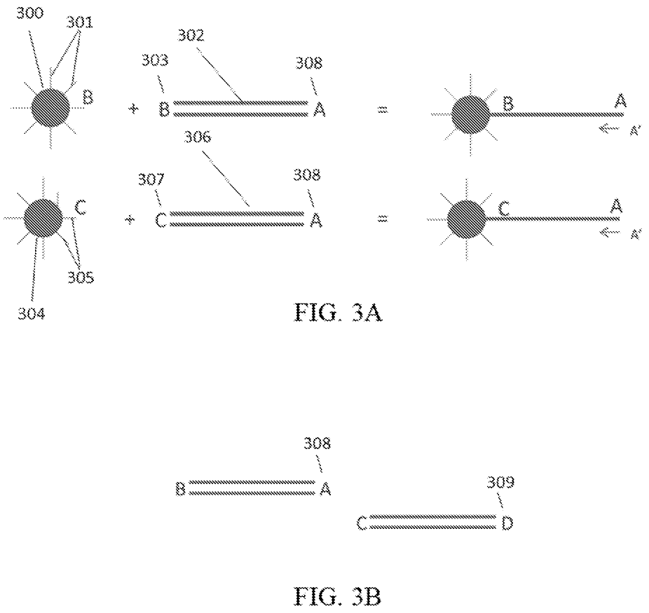

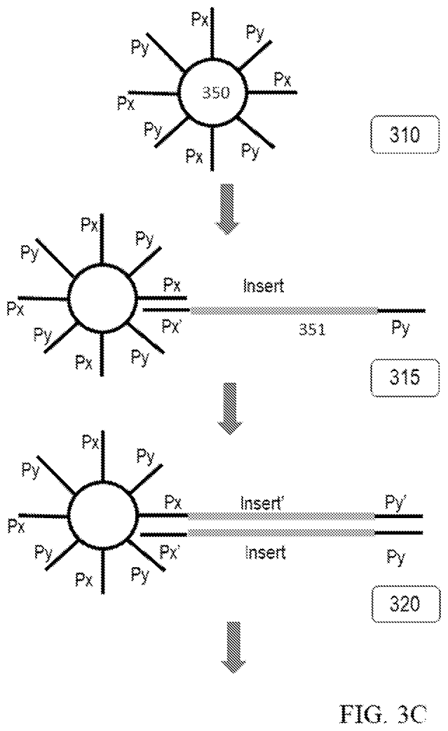

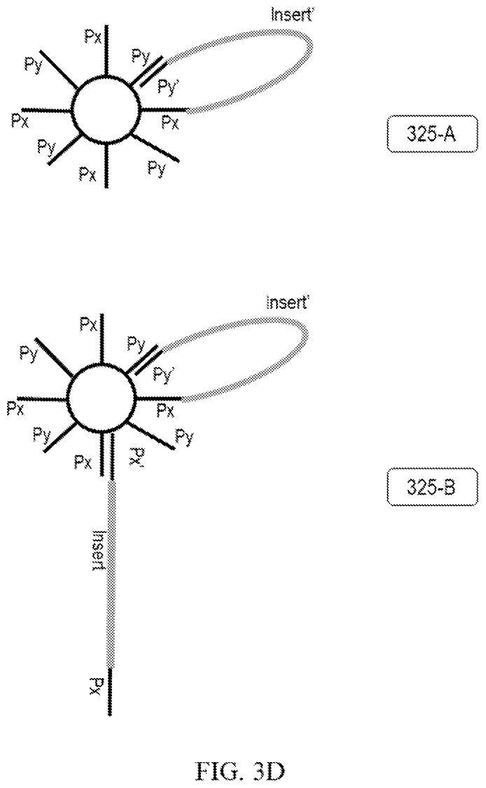

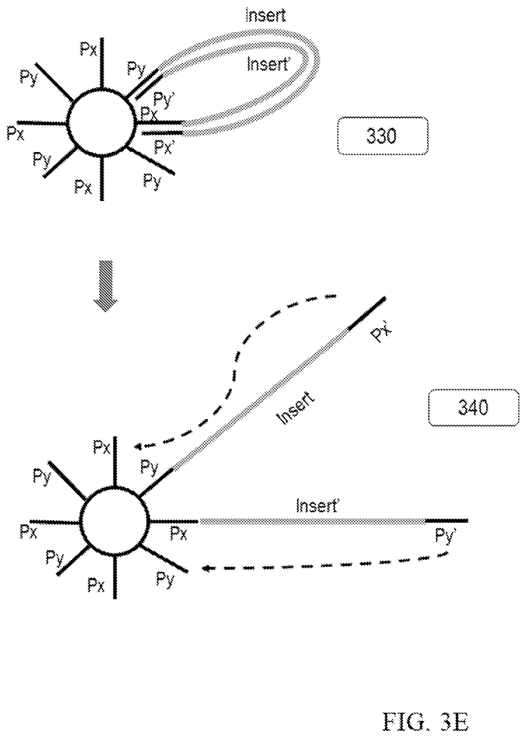

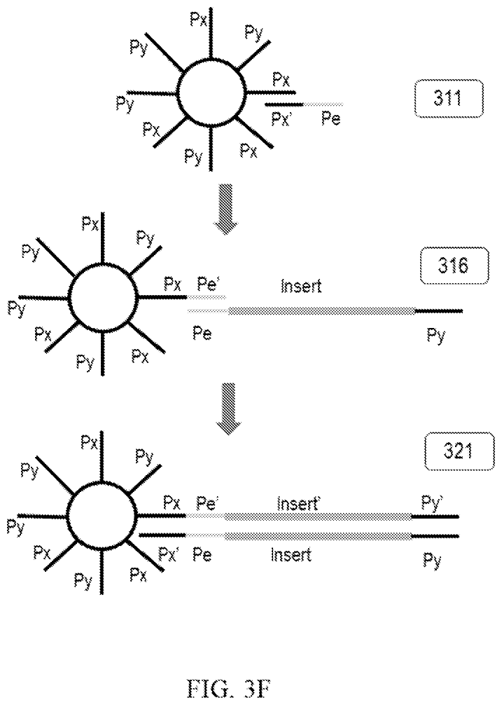

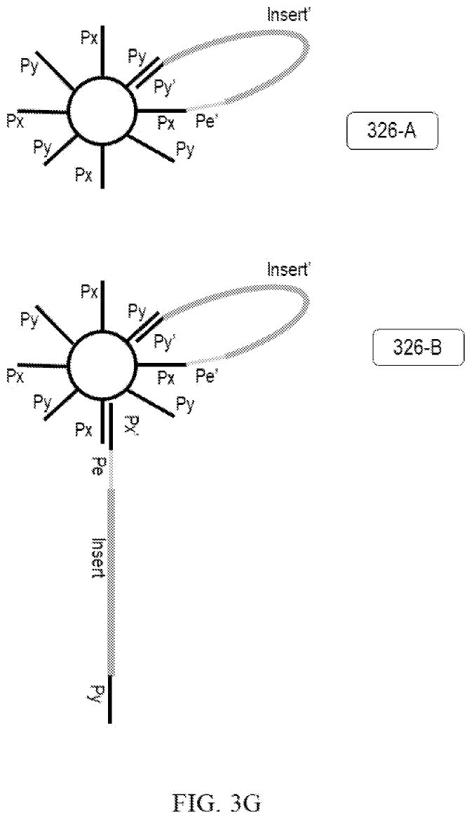

Referring to FIG. 3A, a first plurality of primer molecules 301 can be attached to the first support 300 (e.g., a first bead) of the plurality of supports and can be configured to hybridize with a first template nucleic acid molecule 302 of the plurality of template nucleic acid molecules, or a portion thereof. Such portion of the first template nucleic acid molecule can be a first adapter sequence 303. Similarly, a second plurality of primer molecules 305 can be attached to a second support 304 of the plurality of supports and can be configured to hybridize with a second template nucleic acid molecule 306 of the plurality of template nucleic acid molecules, or a portion thereof. Such portion of the second template nucleic acid molecule can be a second adapter sequence 307. The nucleic acid sequence of the first primer molecule of the first support can be different than the nucleic acid sequence of the second primer molecule of the second support. The nucleic acid sequence of the first adapter sequence can be different to the nucleic acid sequence of the second adapter sequence. In other words, and as illustrated in FIG. 3A, the nucleic acid sequence of a first primer molecule 301 may not be complementary to the nucleic acid sequence of a second adapter sequence 307, and the nucleic acid sequence of a second primer molecule 305 may not be complementary to the nucleic acid sequence of a first adapter sequence 303. Thus, each unique support (e.g., bead) can be capable of immobilizing only the template nucleic acid molecule comprising an adapter sequence that is complementary to the primer sequence of the of the support.

A plurality of first primer molecules, e.g., 301, can be covalently or non-covalently attached to the surface of a first unique support, e.g., 300. Covalent attachment can be provided using, e.g., biotin-streptavidin, azide-cyclooctyne, thiol-maleimide, tetrazine-trans-cyclooctene, or other (e.g., biorthogonal) bioconjugation methods. Non-covalent attachment can be provided using, e.g., hybridization of a first portion of a first primer molecule with a complementary nucleic acid molecule already attached to the unique support or using ionic interactions.

In some instances, the plurality of unique support species (e.g., bead species) can be part of a solution or suspension. Such solution can be a bulk solution. Such solution or suspension can further comprise the plurality of template nucleic acid molecules comprising the first and second template nucleic acid molecules. In various cases, the solution or suspension can further comprise one or more reagents. Such one or more reagents can comprise enzymes, nucleotides, ions, etc. In some cases, such one or more reagents can comprise enzymes, nucleotides, primers, and other components and building blocks for nucleic acid extension and/or amplification. Thus, the plurality of unique support species can be in fluidic contact with the plurality of template nucleic acid molecules and the one or more reagents. It can be appreciated that the solution comprising the plurality of unique support species may be contained or defined by any reaction space. For example, the solution may be contained in any container or reaction vessel. For example, the solution may be contained in a droplet within an emulsion, such as prior to or during performance of emulsion PCR (ePCR).

In some instances, the plurality of unique support species can be immobilized in a reaction space that is in fluidic contact with a solution comprising the plurality of template nucleic acid molecules. The solution can comprise the one or more reagents.

Thus, in some instances, the methods provided herein can comprise using such one or more reagents to generate extension products of the template nucleic acid molecules that are immobilized on the plurality of unique support species (e.g., a unique support can have one template nucleic acid molecule attached thereto). Referring back to FIG. 3A, in some cases, the respective distal adapter sequences 308 located at the distal end of the different template nucleic acid molecules (relative to the supports) can comprise or consist of identical nucleic acid sequences. Primer molecules from the solution can hybridize to such distal adapters and allow the generation of extension products. In other instances, and as illustrated in FIG. 3B, each template nucleic acid molecule of a plurality of template nucleic acid molecules may not only have a unique proximal adapter sequence (e.g., 303, 307) but also a unique distal adapter sequence (e.g., 308, 309). Using such unique distal adapters may ensure that amplification of a first template nucleic acid molecule does not outcompete amplification of a second template nucleic acid molecule.

The one or more reagents can also be used to generate a plurality of template nucleic acid amplification products. Such plurality of template nucleic acid amplification products can comprise a plurality of amplification products of a first template nucleic acid molecule and a plurality of amplification products of a second template nucleic acid molecule. Each amplification product of the first and second template nucleic acid molecules can comprise a nucleic acid sequence that corresponds to the first and second template nucleic acid molecule sequences, respectively. The amplification products of a first template nucleic acid molecule immobilized on a first unique support (e.g., bead) can hybridize with additional first primer molecules (e.g., 301) located on the surface of that first unique support and hence be immobilized by the same support, thereby generating a monoclonal support (e.g., support comprising a monoclonal population). Such method can be performed using a plurality of unique supports (e.g., beads) and hence generate a plurality of monoclonal supports, wherein each monoclonal support comprises a single template nucleic acid molecule (from the plurality of template nucleic acid molecules) and amplicons thereof attached to its surface.

In other instances herein, amplification of a plurality of template nucleic acid molecules can be performed prior to immobilization of the plurality of template nucleic acid molecules, and amplicons thereof, to a plurality of unique support species (e.g., beads), to generate a plurality of monoclonal supports.

It can be appreciated that, in various cases, not all amplification products (e.g., only a subset of amplification products) of a given template nucleic acid molecule are attached to a given unique support. Similarly, in some cases, not all primer molecules of a given unique support are hybridized to a template nucleic acid molecule or amplicon thereof, thus resulting in a number of unreacted primers. In other cases, not all unique supports may immobilize a template nucleic acid or amplicons thereof, resulting in “empty” supports (e.g., negative supports) comprising only unreacted or non-amplified primers. Methods of removing such unreacted or non-amplified primers from a mixture prior to assaying or analyzing (e.g., sequencing) are described elsewhere herein and may allow for significantly increased sensitivity during analysis.

Amplification of template nucleic acid molecules herein can comprise performing recombinase polymerase amplification (RPA). In such cases, the one or more reagents can comprise one or more recombinase enzymes. The one or more reagents can also include a binding protein. Such binding protein can be a single-stranded DNA binding protein. In some cases, the one or more reagents can further comprise a crowding agent or alternative, such as a polyethylene glycol (PEG), or a derivative thereof. Thus, in some instances, the one or more reagents can comprise one or more of a recombinase enzyme, a binding protein, and a crowding agent to allow for, e.g., RPA. During amplification, such as via RPA or other amplification method, even as multiple unique supports are in fluidic contact with the same solution, some reagents (e.g., crowding agent or alternative) may help prevent migration of amplification products generated in solution near an immediate vicinity of a first unique support to a neighboring or other unique support that can immobilize such amplification products.

FIGS. 3C-3G illustrate a method for on-support bridge amplification. With reference to FIG. 3C, a support 350 may be provided (310). The support may comprise two species of primer molecules, Px and Py. A template nucleic acid molecule 351 may be provided, wherein the template nucleic acid molecule comprises a first adapter sequence (Px′) complementary to a first species of primer (Px) on the support, an insert sequence (e.g., template sequence), and a second adapter sequence (Py) corresponding to a second species of primer (Py) on the support. For example, the adapter sequences may be ligated to the insert sequence. The template nucleic acid molecule may anneal (315) to the support 350 via hybridization of the first adapter sequence to a first primer species (Px) molecule, and the first primer species molecule may subsequently be extended (320) to generate an extended molecule comprising a sequence (insert′) complementary to the insert sequence and a sequence (Py′) complementary to the second primer species (Py) on the support. With reference to FIG. 3D, the template nucleic acid molecule may be denatured and removed from the support, and the free end of the extended molecule may anneal (325-A) to the support 350 via hybridization of the sequence (Py′) complementary to the second primer species (Py) to a second primer species (Py) molecule on the support. The template nucleic acid molecule may be washed. In some cases, for example when the reaction takes place in a contained reaction vessel (e.g., droplet during ePCR), the template nucleic acid molecule removed from the support or copy thereof may anneal (325-B) to another first primer species (Px) molecule on the support 350. With reference to FIG. 3E, subsequent to annealing of the second primer species (Py) molecule on the support with the extended molecule, the second primer species molecule may be extended to generate a second extended molecule comprising a sequence corresponding to the insert sequence and a sequence (Px′) complementary to the first species of primer (Px) on the support. The extended molecule and the second extended molecule may be denatured (340) such that a reverse strand and a forward strand are each immobilized on the support via the first primer species molecule and the second primer species molecule. Such strands may each continue to facilitate cycles of annealing-extending-denaturing using other primer species molecules on the support to generate multiple copies of the reverse strands (comprising the insert′ sequence) and multiple copies of the forward strands (comprising the insert sequence) immobilized to the support.

With reference to FIG. 3F, in some cases, an extension primer may be provided, wherein the extension primer comprises a first extension sequence (Pe) and a sequence (Px′) complementary to a first species of primer (Px) on the support. The extension primer may be annealed (311) to the support via hybridization of the sequence (Px′) complementary to the first species of primer to a first primer species (Px) molecule, and the first primer species molecule may be subsequently extended to generate an extended primer comprising a sequence (Pe′) complementary to the first extension sequence (Pe). A template nucleic acid molecule may be provided, wherein the template nucleic acid molecule comprises a sequence (Pe) corresponding to the first extension sequence, an inset sequence, and a sequence (Py) corresponding to a second species of primer on the support. The template nucleic acid molecule may be annealed (316) to the support via hybridization of the extended primer (comprising the Pe′ sequence) to the sequence (Pe) corresponding to the first extension sequence, and the extended primer may subsequently be extended (321) to generate an extended molecule comprising a sequence (insert′) complementary to the insert sequence and a sequence (Py′) complementary to the second primer species (Py) on the support. The template nucleic acid molecule may be extended to comprise a sequence (Px′) complementary to the first species of primer (Px) on the support. With reference to FIG. 3G, the extended template nucleic acid molecule may be denatured and removed from the support, and the free end of the extended molecule may anneal (326-A) to the support via hybridization of the sequence (Py′) complementary to the second primer species (Py) to a second primer species (Py) molecule on the support. The extended template nucleic acid molecule may be washed. In some cases, for example when the reaction takes place in a contained reaction vessel (e.g., droplet during ePCR), the extended template nucleic acid molecule removed from the support or copy thereof may anneal (326-B) to another first primer species (Px) molecule on the support. Thereafter, the second primer species molecule may be extended to generate a second extended molecule. The extended molecule and the second extended molecule may be denatured such that a reverse strand and a forward strand are each immobilized on the support via the first primer species molecule and the second primer species molecule. Such strands may each continue to facilitate cycles of annealing-extending-denaturing using other primer species molecules on the support to generate multiple copies of the reverse strands (comprising the insert′ sequence) and multiple copies of the forward strands (comprising the insert sequence) immobilized to the support.

It will be appreciated that a plurality of supports may be subject to different extension primers which comprise different extension sequences to generate unique support species as described elsewhere herein. For example, a first support species may be subject to a first extension primer comprising a first extension sequence (Pe) to generate a first extended primer comprising a sequence (Pe′) complementary to the first extension sequence, and a second support species may be subject to a second extension primer comprising a second extension sequence (Pg) different from the first extension sequence to generate a second extended primer comprising a sequence (Pg′) complementary to the second extension sequence, such that a first template nucleic acid molecule comprising an adapter sequence corresponding to a given extension sequence (e.g., Pe) may be capable of annealing to the first support species but not to the second support species to initiate the bridge amplification reaction.

It will also be appreciated that, regardless of whether extension primers are used, a plurality of supports may comprise unique support species as described elsewhere herein. A first unique support species may comprise a first pair of primer species (e.g., Px and Py) and a second unique support species may comprise a second pair of primer species (e.g., Pm and Pn) different form the first pair of primer species, such that a first template nucleic acid molecule comprising an adapter sequence complementary to a given primer sequence (e.g., Px′ or Py′) may be capable of annealing to the first unique support species but not to the second unique support species. Beneficially, a plurality of unique supports, comprising distinct primer species, and a plurality of template nucleic acid molecules, comprising distinct adapter sequences, may be provided in a common reaction space with fluidic access between the different reagents, and the resulting positive beads may each correspond only to a single template nucleic acid molecule with monoclonal or substantially monoclonal populations of the forward strand and the reverse strand of the single template nucleic acid molecule.

In some embodiments, the systems and methods disclosed herein can include supports that comprise two, three, four, five, six, seven, eight, nine, ten unique support species. Each unique support species can comprise a pair of primer species that allows selective interactions between the respective support species with an intended binding partner (e.g., a complementary nucleic acid sequence within an adapter region of a sample nucleic acid or an extension primer sequence which can subsequently bind to a complementary nucleic acid sequence within an adapter region of a sample nucleic acid). In some embodiments, the systems and methods disclosed herein can include supports that comprise more than ten unique support species. Each unique support species can comprise a pair of primer species that allows selective interactions between the respective support species with an intended binding partner (e.g., a complementary nucleic acid sequence within an adapter region of a sample nucleic acid or an extension primer sequence which can subsequently bind to a complementary nucleic acid sequence within an adapter region of a sample nucleic acid).

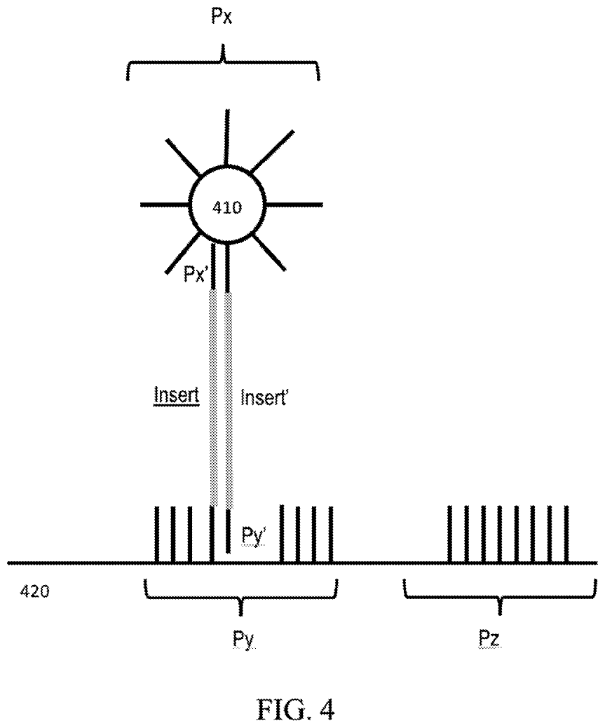

FIG. 4 illustrates another embodiment of on surface amplification. A support 410 may be provided, wherein the support comprises a first primer species (Px). A surface 420 may be provided, wherein the surface is independent of the support and comprises one or more additional primer species (Py, Pz). A template nucleic acid molecule may be provided, wherein the template nucleic acid molecule comprises a first adapter sequence (Px′) complementary to a first species of primer (Px) on the support, an insert sequence (e.g., template sequence), and a second adapter sequence (Py) corresponding to a second species of primer (Py) on the surface. For example, the adapter sequences may be ligated to the insert sequence. The template nucleic acid molecule may anneal to the support via hybridization of the first adapter sequence to a first primer species (Px) molecule, and the first primer species molecule may subsequently be extended to generate an extended molecule comprising a sequence (insert′) complementary to the insert sequence and a sequence (Py′) complementary to the second primer species (Py) on the support. The template nucleic acid molecule may be denatured and removed from the support, and the free end of the extended molecule may anneal to the surface via hybridization of the sequence (Py′) complementary to the second primer species (Py) to a second primer species (Py) molecule on the surface, as shown in FIG. 4. Subsequent to annealing of the second primer species (Py) molecule on the surface with the extended molecule, the second primer species molecule may be extended to generate a second extended molecule immobilized to the surface comprising a sequence corresponding to the insert sequence and a sequence (Px′) complementary to the first species of primer (Px) on the support. The extended molecule and the second extended molecule may be denatured such that a reverse strand is immobilized to the support via the first primer species molecule and a forward strand is immobilized on the surface via the second primer species molecule. Such strands may each continue to facilitate cycles of annealing-extending-denaturing, for example by the reverse strand immobilized to the support annealing to other second primer species molecules on the surface and/or by the forward strand immobilized to the surface annealing to other first primer species molecules on the support, to generate multiple copies of the reverse strands (comprising the insert′ sequence) on the support and multiple copies of the forward strands (comprising the insert sequence) immobilized to the surface. It will be appreciated that the surface may comprise a plurality of different primer species each configured to facilitate amplification of different template nucleic acid molecules on different support species (e.g., which comprise distinct primer species).

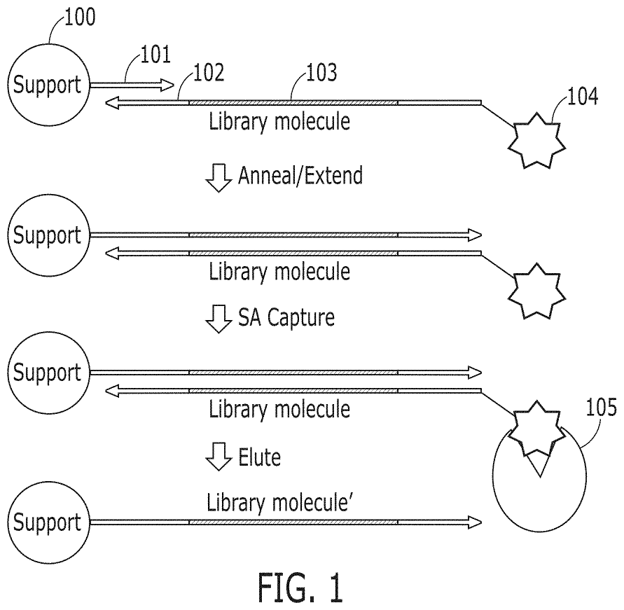

A plurality of unique supports (e.g., beads), or a subset thereof, can be immobilized to a surface of a substrate. The substrate can have a planar surface. In some cases, such substrate can comprise or consist of a substantially planar array. In cases in which at least a subset of unique supports of the plurality of unique supports has a template molecule attached to its surface, the plurality of unique supports can be immobilized to the surface before or after amplification of the template nucleic acid molecules. In some cases, the plurality of unique supports is immobilized to the surface before amplification of the template nucleic acid molecules. In other cases, the plurality of unique supports is immobilized to the surface after amplification of the template nucleic acid molecules. A unique support can be immobilized to a surface using a variety of chemistries, e.g., bioconjugation chemistries as described elsewhere herein. Referring to FIG. 1, a template nucleic acid molecule 103 can be immobilized on a unique support 100 (e.g., bead) via hybridization of a primer molecule 101 with a first adapter sequence 102 of the template nucleic acid molecule 103. Following extension, and either before or after amplification of the template molecule, the unique support can be immobilized to a surface via interaction of a capture moiety 104 (e.g., biotin) of the template nucleic acid molecule and a capturing moiety 105 (e.g., streptavidin) attached to the substrate surface. The capture moiety and capturing moiety may be any capturing pair described herein.

Provided herein are support compositions. A composition can comprise a mixture comprising a plurality of supports, wherein the plurality of supports comprises: (i) a first support comprising, immobilized thereto, a first primer of a plurality of first primers configured to hybridize to a first exogenous adapter sequence, and (ii) a second support comprising, immobilized thereto, a second primer of a plurality of second primers configured to hybridize to a second exogenous adapter sequence, wherein the first exogenous adapter sequence and the second exogenous adapter sequence comprise different sequences, and wherein the first primer and the second primer comprise different sequences.

An exogenous adapter sequence may comprise a sequence that does not originate from a biological sample (e.g., cell, subject, etc.), and/or may be a sequence sourced external to the biological sample (e.g., engineered or synthesized, originating from another biological sample, etc.) that is introduced to the biological sample. For example, an exogenous adapter sequence may be ligated to a template nucleic acid molecule.

The plurality of supports may be a plurality of particles, such as a plurality of beads.

In some instances, the plurality of supports can be immobilized to a substrate surface. The plurality of supports can be immobilized to the substrate surface via any interactions, such as via electrostatic interactions, or via hybridization of one or more oligonucleotide molecules.

The plurality of supports may be provided in a solution. The solution may comprise one or more reagents described herein. For example, the reagents can comprise one or more additional primers, enzymes, proteins, crowding agents, buffers, cations, or a combination thereof.

In some instances, the mixture may comprise a plurality of partitions, such as a plurality of droplets or a plurality of wells.

In some instances, the first primer can comprise a first extended primer region that is absent from other first primers in the plurality of first primers and the second primer can comprise a second extended primer region that is absent from other second primers in the plurality of second primers. The first nucleic acid molecule can be immobilized to the first support via the first extended primer region and the second nucleic acid molecule can be immobilized to the second support via the second extended primer region.

In some instances, each first primer of the plurality of first primers can be identical (e.g., comprise identical sequences). Each second primer of the plurality of second primers can be identical (e.g., comprise identical sequences).

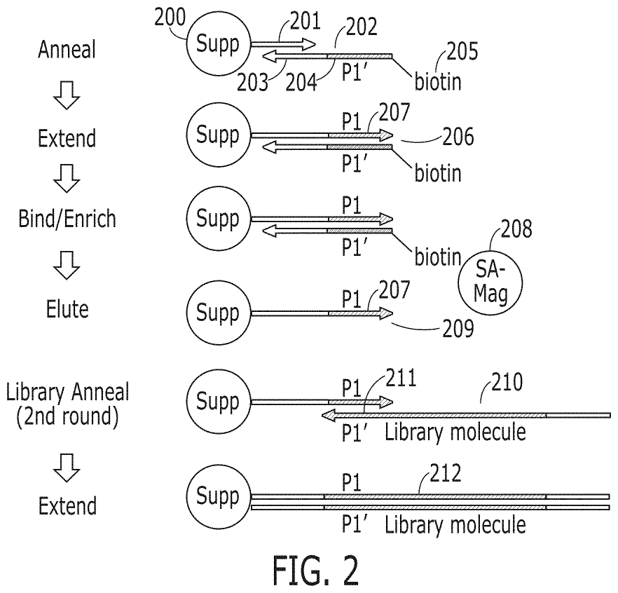

As described above, provided herein are compositions and methods comprising unique supports, i.e., supports that each comprise one species of primer molecules (e.g., primer molecules with identical nucleic acid sequences). In some instances, a unique support can comprise a plurality of unique extended primer molecules. Thus, provided herein is a first unique support of a plurality of unique supports comprising a first primer molecule (e.g., 207 of FIG. 2), a plurality of first support primer molecules (e.g., 201), and a first extension primer molecule (e.g., 209=201+207). Such first extension primer molecule can comprise a first support primer molecule of the plurality of first support primer molecules and the first primer molecule. Each first support primer molecule of the plurality of first support primer molecules of a first support (e.g., bead) can comprise or consist of an identical nucleotide sequence. In some cases, the first extension primer molecule is configured to hybridize with a first template nucleic acid molecule (e.g., 202), or a portion thereof, wherein such portion can be a first adapter sequence (e.g., 203). Similarly, a second support of a plurality of supports can comprise a second primer molecule, a plurality of second support primer molecules, and a second extension primer molecule, wherein such second extension primer molecule comprises a second support primer molecule of the plurality of second support primer molecules and the second primer molecule. Each second support primer molecule of the plurality of second support primer molecules can comprise or consist of an identical nucleotide sequence. In some cases, the second extension primer molecule is configured to hybridize with a second template nucleic acid molecule, or a portion thereof, wherein such portion can be a second adapter sequence. The first extension primer molecule of the first support may not be complementary to, and thus may not hybridize with, the second template nucleic acid sequence, or the portion thereof. Similarly, the second extension primer molecule of the second support may not be complementary to, and thus may not hybridize with, the first template nucleic acid sequence, or the portion thereof.

Unique support species can provide numerous advantages for processes that involve partitioning, and for processes that do not involve partitioning. During partitioning workflows (e.g., ePCR workflows), for example, a population of beads and a population of template molecules may be partitioned into a plurality of partitions, the desirous partition having a single bead and a single template molecule. However, due to Poisson distribution, usually only a small population of partitions end up having a single bead and a single template molecule. Where a partition has no template, the resulting bead (or lack thereof) may be ‘negative’ beads which do not produce sequencing reads. Where a partition has no bead, the template molecules, which may be rare, may be wasted. Where a partition has a single bead and multiple template molecules, a resulting amplified bead may comprise a multiclonal population (e.g., mix of the multiple template molecule copies), which may not produce valuable sequencing reads. If beads are overloaded during partitioning, such that it is more likely that a partition with a template molecule has at least one bead (to avoid the scenario where there is a partition with a template and no bead), such that a partition has a plurality of beads and a single template molecule, the resulting amplified beads will produce duplicate sequencing reads. Where a partition has a plurality of beads and a plurality of template molecules, a combination of the above problems may result. Any of the above situations can waste substantial time and resources and decrease process efficiency. Beneficially, a population of unique support species may be partitioned such that statistically it is very unlikely that a partition has multiple beads of the same species. Thus, during subsequent amplification workflows, even where a partition has multiple beads, the risk of producing duplicate reads is substantially lower because the amplicons may only be capable of coupling to one species of bead within the partition, resulting in a single positive bead and one or more negative beads. Such positive beads may be isolated, or enriched, prior to sequencing.

Further, the use of unique support species may obviate the need for partitioning. As described elsewhere herein, a population of unique support species and a population of template molecules may be provided in a solution, or otherwise provided such that the population of unique support species have substantial fluidic contact and access to the population of template molecules. Beneficially, a population of unique support species may be provided such that statistically it is very unlikely that two supports of the same species are in the immediate vicinity, e.g., within migration potential, of each other. Thus, during subsequent amplification workflows, even where an amplified copy in solution that is derived from a template immobilized to a first support has fluidic access to a neighboring support, the risk of the amplified copy migrating to the neighboring support and contaminating its existing colony population or producing a duplicate colony is substantially lower because the amplified copy may only be capable of coupling to the first support (or its species), and not be capable of coupling to neighboring supports (or their species).

### Production of Unique Support Species

Further provided herein are compositions and methods for producing unique support species. In various instances, such unique supports can be unique beads (or unique bead species), wherein each unique bead can comprise a plurality of unique and identical primer molecules, as illustrated, e.g., in FIG. 2.

Methods of producing unique support species (e.g., unique bead species) can comprise providing a plurality of supports, wherein each support 200 of the plurality of supports comprises a nucleic acid molecule 201. Such nucleic acid molecule can be attached to the surface of the support, e.g., by using any of the described bioconjugation strategies described herein. In some cases, the nucleic acid molecules attached to each support of the plurality of supports comprise identical nucleic acid sequences. In other cases, the nucleic acid molecules attached to each support of the plurality of supports comprise different nucleic acid sequences.

A first set of supports of the plurality of supports can be contacted with a first plurality of nucleic acid molecules 202, wherein the nucleic acid molecules attached to the surface of the first set of supports can be configured to hybridize with a first portion 203 of the nucleic acid sequence of the first plurality of nucleic acid molecules. In some cases, the first plurality of nucleic acid molecules can comprise identical nucleic acid sequences, or portions that comprise identical nucleic acid sequences. In other cases, the first plurality of nucleic acid molecules can comprise different nucleic acid sequences, or portions that comprise different nucleic acid sequences. In instances where each molecule in the first plurality of nucleic acid molecules comprises an identical nucleic acid sequence, other subsets of supports, e.g., second, third, and fourth sets of supports, can each be contacted with a respective second, third, and fourth plurality of nucleic acid molecules to obtain a plurality of sets of supports, with each set comprising a respective plurality of identical nucleic acid molecules.

Referring back to the first set of supports that is contacted with a first plurality of nucleic acid molecules, such first plurality of nucleic acid molecules can further comprise a second portion 204. Following hybridization, the nucleic acid molecules can be extended, e.g., as shown in FIG. 2, to generate an extension product 206, e.g., on the surface of the supports (e.g., particles such as beads). Such extension reaction can be used to generate support-bound primer molecules 207 (in FIG. 2 referred to as “P1”). In instances where the first set of supports is contacted with a first plurality of identical nucleic acid molecules, all supports of the first set of supports can comprise identical primers, thereby generating a first set of unique supports. Such unique supports can be unique beads.

In various cases, and in order to enrich for unique supports, the first plurality of nucleic acid molecules can further comprise a first reactive moiety 205 coupled to one (or both) of ends, e.g., the 3′ end, as shown in FIG. 2. The first reactive moiety can be a functional group enabling a bioconjugation reaction with a second reactive moiety. In some cases, the first reactive moiety can be a biotin, an azide, a thiol, a tetrazine, a derivative thereof, or another functional group. The first set of supports comprising the extended double-stranded nucleic acid molecule 206 including the first reactive moiety 205 can be brought into proximity (e.g., into fluidic contact) with a surface, wherein such surface (e.g., a planar surface) can comprise the second reactive moiety, wherein such second reactive moiety can be attached thereto. In some instances, the second reactive moiety can be a streptavidin, a cyclooctyne, a maleimide, or a trans-cyclooctene, a derivative thereof, or another functional group. Referring back to FIG. 2, in some instances, the first reactive moiety is a biotin, and the second reactive moiety is streptavidin (SA, 208). In other instances, the first reactive is an oligonucleotide with a capture sequence, and the second reactive moiety is a complementary sequence to the capture sequence. Thus, the first set of supports can be immobilized to the substrate surface via specific interactions between the biotin and streptavidin moieties. Such immobilization can also allow for enrichment of supports that comprise a single species of unique primers 207. The first reactive moiety and the second reactive moiety pair may be any capturing pair described herein.

In a subsequent step, and after removal of supports that do not comprise a unique primer 207, the enriched unique supports can be detached or eluted from the substrate surface to provide a first set of unique supports comprising a single primer species 209 (e.g., an extended primer). This production process can be performed multiple times to generate a plurality of unique sets of supports comprising a unique primer species. In various cases, such unique sets of supports are unique sets of beads.

As shown in FIG. 2, such set of unique supports comprising a single primer species can be used to hybridize with a single template nucleic acid molecule (e.g., of a library of different template nucleic acid molecules) of a sample via an adapter sequence 211 (in FIG. 2 referred to as “P1′”) of the template nucleic acid molecule that can be configured to hybridize with the support-bound primer 207. Upon hybridization, the nucleic acid molecules can be extended, amplified, and analyzed, e.g., sequenced. For template nucleic acid analysis, using a plurality of unique sets of supports each comprising unique primers can be an alternative approach to using excess amounts of supports that all comprise the same primer.

### Unique Primer Clusters on Surfaces

As described elsewhere herein, it will be appreciated that the systems and methods described herein are not limited to clonal amplification of template nucleic acid molecules on unique supports (e.g., unique bead species) and/or amplification in emulsion droplets. That is, a plurality of unique supports, such as a plurality of unique beads, that are in solution, and wherein each unique support or bead comprises a unique “species” of primer molecule, can be translated to a surface of a substrate. In such instance, a surface, e.g., a planar surface, can be divided into a plurality of individually addressable and spatially separated locations. Each of such individual location can be regarded as a single unique support or bead comprising a plurality of unique, and in some cases identical, primers capable of hybridizing with a specific adapter sequence of a template nucleic acid molecule of a plurality of template nucleic acid molecules of a samples (e.g., a biological sample or a clinical sample).

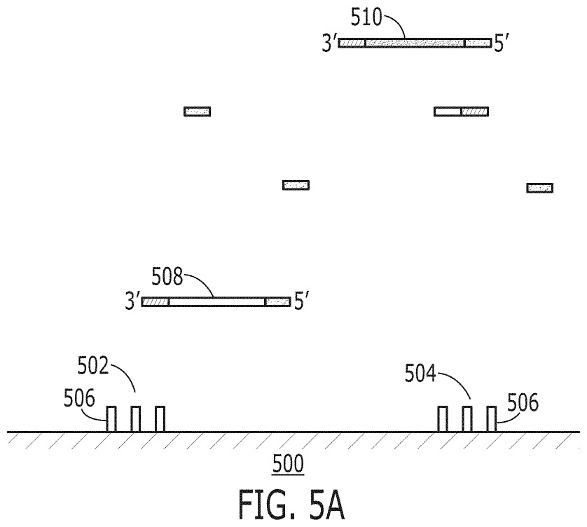

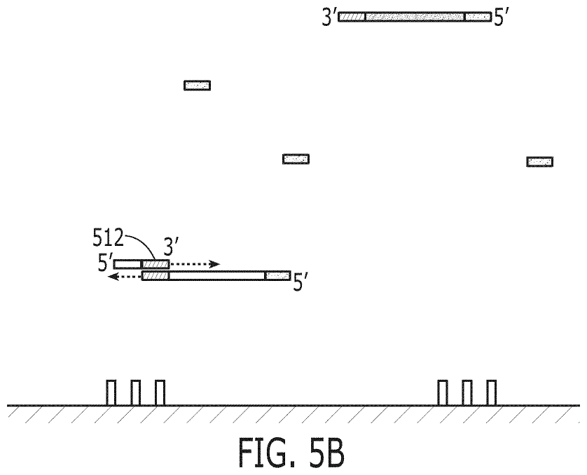

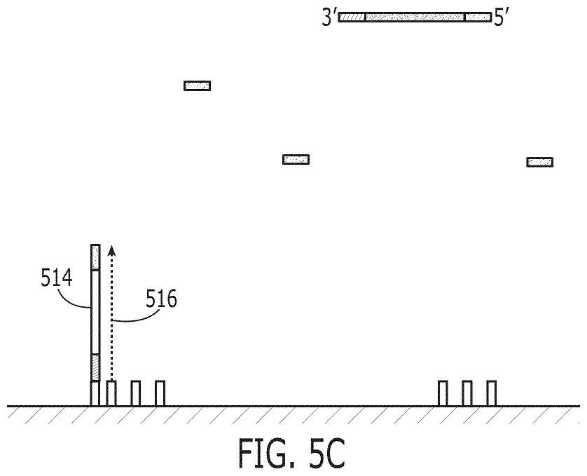

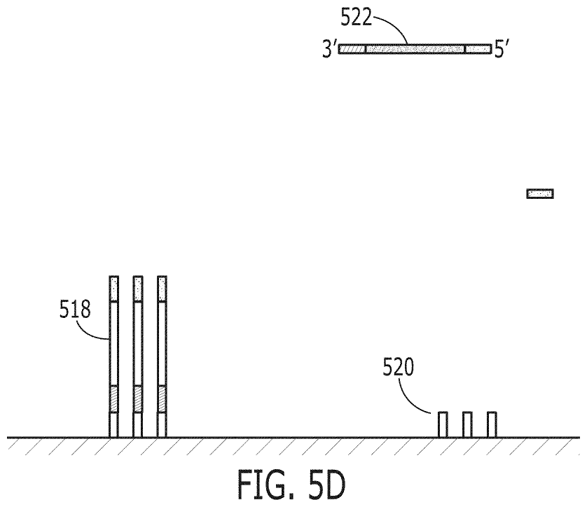

With reference to FIG. 5A, the method can be performed on a surface 500 such as a glass, plastic, silicon wafer, or any other suitable surface. The surface can have discrete regions 502, 504, each region having a plurality of the first primer 506 attached in the region of the surface. For example, each region may be separated by a sufficient gap region (having a lack of the first primer). In some instances, a minimum distance between any first primer in a first region and any first primer in a second region may be on the order of 10⁻¹, 10⁻², 10⁻³, 10⁻⁴, 10⁻⁵, 10⁻⁶, 10⁻⁷, 10⁻⁸, 10⁻⁹ m or less. A library of template nucleic acid molecules 508, 510 can be in fluidic contact with a plurality of the separated regions. That is, the methods described herein do not need to be performed in a plurality of emulsion droplets, though they may be.

The principal mechanisms on an open surface, with all of the components having fluidic access to a plurality of clusters of the first primer, can be similar to when performed in an emulsion. With reference to FIG. 5B, a second primer 512 can extend the first nucleic acid template to generate an extension product. In FIG. 5C, the extension product 514 can hybridize to one of the copies of the first primer on a first cluster on the open surface, which can be extended 516. The second primer 512 can be provided in limiting or low concentrations. Subsequent to the slow process of creating the extension product (e.g., due to the limiting or low concentrations of the necessary reaction reagent, the second primer), amplification on the surface can be faster such that substantially all of the copies of the first primer at a cluster location can be consumed (and be clonal) before another extension product derived from a second template can anneal at the same cluster location. With reference to FIG. 5D, a clonal cluster corresponding to a first nucleic acid template 518 can be created. Other cluster locations 520 can be available for clonal amplification of another nucleic acid template(s).

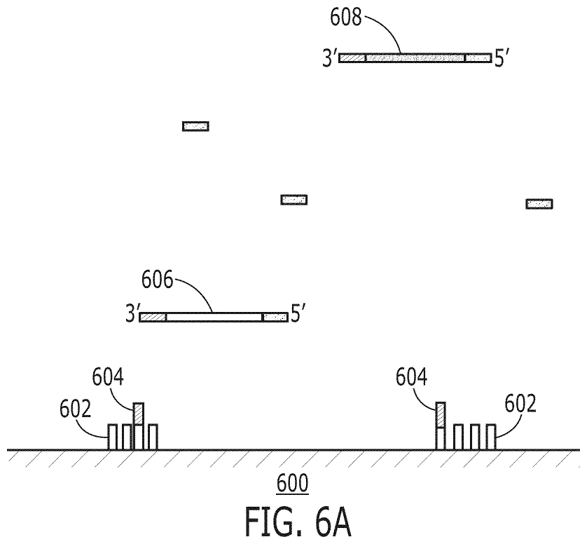

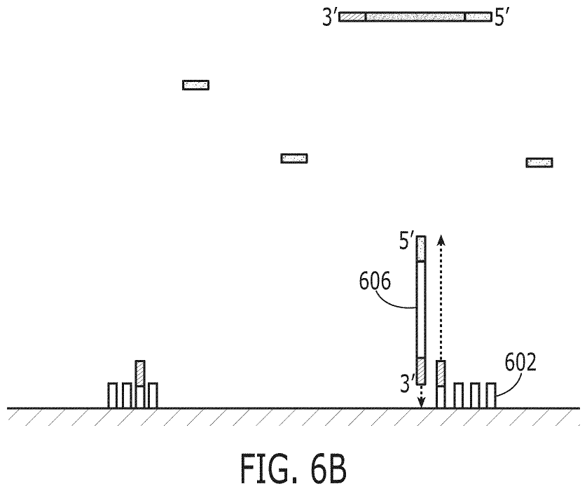

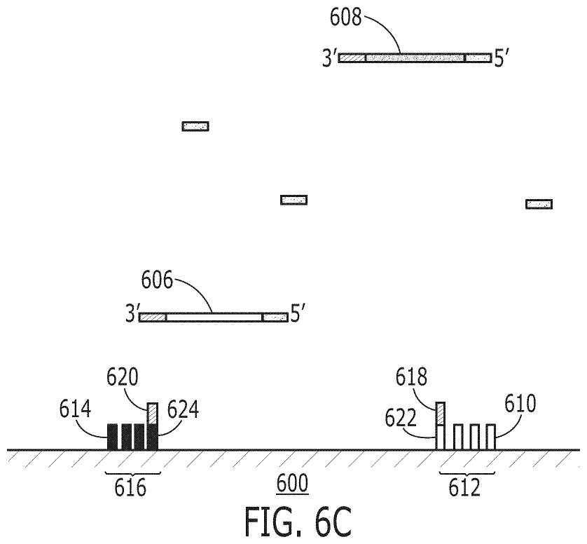

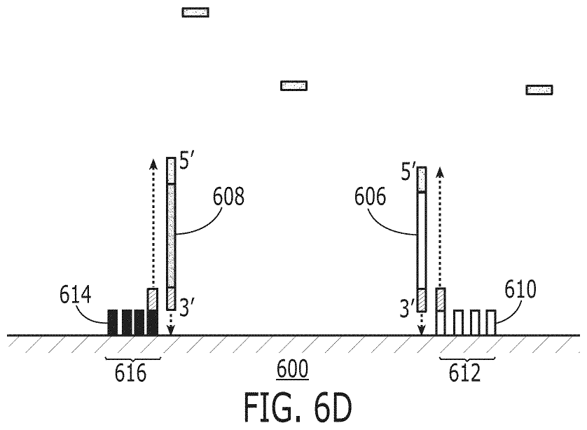

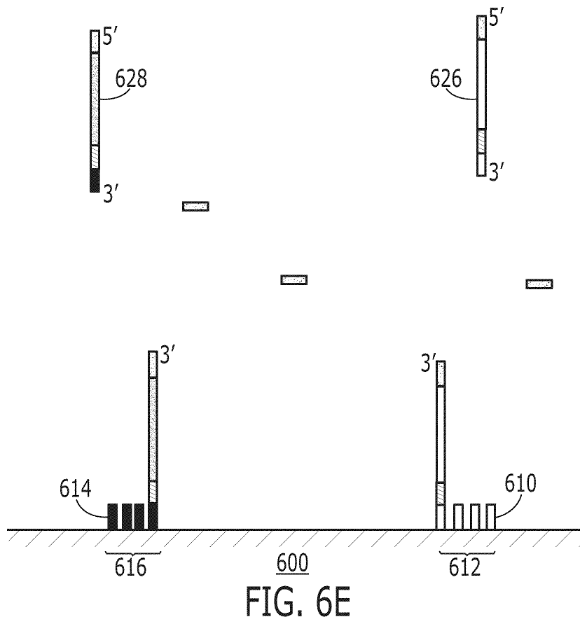

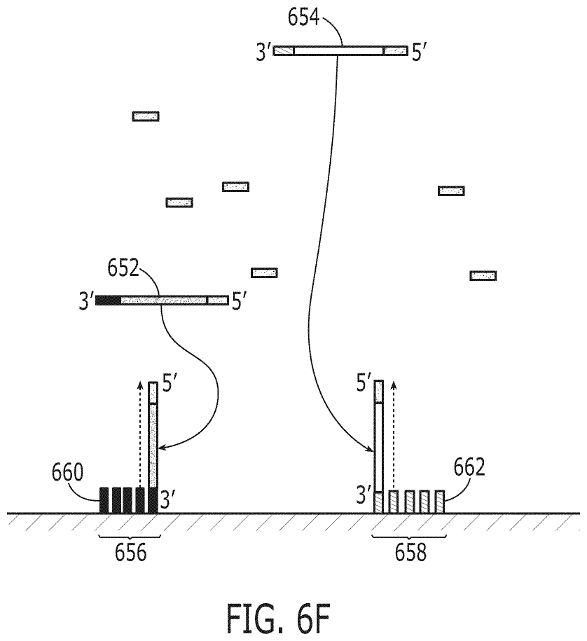

In some cases, the second primer is also attached to the surface (alternatively to or in addition to being present in solution). The concentration of the second primer can be limiting, e.g., low, relative to the number of the first primer attached in a cluster (or on a bead). An advantage of these embodiments can be that the initial processes of the method can be further slowed down in comparison with the later amplification processes that rapidly consume the local copies of the first primer. With reference to FIG. 6A, the surface 600 can have a plurality of clusters. The clusters can form an array (e.g., for DNA sequencing by imaging of distinct clusters). The clusters can have several copies of the first primer 602 and fewer copies of the second primer 604. In some cases, a ratio of the concentration of the second primer to the concentration of the first primer is on the order of about 10⁻¹, 10⁻², 10⁻³, 10⁻⁴, 10⁻⁵, 10⁻⁶, 10⁻⁷, 10⁻⁸, 10⁻⁹ or less. The clusters can be in fluidic contact with a nucleic acid library comprising a first template 606 and a second template 608. Continuing to FIG. 6B, the first nucleic acid template 606 can be extended with the second primer to create an extension product which can subsequently be amplified with the first primer 602 to create a clonal cluster.

In some embodiments, the respective sequences of the first primer are different at different cluster locations of the surface (or on different beads). With reference to FIG. 6C, the plurality of first primers 610 at a first cluster location 612 (or on a first bead) have a different sequence than a plurality of first primers 614 at a second cluster location 616 (or on a second bead). The second primers can also be different at different cluster or bead locations. In some cases, the second primers have a common first portion and different second portions. As shown in FIG. 6C, the first portion 618 of a first second primer located at a first cluster location 612 (or on a first bead) is the same as the first portion 620 of a second primer located at a second cluster location 616 (or on a second bead). However, the second portions of the respective second primers can be different. In some instances, the second portion 622 of the first second primer can be the same as the first primer 610. In some instances, the second portion 624 of the second primer can be the same as the second first primer 614.

Having the first primers be different at different cluster locations (or on different beads) can result in a template that is initially extended at a cluster location developing an additional affinity for that cluster location (with no additional affinity for other cluster locations). The extension region from a given cluster location provides additional base pairs of homology and increased affinity to the given cluster location compared with the affinity of the hybridization between the non-extended template and the second primer. The annealing reaction, the extension reaction, and/or the incubation of the emulsion can be performed at conditions (e.g., temperature) that are of sufficient stringency such that, without extension, the annealing and/or extension are rare and/or slow events.

Referring to FIG. 6D, a first template 606 can be extended at a first cluster location (or bead) 612 to yield a first extension product that comprises a region complementary to the first primer 610. A second template 608 can be extended at a second cluster location (or bead) 616 to yield a second extension product that comprises a region complementary to the second first primer 614. With reference to FIG. 6E, the first extension product 626 has a region of homology with, and can anneal to, and can provide a template for extension of, a first primer at the first cluster location 612 but not the second cluster location 616, i.e., because the first template was originally extended at the first cluster location. Similarly, the second extension product 628 has a region of homology with, and can anneal to, and can provide a template for extension of, a first primer at the second cluster location 616 but not the second cluster location 612, i.e., because the first template was originally extended at the second cluster location. Migration of extension products between cluster locations is not favored, especially if a temperature is used that is greater than or similar to an annealing temperature between the second primer and the nucleic acid template. Any solution described herein may refer to a bulk solution or an environment within a droplet (e.g., comprising a surface, e.g., a surface of a bead or other support).

In other instances, systems, compositions, and methods of the multiple unique supports, comprising unique primer species, described herein, may be implemented as clusters on an open surface. Referring to FIG. 6F, a first cluster 656 has a plurality of a first primer species 660 and a second cluster 658 has a plurality of a second primer species 662 different from the first primer species. The first primer species 660 can be configured to hybridize to a first adapter of a first template nucleic acid molecule 652, but not to a second adapter of a second template nucleic acid molecule 654. Similarly, the second primer species 662 may be configured to hybridize to the second adapter of the second template nucleic acid molecule 654, but not to the first adapter of the first template nucleic acid molecule 652. The first cluster 656 and the second cluster 658 may be in fluidic contact with a common solution comprising the mixture of template populations (e.g., comprising the first template nucleic acid molecule 652 and the second template nucleic acid molecule 654). Because of the different available primer species-adapter combinations, amplification using the clusters is likely to generate substantially monoclonal amplified populations at each cluster.

The present disclosure can involve an initial slow or rare attachment of a template to a surface followed by a rapid amplification of the surface-attached template (or derivative thereof) to use up the surface primers, providing a clonally amplified template (or derivative thereof). As described above, this can be accomplished by using an extension of the template to allow for attachment. Furthermore, another slow or rare step prior to amplification can be added to the methods described herein. The further slow step can also involve extension of the template, this time at an end distal from the end of the template that attaches to the surface.

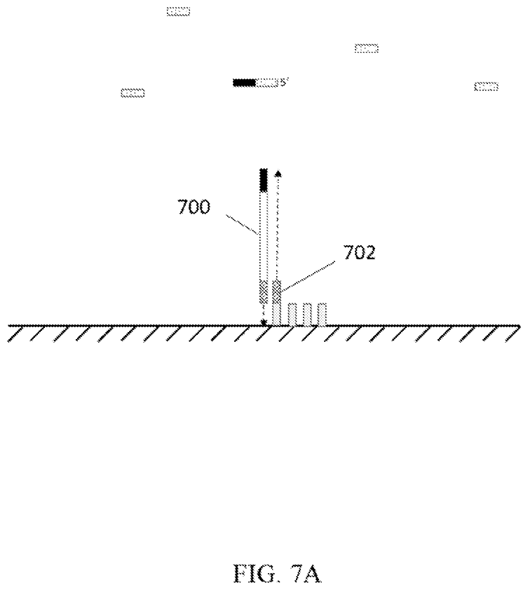

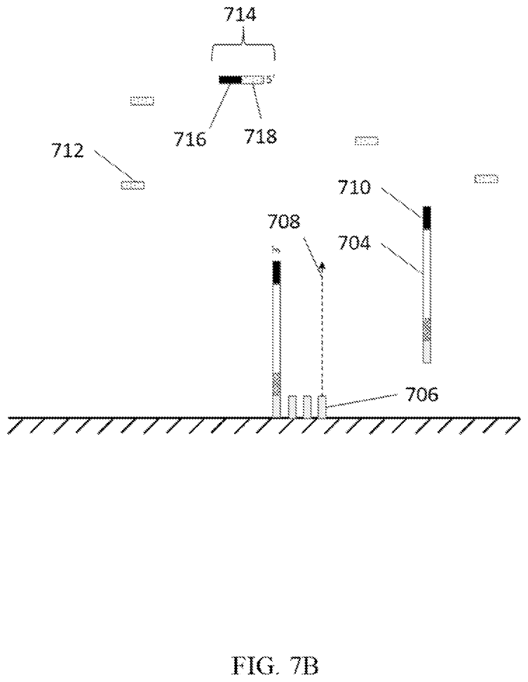

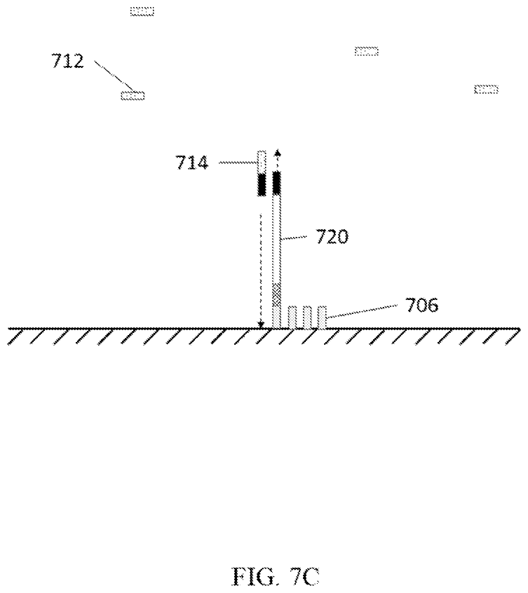

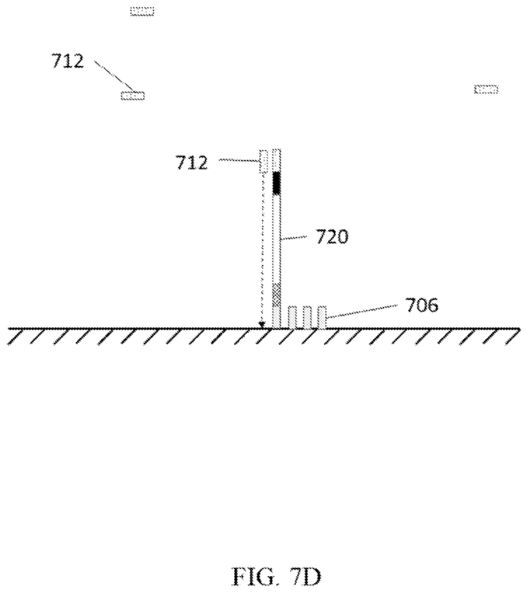

Referring to FIG. 7A, the systems, methods, and compositions can include a template 700 that anneals to a second primer 702 (immobilized on a surface) and extends to create an extension product. The second primer can be extended using the template nucleic acid molecule as a template, thereby creating the first copy of the eventual colony of nucleic acid molecules to be sequenced. Continuing with FIG. 7B, the extension product 704 can diffuse away from the second primer, hybridize with a copy of the first primer 706 attached to the surface, and serve as a template for extension of the first primer 708. However, this extension is linear rather than exponential, i.e., only one copy of the first (surface) primers are extended in each cycle. This is because the distal end 710 (an opposite end from the end that couples to a surface-immobilized primer) of the template and/or extension product 704 is not initially complementary with the third primer 712 in solution. The system can further include a fourth primer 714 having a first portion 716 and a second portion 718. The first portion can anneal to the nucleic acid template and the second portion can be capable of extending the nucleic acid template such that the extension product can hybridize with the third primer.

Continuing with FIG. 7C, the fourth primer 714 can further extend the previously extended copy (from either a first or second primer) immobilized to the surface 720. The fourth primer can also extend the template nucleic acid (or products thereof) in solution in some cases, e.g., when the template nucleic acid molecule is double stranded. In some cases, the template nucleic acid molecule is single stranded, and the fourth primer does not hybridize with the template nucleic acid molecule until it has been first extended with the first or second primer.

Following this extension, continuing with FIG. 7D, the third primer 712 is now able to be extended to create additional polynucleotides capable of serving as templates for the extension of additional copies of the first primer 706. The initial extensions of the original template at each end (using the second and fourth primers) can be relatively slow and rare events compared to exponential amplification (using the first and third primers). In some cases, both extensions need to be completed before exponential amplification can fill up the colony location defined by a cluster of the first primer attached to the surface.

In another aspect, provided herein is a method for clonally amplifying a nucleic acid sample. The method can include forming an emulsion having a plurality of partitions. A partition of the plurality of partitions can comprise a template nucleic acid, a bead having multiple copies of a first primer attached to the bead, and a reagent mixture capable of performing an attachment reaction that allows the template nucleic acid or a derivative thereof to attach to the bead and an amplification reaction that uses the multiple copies of the first primer. The method can further include incubating the emulsion, thereby performing the attachment reaction to attach the template nucleic acid or a derivative thereof to the bead and performing the amplification reaction to amplify the template nucleic acid or a derivative thereof that was attached to the bead.

In some cases, a first period of time (which duration is described below) is greater than a second period of time (which duration is described below). The first period of time can begin when the emulsion begins incubation and conclude when the template nucleic acid or derivative thereof attaches to the bead. In some cases, the second period of time can begin when the template nucleic acid or derivative thereof attaches to the bead and concludes when amplification reaction concludes. In some cases, for the purpose of defining the second period of time, an amplification reaction can be deemed concluded when the first primers on a bead or a cluster of the first primers of a cluster location on a surface is completely extended. In some cases, for the purpose of defining the second period of time, an amplification reaction can be deemed concluded when at least 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more first primers on a bead or cluster of the first primers of a cluster location on a surface has been extended.

The first period of time can be greater than the second period of time by any suitable factor. In some embodiments, the first period of time is about 5, about 10, about 20, about 50, or about 100 times greater than the second period of time. In some embodiments, the first period of time is at least about 5, at least about 10, at least about 20, at least about 50, or at least about 100 times greater than the second period of time.

### Extended Unique Primers

Provided herein are methods for generating and/or providing a support comprising an extended primer (e.g., second primer), as described elsewhere herein, e.g., for using extended supports on beads. Any of the supports described herein may be subsequently partitioned, such as during an ePCR operation. A support comprising at least one extended primer molecule (e.g., second primer) and/or at least one template nucleic acid molecule may generally be referred to herein as an extended support. For example, an extended support comprises the surface 600 with reference to FIG. 6A, which surface comprises a cluster of primers or a plurality of such clusters, and the cluster comprises a first number of first primers (e.g., 602) and a second number of second primers (e.g., 604). In some cases, the second number can be lower than the first number. For example, a ratio of the concentration of the second primer to the concentration of the first primer on the surface is on the order of about 10⁻¹, 10⁻², 10⁻³, 10⁻⁴, 10⁻⁵, 10⁻⁶, 10⁻⁷, 10⁻⁸, 10⁻⁹ or less. In an example operation, as described herein, a nucleic acid template couples (e.g., anneals) to the second primer attached to the surface and is subjected to a nucleic acid extension reaction to create an extension product, which extension product, or derivative thereof, can subsequently be amplified with the first primer attached to the surface. In some cases, the nucleic acid template may not be capable of annealing to the first primer. In another example, an extended support comprises a surface, the surface comprising a cluster of primers or a plurality of primers, and a template nucleic acid molecule is coupled to a primer of the cluster.

Provided herein are methods for isolating an extended support from a mixture of un-extended support(s) and extended support(s). In some instances, the support can be a mobile support (e.g., beads, particles, etc.) that are capable of being transported from a first location to a second location, individually or collectively with other supports. The support may be any support described elsewhere herein. A composition, mixture, or solution of isolated extended supports may be particularly beneficial for downstream operations, such as subsequent partitioning into droplets, as described elsewhere herein, where occupancy of such droplets generally follows the Poisson distribution which leads to the generation of a majority of droplets that are either unoccupied or singularly occupied in order to ensure generation of effective concentrations of singularly occupied droplets. Advantageously, if only extended supports are partitioned, the population of droplets occupied by supports will not be diluted by droplets containing un-extended supports which are more inefficient, if not unusable, for downstream operations (e.g., clonal amplification of libraries) than extended supports. Where an extended support comprises a template nucleic acid molecule coupled thereto, a double Poisson distribution (for each of the support occupancy and template occupancy in droplets) may be reduced to a single Poisson distribution (for a single template-support assembly occupancy in droplets). Extended supports may also beneficially allow for overloading of droplets (e.g., more than one in a droplet), as described elsewhere herein. Partitions other than droplets, such as wells or other containers, may be used. Extended supports may also benefit for use in bulk solution or open reaction space, as described elsewhere herein.

### II. Extended Supports

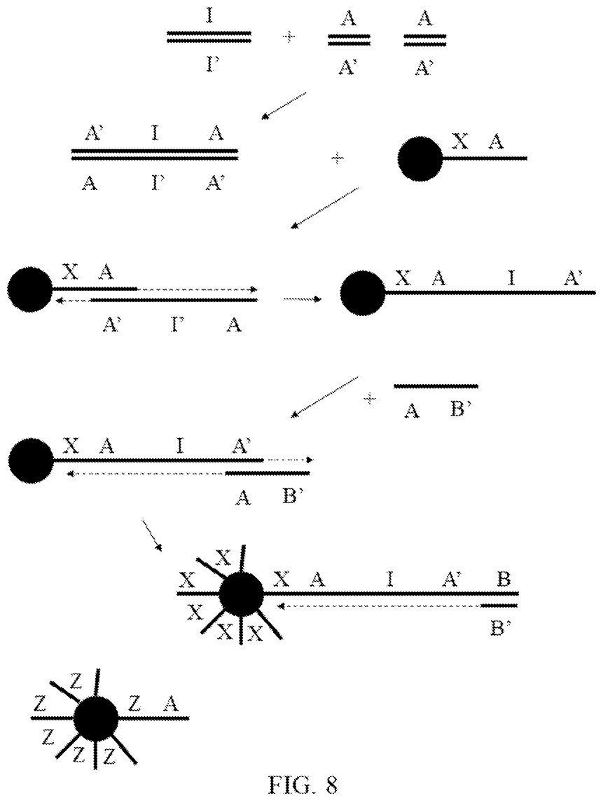

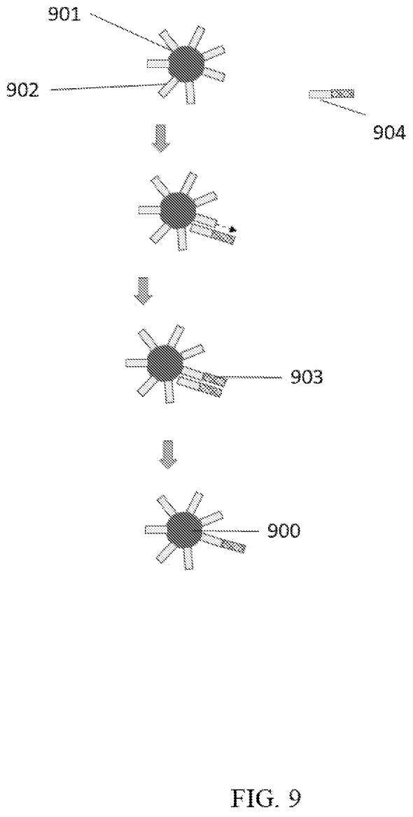

FIG. 9 illustrates an example method for generating and/or providing an extended support 900, wherein the extended support 900 comprises a plurality of primers attached thereto a surface of the support (e.g., a bead). The plurality of primers may comprise one or more of the first primer 902 and one or more of the second primer 903. In some instances, it may be of particular interest to generate an extended support 900 comprising a cluster which comprises a relatively fewer number of the second primer 903 compared to that of the first primer 902. In some cases, the extended support has one copy of the second primer 903 attached thereto. In other cases, the extended support has more than one copy of the second primer 903, such as a few copies or several copies of the second primer 903 (not illustrated). Alternatively or in addition, there may be a higher number or concentration of the first primer 902 than that of the second primer 903 attached to the surface of the support. In an example, when the extended support is used for sample preparation (e.g., for amplification of nucleic acid templates), as described herein, a nucleic acid template may be extended with the second primer, on rare occasions and therefore in a rate-limiting operation, to create an extension product which can subsequently be amplified with the first primer, which amplification can occur at significantly faster rates than the initial extension product generation reaction as there are more copies of the first primer than the second primer provided on the support. In some cases, the amplification reaction may exhaust (e.g., by coupling thereto) the copies of the first primer on the extended support before another nucleic acid template can be extended with another second primer (if any) in the reaction mixture, thereby facilitating a monoclonal population on the support (or within a cluster on the support).

A starting support 901 (or un-extended support) may comprise a first primer 902. The starting support may comprise a plurality of the first primer, such as a cluster of the first primer. The first primer can be attached to an extension primer 904 for example via hybridization of complementary sequences, and subsequently extended to generate an extended primer, the second primer 903, that is immobilized to the support. The attachment reaction (e.g., hybridization) may be performed in a solution, such as in bulk solution comprising a plurality of un-extended supports and/or in emulsion comprising a partition comprising an un-extended support. The attachment process (such as hybridization) may comprise a single cycle extension process. After the second primer is generated, a washing and/or melting operation may be performed to disassociate the extension primer to generate the extended support 900.

In some instances, the respective concentrations of the un-extended support (e.g., 901) and the extension primer (e.g., 904) in a reaction mixture may be modulated to facilitate generation of an extended support comprising a minimal number (e.g., one, a few, several, etc.) of the second primer. For example, the extended support may comprise at least about 50%, 60%, 70%, 80%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.9999% or more of the first primer (out of a total of the first primer and the second primer population).

For example, the reaction mixture may contain a fewer number or less concentration of the extension primer relative to the number or concentration of the first primer present (e.g., via attachment to the un-extended support). In some instances, the ratio of a concentration of extension primers to a concentration of un-extended supports in a solution is at most about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:100, 1:500, 1:1000, 1:5000, 1:10000, or less. Alternatively or in addition, the ratio of a concentration of extension primers to a concentration of un-extended supports in a solution is at least about 1:50, 1:40, 1:30, 1:20, 1:29, 1:18, 1:17, 1:16, 1:14, 1:13, 1:12, 1:11, 1:10, or greater. In some instances, the percentage of a concentration of extension primers to a concentration of un-extended supports in a solution is at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less. Alternatively or in addition, the percentage of a concentration of extension primers to a concentration of un-extended supports is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or greater. A resulting mixture may comprise a mixture of extended support(s) and un-extended support(s) that remain un-extended.

Provided herein are methods of isolating an extended support from a mixture of un-extended support(s) and extended support(s).

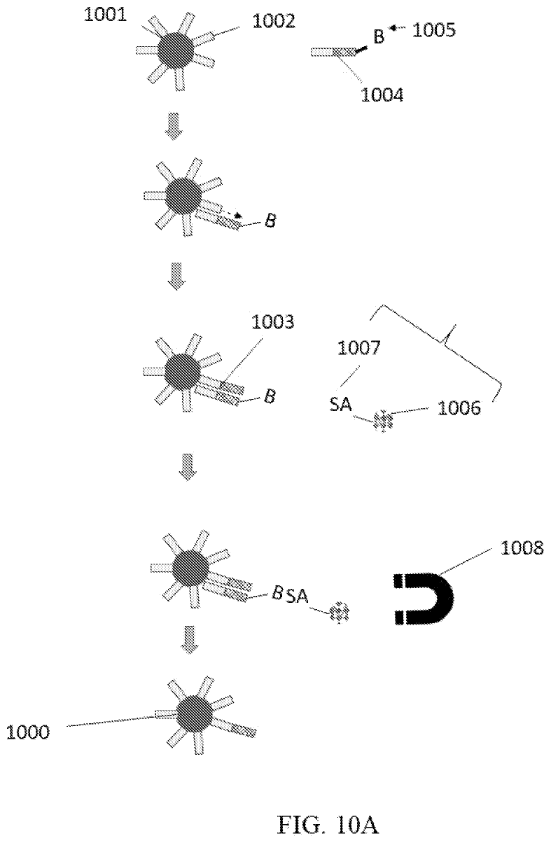

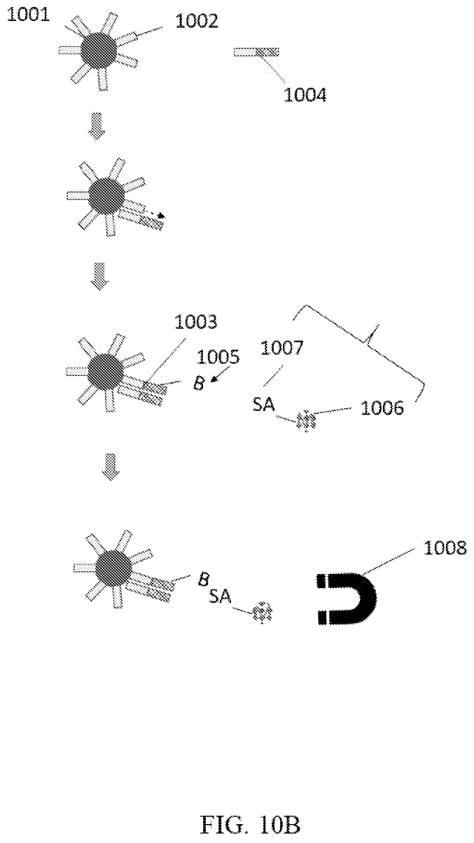

FIG. 10A illustrates an example method for isolating an extended support. A starting support 1001 (or un-extended support) may comprise a first primer 1002. The starting support may comprise a plurality of the first primer, such as a cluster of the first primer. The starting support may be brought in contact with an extension group 1004. The first primer can be attached to the extension group. The extension group may comprise a primer molecule comprising a capture entity 1005. In some instances, the capture entity may comprise biotin (B), such that the primer molecule is biotinylated. In some instances, the capture entity may comprise a capture sequence (e.g., nucleic acid sequence). In some instances, a sequence of the primer molecule may function as a capture sequence. In other instances, the capture entity may comprise another nucleic acid molecule comprising a capture sequence. In some instances, the capture entity may comprise a magnetic particle capable of capture by application of a magnetic field. In some instances, the capture entity may comprise a charged particle capable of capture by application of an electric field. In some instances, the capture entity may comprise one or more other mechanisms configured for, or capable of, capture by a capturing entity.

The first primer 1002 may attach to the extension group 1004, for example via hybridization of complementary sequences (e.g., between a sequence of the first primer 1002 and a sequence of the primer molecule), and subsequently extended to generate an extended primer, the second primer 1003, that is immobilized to the support. The attachment reaction (e.g., hybridization) may be performed in a solution, such as in bulk solution comprising a plurality of un-extended supports and/or in emulsion comprising a partition comprising an un-extended support. The attachment process (such as hybridization) may comprise a single cycle extension process. After the second primer is generated, the extension group 1004 may remain associated with the first primer 1002 and immobilized to the support.

Alternatively, referring to FIG. 10B, a starting support 1001 (or un-extended support) may comprise a first primer 1002. The starting support may comprise a plurality of the first primer, such as a cluster of the first primer. The starting support may be brought in contact with an extension group 1004. The first primer can be attached to the extension group. The extension group in this example lacks a capture entity 1005. The first primer 1002 may attach to the extension group 1004, for example via hybridization of complementary sequences (e.g., between a sequence of the first primer 1002 and a sequence of the primer molecule), and subsequently extended to generate an extended primer, the second primer 1003, that is immobilized to the support. For the extension reaction, reagents comprising the capture entity (e.g., a nucleotide comprising the capture entity, such as a biotin) may be used resulting in the second primer 1003 comprising the capture entity 1005. In some instances, the capture entity may be biotin (B), such that biotin labeled nucleotides are used for the extension reaction. A single labeled base may be employed, such as labeled adenine, labeled thymine, labeled guanine, or labeled cytosine, or analogs thereof. The labeled nucleotide may be selected based on the sequence of the extension group 1004. In an example, only a single labeled nucleotide is added. This can be achieved by selecting a sequence for the extension group 1004 that comprises only one residue of a particular base. Alternatively, the extension can be performed in two operations. In the first operation, only the first nucleotide is added, and this nucleotide is labeled with the capture entity 1005. A second extension reaction is performed with all the bases, wherein no labeled bases are used. This results in a second primer 1003 comprising only one capture entity 1005. Alternatively, the stepwise single labeled nucleotide addition can be performed at any other position of the extension (e.g., second position, third position, fourth position, etc.). In some instances, the capture entity may comprise a capture sequence (e.g., nucleic acid sequence). In some instances, the complement of the extension group 1004 is the capture sequence, such that the second primer 1003 comprises the capture sequence.

Referring back to FIG. 10A, the support comprising the extension group 1004 attached thereto may be brought in contact with, or otherwise subjected to capture by, a capturing group 1020. In some instances, the capturing group may comprise a capturing entity 1007 configured to capture the capture entity 1005. For example, the capturing entity may be configured to target the capture entity. In some instances, the capturing entity may comprise streptavidin (SA) when the capture moiety comprises biotin. In some instances, the capturing entity may comprise a complementary capture sequence when the capture entity comprises a capture sequence (e.g., a capture oligonucleotide that is complementary to the complementary capture sequence). In some instances, the capturing entity may comprise an apparatus, system, or device configured to apply a magnetic field when the capture entity comprises a magnetic particle. In some instances, the capturing entity may comprise an apparatus, system, or device configured to apply an electrical field when the capture entity comprises a charged particle. In some instances, the capturing entity may comprise one or more other mechanisms configured to capture the capture entity. In some instances, the capturing group may comprise a secondary capture entity 1006, for example, for subsequent capture by a secondary capturing entity 1008. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

When the support comprising the extension group 1004 attached thereto is brought in contact with, or otherwise subject to capture by, the capturing group 1020, the capturing entity 1007 of the capturing group may bind, couple, hybridize, or otherwise associate with the capture entity 1005 immobilized to the support. The association between the capture entity and the capturing entity may comprise formation of a non-covalent bond. The association may comprise formation of a covalent bond. The association may comprise formation of a releasable bond, for example, upon application of a stimulus. In some instances, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities. The capture entity/capturing entity pair may be any combination. The pair may include, but is not limited to, biotin/streptavidin, azide/cyclooctyne, and thiol/maleimide. It will be appreciated by a skilled artisan that either molecule of the pair may be used as either the capture entity or the capturing entity, the capture entity capable of linking to a nucleotide. Chemically modified bases comprising biotin, an azide, cyclooctyne, tetrazole, and a thiol, and many others are suitable as capture entities.

A plurality of un-extended supports and a plurality of extension groups may be subject to the operations described herein in a bulk solution. In some instances, the respective concentrations of the un-extended support and the extension group in a reaction mixture may be modulated to facilitate generation of an extended support comprising a minimal number (e.g., one, a few, several, etc.) of the second primer. For example, the reaction mixture may contain a fewer number or less concentration of the extension primer (e.g., primer molecule) relative to the number or concentration of the first primer present (e.g., via attachment to the un-extended support). In some instances, the ratio of a concentration of extension groups to a concentration of un-extended supports in a solution is at most about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:100, 1:500, 1:1000, 1:5000, 1:10000 or less. Alternatively or in addition, the ratio of a concentration of extension groups to a concentration of un-extended supports in a solution is at least about 1:50, 1:40, 1:30, 1:20, 1:29, 1:18, 1:17, 1:16, 1:14, 1:13, 1:12, 1:11, 1:10, or greater. In some instances, the percentage of a concentration of extension groups to a concentration of un-extended supports in a solution is at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less. Alternatively or in addition, the percentage of a concentration of extension groups to a concentration of un-extended supports is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or greater. A resulting mixture may comprise a mixture of extended support(s) and un-extended support(s) that remain un-extended.

In some instances, the capturing group may isolate the extended support from a mixture of extended support(s) (each comprising the extension group attached thereto) and un-extended support(s) (not attached to an extension group) by targeting the extension group attached thereto. In some instances, the capturing group may isolate multiple extended supports from a mixture of extended support(s) (each comprising the extension group attached thereto) and un-extended support(s) (not attached to an extension group) by targeting the respective extension groups attached thereto. In some instances, a plurality of capturing groups may be used to isolate the extended support from a mixture of extended support(s) (each comprising the extension group attached thereto) and un-extended support(s) (not attached to an extension group) by targeting the extension group attached thereto.

Once isolated, a washing and/or melting operation may be performed to disassociate the extension group from the support to provide the extended support 1000.

In some instances, the capturing group 1020 may associate with the extended support without isolation of the extended support from the mixture. In some instances, where the capturing group further comprises a secondary capture entity 1006, the support may remain associated with the secondary capture entity in the mixture. The support may be brought into contact with, or otherwise subject to capture by, a secondary capturing entity 1008. The secondary capturing entity may bind, couple, hybridize, or otherwise associate with the secondary capture entity of the capturing group. The association between the secondary capture entity and the secondary capturing entity may comprise formation of a non-covalent bond. The association may comprise formation of a covalent bond. The association may comprise formation of a releasable bond, for example, upon application of a stimulus. In some instances, the association may not form any bond. For example, the association may increase a physical proximity (e.g., decrease physical distance) of the secondary capturing entity and secondary capture entity. In some instances, a single secondary capture entity may be capable of associating with a single secondary capturing entity. Alternatively, a single secondary capture entity may be capable of associating with multiple secondary capturing entities. Alternatively or in addition, a single secondary capturing entity may be capable of associating with multiple secondary capture entities. In some instances, the secondary capturing group may isolate the extended support from a mixture of extended support(s) (each comprising the capture group attached thereto) and un-extended support(s) (not attached to a capture group) by targeting the capture group attached thereto. In some instances, the secondary capturing group may isolate multiple extended supports from a mixture of extended support(s) (each comprising the capture group attached thereto) and un-extended support(s) (not attached to a capture group) by targeting the respective capture groups attached thereto. In some instances, a plurality of secondary capturing groups may be used to isolate the extended support from a mixture of extended support(s) (each comprising the capture group attached thereto) and un-extended support(s) (not attached to a capture group) by targeting the capture group attached thereto.

Once isolated, a washing and/or melting operation may be performed to disassociate the extension group and the capture group (and in some cases also the secondary capturing entity) from the support to provide the extended support 1000.

In some instances, the secondary capturing entity 1008 may associate with the extended support without isolation of the extended support from the mixture. In some cases, the secondary capturing entity may comprise a third capture entity configured for subsequent capture by a third capturing entity (not illustrated). It will be appreciated that any degree of capturing entity may comprise another capture group that may be captured by a next degree of capturing entity, for isolation from the mixture and/or association by the next degree of capturing entity. Once isolated, a washing and/or melting operation may be performed to disassociate the extension group (and any number of capture entities and/or capturing entities) from the support to provide the extended support 1000.

In an example operation, a plurality of supports each comprising a plurality of first primers is brought in contact with a plurality of extension groups each comprising a biotinylated primer molecule. In some instances, the primer molecule attaches to the first primer and subject to nucleic acid extension to generate the second primer immobilized to the support. The support remains associated with the biotinylated primer molecule and is brought in contact with a capture group comprising a streptavidin coupled to a magnetic bead. The streptavidin binds to the biotin, thereby associating the magnetic bead with the support. In some instances, a support does not come into contact with an extension group and is not associated with the magnetic bead. For example, a mixture may comprise extended support(s) associated with magnetic bead(s) and un-extended support(s) unassociated with a magnetic bead. A magnet is used, or other magnetic field is applied, to target the magnetic bead(s) and isolate the extended support(s) associated with the magnetic bead(s) from the mixture. A resulting isolated composition comprises only extended support(s) or a majority of extended support(s). It will be appreciated that there may be some contamination in the isolated composition. A washing operation is performed to disassociate the extension group(s) and/or the capture group(s) from the extended support(s).

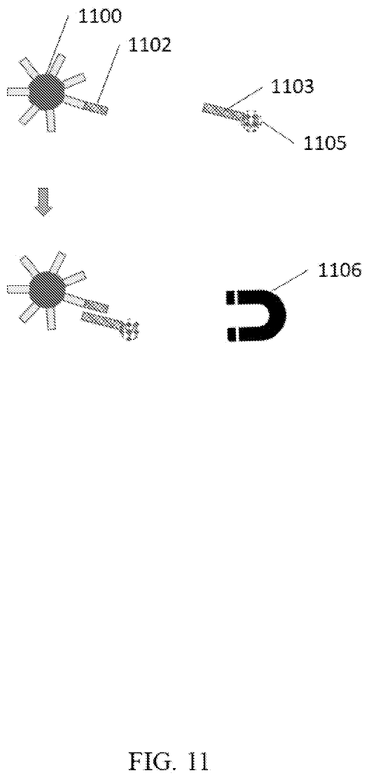

FIG. 11 illustrates another example method for isolating an extended support. An extended support 1100 comprising a second primer 1102 may be provided, such as according to methods described with respect to FIG. 9. A capture group may be provided, comprising a capture entity 1105 (e.g., magnetic bead) and a nucleic acid sequence 1103 attached thereto. The nucleic acid sequence attached to the capture entity may comprise sequence homology with a sequence of the second primer attached to the extended support. The capture group may be associated with the extended support such as via hybridization of the nucleic acid sequence and the sequence of the second primer, thereby associating the capture entity with the extended support. The capture group-associated support can be brought into contact with, or otherwise subject to capture by, a capturing entity 1106 (e.g., magnet). The capture group, and/or the capture entity, may be capable of disassociating from the extended support after association. In some instances, the capture group may be reused. In some instances, the capture entity may be reused. In some instances, a nucleic acid molecule comprising the nucleic acid sequence 1103 may be reused. Reusing the different reagents may be a cost-effective approach for isolation of the extended supports. The capture entity and capturing entity may comprise any one or more of the capturing mechanisms described elsewhere herein (e.g., biotin and streptavidin, complementary capture sequences, magnetic particle and magnetic field, charged particle and electric field, etc.). For example, the capture entity may comprise a particle having magnetic properties and the capturing entity may be configured to apply a magnetic field. For example, the capture entity may comprise a charged particle carrying an electrical charge and the capturing entity may be configured to apply an electric field. For example, the capture entity may comprise a nucleic acid capture sequence and the capturing entity may comprise a complementary nucleic acid capture sequence. It will be understood by a skilled artisan that the nucleic acid sequence 1103 may be used to attach a capture entity 1105 directly to the second primer 1102. As was described in FIG. 10B, an extension reaction may be used to add a capture sequence or a modified nucleotide comprising the capture entity 1105 directly to a primer on the support. In FIG. 10B the primer was a first primer, but it will be understood that the primer can be a second primer 1102 as well.

Other methods may be used to separate the extended supports from a mixture solution. Such separation methods may comprise using one or more other sequences or moieties capable of binding the extended supports (e.g., 1100), thereby separating the extended supports from the rest of the support population. In some examples, such sequences or moieties may comprise a higher or significantly higher binding affinity for the extended supports compared to the rest of the reagents, materials, and/or moieties present in the solution. Therefore, such sequences or moieties (referred to herein as separation moieties) may be capable of binding to, associating with, and/or capturing the extended supports. Separating the extended supports from the rest of the solution may contribute to providing a more purified composition of the extended supports, which in some examples may be used as a reagent in an experiment, assay, or procedure, such as the methods and systems described elsewhere herein.

The extended supports may be generated, separated, manufactured, and/or prepared as a reagent. The extended supports may be included in a kit, such as an experimental kit or test. The extended supports may be used in experiments or other procedures. For example, a kit may comprise a composition comprising an extended support reagent solution having at least about 50%, 60%, 70%, 80%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or greater purity (concentration of extended supports to combined concentration of extended supports and un-extended supports).

The present disclosure provides methods of using the extended supports in experiments. For example, the extended supports may be provided with a library of sequences, such as a library of nucleic acid sequences to be analyzed in an experiment (such as a sequencing experiment, for example next generation sequencing, or any other type of sequencing). The library of sequences may comprise nucleic acid sequences, which may comprise one or more adapter sequences attached thereto. The library of sequences may comprise template nucleic acid molecules comprising template nucleic acid sequences. The template nucleic acid molecules may comprise one or more adapter sequences attached thereto. For example, a template nucleic acid molecule may comprise an adapter sequence flanking a first end. In another example, a template nucleic acid molecule may comprise the same or different adapter sequences flanking the two ends of a template sequence. Alternatively, the template nucleic acid molecules may not comprise adapter sequences (e.g., a template nucleic acid molecule may have only a template sequence).

In some examples, the extended supports may be mixed with the library of nucleic acid sequences and the mixture may be subject to conditions sufficient to initiate a nucleic acid extension reaction that can immobilize a template nucleic acid sequence (or complement thereof) to the support. In some instances, such reaction may be performed in partitions (e.g., droplets in an emulsion), as described elsewhere herein, wherein a partition comprises one or more extended supports and one or more template nucleic acid sequences. In other instances, such reaction may be performed in bulk solution. In some examples, the extended supports may be immobilized to a reaction space, and such reaction performed using a bulk solution that fluidically contacts the reaction space. In some examples, immobilization (e.g., hybridization) may be performed in solution (e.g., off-chip), and after immobilization in solution, the immobilized assemblies (combination of the extended support and the template nucleic acid molecule) may be encapsulated in partitions (such as partitions described herein) for subsequent operation. In some examples, partitions are droplets. In some examples, partitions are wells.

### III. Enrichment

Provided herein are methods for enriching amplified primers that are immobilized on the surface of a support (e.g., a bead). Amplified primers are those that are attached to a template nucleic acid molecule of a biological sample. The support comprising such amplified primer can itself be immobilized on a substrate surface using any of the conjugation strategies described herein. The present disclosure may generally refer to a “positive” support, e.g., one that comprises a template nucleic acid molecule attached to its surface pre-amplification or post-amplification, and a “negative” support, e.g., one that does not comprise a template nucleic acid molecule attached to its surface. A positive support can be immobilized on a substrate surface before amplification of such template nucleic acid molecule. In some cases, such positive support can be a unique support species such as a unique bead species. Thus, nucleic acid amplification can be performed while the support (e.g., bead) is attached to the substrate surface. Such amplification can comprise recombinase polymerase amplification (RPA). Examples of on-bead amplification are described with respect to FIGS. 3C-3G. Following amplification, the supports, which can comprise template nucleic acid molecules and amplicons thereof attached to their surfaces, can be analyzed (e.g., sequenced). In some cases, the supports are detached from the substrate surface and analyzed (e.g., sequenced). In some cases, the supports remain immobilized to the substrate surface when analyzed (e.g., sequenced), for example, after subjecting the reaction space to washing. Unique support species can be used to facilitate production of monoclonal supports during amplification. In other instances, a positive bead can be amplified before such support is immobilized on a substrate surface.

Further provided herein are methods for increasing the sensitivity in sequencing workflows that can utilize both uniform support species as well as unique support species as described herein. Such methods comprise enriching amplified surface primers (e.g., those that are located on the surface of a support such as a bead) that are attached to template nucleic acid molecules.

Methods for enrichment provided herein can comprise immobilizing the beads on a suitable substrate surface. Such attachment of beads to a surface can be achieved using various bioconjugation chemistries described herein, e.g., one that uses modified nucleotides. In one example, a solution comprising a plurality of template nucleic acid molecules can be brought into contact with a plurality of supports to generate a number of positive beads, where a positive bead comprises a template nucleic acid molecule annealed to a surface primer. In some instances, the beads can be provided in a large excess over the template nucleic acid molecules in the mixture, to result in a distribution of bead-hybridized products, with a large number of “empty” beads containing only unreacted primers, some number of beads with two or more template nucleic acid molecules captured, and a substantial amount of the desired beads annealed to a single template nucleic acid molecule. The positive bead may comprise modified nucleotide(s) which, for example, may be introduced to the template-surface primer complex via the template nucleic acid molecule (incorporated into or coupled to the template nucleic acid molecule) or via one or more extension reactions of the surface primer (which incorporates the modified nucleotide(s)). In a subsequent operation, the solution of the beads can be dispersed over a solid surface modified with a chemically reactive residue that will allow the specific capture of only those beads that carry the modified nucleotide(s). Thus, no negative beads or no significant amounts of such beads are likely to be captured as they do not contain modified nucleotides. In some cases, however, beads that can carry two or more different template nucleic acid molecules captured in the original hybridization step may still be captured; their number can depend on the bead to template ratio used in the first hybridization step and can be minimized by increasing that ratio. Next, the enrichments methods herein can comprise a wash step to remove any beads and other components that are not surface-bound. The result can be a surface coated with immobilized beads, all of which, or the majority of which, carry at least one template nucleic acid molecule. The substrate surface can then be subjected to an enzymatic amplification reaction, such as the recombinase polymerase amplification (RPA) described elsewhere herein, to enzymatically multiply the number of copies of the individual template nucleic acid molecules captured on the beads. The resulting amplified template nucleic acid molecules can then be subjected to a sequencing process.

In some cases, the plurality of template nucleic acid molecules can have sequence identity of, e.g., at least about 50%, 60%, 70%, 75%, 80%, 85%, 90%, 95%, 97%, or 99% sequence identity. Similarly, the first and second pluralities of single-stranded nucleic acid molecules can have sequence identity of, e.g., at least about 50%, 60%, 70%, 75%, 80%, 85%, 90%, 95%, 97%, or 99% sequence identity.

Further provided herein are systems and methods for enrichment which can selectively remove or degrade a set of nucleic acid molecules from a plurality of supports (e.g., beads), to subsequently isolate positive supports from negative supports. In the process, those positive supports may be processed to remove unreacted surface primer molecules, or ‘cleaned up,’ to reduce downstream noise and increase sequencing sensitivity.

A method for processing supports can comprise (a) providing a plurality of supports (e.g., beads), wherein the plurality of supports comprises a plurality of surface primer molecules coupled thereto. A plurality of template nucleic acid molecules (e.g., from or derived from a biological sample to be sequenced) can be immobilized to a support of the plurality of supports via a first set of surface primer molecules of the plurality of surface primer molecules. A second set of surface primer molecules of the plurality of surface primer molecules may not be coupled to the plurality of template nucleic acid molecules. The method can comprise (b) selectively removing or degrading the second set of surface primer molecules from at least a subset of the plurality of supports to yield a plurality of processed supports, wherein the plurality of processed supports comprises, or retains, the plurality of template nucleic acid molecules.

The support may be a positive support post-amplification which comprises the plurality of template nucleic acid molecules. For example, the plurality of template nucleic acid molecules may be substantially monoclonal and/or comprise amplicons of an original template nucleic acid molecule, or derivative thereof. The plurality of template nucleic acid molecules can be immobilized to the support via the first set of surface primer molecules, where such immobilization can occur through, e.g., hybridization between the reverse complements of template nucleic acid molecules, or portions thereof, and the first set of surface primer molecules, followed by extension of the first set of surface primer molecules.

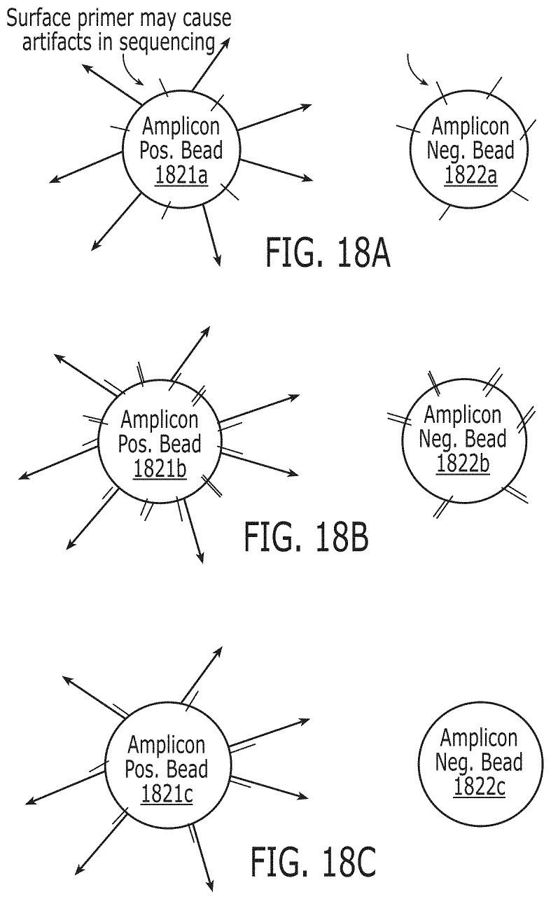

With reference to FIG. 18A, the plurality of template nucleic acid molecules can be single-stranded nucleic acid molecules. A plurality of supports, of which at least a subset of supports (e.g., 1821a) contains one or more template nucleic acid molecules immobilized thereto via at least the first set of surface primer molecules, can be contacted with a reaction mixture that comprises a plurality of single-stranded nucleic acid molecules that have at least partial sequence complementarity to the plurality of surface primer molecules (e.g., indicated by arrows in FIG. 18A). Another subset of supports (e.g., 1822a) may not be coupled to any template nucleic acid molecule. With reference to FIG. 18B, the plurality of single-stranded nucleic acid molecules can be used to generate a plurality of double-stranded nucleic acid molecules (e.g., blunt-ended, partially double stranded) coupled to at least a subset of the plurality of supports. The plurality of double-stranded nucleic acid molecules can comprise at least a subset of the plurality of single-stranded nucleic acid molecules coupled (e.g., hybridized) to a second set of unreacted and/or non-amplified surface primer molecules. Thus, the plurality of single-stranded nucleic acid molecules can be used to occupy all remaining free and unbound (single-stranded) surface primer molecules of the supports, which may have templates (e.g., 1821b) or may be without templates (e.g., 1822b). In some instances, an additional subset of the plurality of single-stranded nucleic acid molecules may be coupled to the amplified surface primer molecules, that is, the first set of surface primer molecules that have been extended with the template sequence (or complement thereof), such as to yield partially double-stranded nucleic acid molecules which have a 3′ overhang.

The resulting double-stranded molecules can comprise a plurality of partially double-stranded nucleic acid molecules which have a 5′ overhang and/or a 3′ overhang. In some instances, where there is a 3′ overhang, the overhang may be less than 4 bases in length. An example of a partially double-stranded nucleic acid molecule is an unreacted surface primer molecule which is hybridized to a single-stranded nucleic acid molecule having partial sequence complementarity such that there is a 5′ overhang (e.g., proximal to the support surface) and/or a 3′ overhang (e.g., distal to the support surface). Where there is a 3′ overhang, the overhang may be less than 4 bases in length. Another example of a partially double-stranded nucleic acid molecule is an amplified surface primer molecule which is hybridized to a single-stranded nucleic acid molecule having sequence complementarity such that there is a large 3′ overhang (e.g., equal to or greater than 4 bases in length), the 3′ overhang corresponding to the template sequence (or complement thereof and/or portion thereof). Another example of a partially double-stranded nucleic acid molecule is an amplified surface primer molecule which is hybridized to a single-stranded nucleic acid molecule having partial sequence complementarity such that there is a 5′ overhang and a large 3′ overhang (e.g., equal to or greater than 4 bases in length).

In some cases, the plurality of single-stranded nucleic acid molecules may have a protective moiety at a 3′ end of the second single-stranded nucleic acid molecule (e.g., proximal to the support surface when coupled to the surface primer molecules), such as a phosphorothioate moiety.

In some cases, the first set of surface primer molecules that are coupled to template nucleic acid molecules can be mutually exclusive to the second set of surface primer molecules that are hybridized to at least a subset of the single-stranded nucleic acid molecules to form the plurality of double-stranded nucleic acid molecules.

With reference to FIG. 18C, the double-stranded nucleic acid molecules (e.g., comprising unreacted surface primer molecules) can then selectively be removed or degraded from the at least one subset of the plurality of supports to yield a plurality of processed supports (e.g., processed beads) that can comprise or consist of positive supports (e.g., comprising one or more template molecules, 1821c) and negative supports (e.g., not comprising template molecules, 1822c), resulting in an enrichment of template molecules.

In some cases, removal or degradation of the double-stranded nucleic acid molecules can be achieved using an enzyme. Such enzyme can be an exonuclease having 5′ to 3′ activity, e.g., an exonuclease III (ExoIII). The enzyme may be capable of processing blunt-ended double-stranded nucleic acid molecules, partially double-stranded nucleic acid molecules (e.g., with 5′ overhangs, with 3′ overhangs containing less than four bases), and/or nicked double-stranded nucleic acid molecules. For example, the ExoIII may selectively process those partially double-stranded nucleic acid molecules with 3′ overhangs containing less than four bases to remove them from the support, while leaving undisturbed the extended primer molecules (e.g., amplified first single-stranded nucleic acid molecules which are hybridized to second single-stranded nucleic acid molecules such that there is a large 3′ overhang). The second single-stranded nucleic acid molecules hybridized to the extended primer molecules may be denatured from the supports prior to subsequent processing (e.g., immobilization of the positive support, sequencing, etc.). Such processed supports may be subjected to a capture reaction and/or surface immobilization reactions using the template nucleic acid molecules or portions thereof, as described elsewhere herein, to isolate the positive supports from the negative supports.

Alternatively, with reference to FIG. 18D, the plurality of template nucleic acid molecules can be double-stranded nucleic acid molecules. The removal or degradation of unreacted surface primer molecules can enrich between positive supports and negative supports. Subsequent to nucleic acid extension reactions on the positive supports, and prior to a denaturation operation, the positive supports (e.g., 1851a) may comprise, coupled thereto, a plurality of double-stranded nucleic acid molecules (e.g., 1854), the double-stranded nucleic acid molecules corresponding to the template nucleic acid molecules. The negative supports (e.g., 1852a), which did not immobilize a template nucleic acid molecule, may comprise unreacted surface primer molecules (e.g., 1855). In some cases, positive supports may also comprise unreacted surface primer molecules (e.g., 1855). The mixture of positive supports and negative supports can be subjected to a reaction mixture comprising a single strand binding moiety which degrades or otherwise removes single-stranded nucleic acid molecules from both types of supports. For example, such removal or degradation can be achieved using an enzyme 1853, such as a nuclease specific to single-stranded nucleic acid molecules (e.g., ssDNA). Example exonucleases that can be used include exonuclease I (ExoI), Mung Bean nuclease (nuclease MB), exonuclease T (ExoT), and exonuclease VII (ExoVII). The unreacted (e.g., unamplified) surface primer molecules may be degraded or removed from the support to yield processed supports, where positive supports (e.g., 1851b) only retain the double-stranded nucleic acid molecules and the negative supports (e.g., 1852b) are bare of unreacted surface primer molecules. Such processed supports may be subjected to a capture reaction and/or surface immobilization reactions using the double-stranded nucleic acid molecules or portions thereof, as described elsewhere herein, to isolate the positive supports from the negative supports. Prior to such capture reaction and/or surface immobilization reactions, a denaturation operation may be performed to yield positive supports comprising, coupled thereto, single-stranded molecules which correspond to template nucleic acid molecules (or complements thereof) and washed. The single-stranded molecules which correspond to template nucleic acid molecules (or complements thereof) may be used during the capture and/or immobilization.

Alternatively, with reference to FIG. 18E, the plurality of template nucleic acid molecules can be single-stranded nucleic acid molecules. A plurality of supports, of which at least a subset of supports (e.g., 1861a) contains one or more template nucleic acid molecules (e.g., 1864) immobilized thereto via at least the first set of surface primer molecules, can be contacted with a reaction mixture that comprises a plurality of single-stranded nucleic acid molecules (e.g., 1870) that have at least partial sequence complementarity to the template nucleic acid molecules. For example, the template nucleic acid molecule can comprise a sequencing primer attachment sequence, and the plurality of single-stranded nucleic acid molecules can comprise sequencing primers. It will be appreciated that the template nucleic acid molecule can comprise any attachment sequence, and the plurality of single-stranded nucleic acid molecules can comprise a sequence corresponding to (e.g., complementary to) the attachment sequence. The sequencing primer attachment sequence may be at a 3′ end of the template nucleic acid molecules (e.g., distal to the support). Another subset of supports (e.g., 1862a) may not be coupled to any template nucleic acid molecule. The plurality of single-stranded nucleic acid molecules can be used to generate a plurality of double-stranded nucleic acid molecules (e.g., blunt-ended, partially double stranded) coupled to at least a subset of the plurality of supports. The plurality of double-stranded nucleic acid molecules can comprise at least a subset of the plurality of single-stranded nucleic acid molecules coupled (e.g., hybridized) to the template nucleic acid molecules. Thus, the plurality of single-stranded nucleic acid molecules can be used to convert the template nucleic acid molecules attached to the subset of supports (e.g., 1861a) to partially double stranded nucleic acid molecules, leaving the free and unbound (single-stranded) surface primer molecules (e.g., 1865) of the supports single-stranded. The resulting double-stranded nucleic acid molecules may have a 5′ overhang. In some instances, the resulting double-stranded nucleic acid molecules may have a 3′ overhang. Thereafter, the mixture of positive supports and negative supports can be subjected to a reaction mixture comprising a single strand binding moiety which degrades or otherwise removes single-stranded nucleic acid molecules from both types of supports. For example, such removal or degradation can be achieved using an enzyme 1863, such as a nuclease specific to single-stranded nucleic acid molecules (e.g., ssDNA). Example exonucleases that can be used include exonuclease I (ExoI), Mung Bean nuclease (nuclease MB), exonuclease T (ExoT), and exonuclease VII (ExoVII). The unreacted (e.g., unamplified) surface primer molecules (e.g., 1865) may be degraded or removed from the support to yield processed supports, where positive supports (e.g., 1861b) only retain the double-stranded nucleic acid molecules comprising the template nucleic acid molecules and the negative supports (e.g., 1856b) are bare of unreacted surface primer molecules. Such processed supports may be subjected to a capture reaction and/or surface immobilization reactions using the double-stranded nucleic acid molecules or portions thereof, as described elsewhere herein, to isolate the positive supports from the negative supports. Prior to such capture reaction and/or surface immobilization reactions, a denaturation operation may be performed to yield positive supports comprising, coupled thereto, single-stranded molecules which correspond to template nucleic acid molecules (or complements thereof) and washed. The single-stranded molecules which correspond to template nucleic acid molecules (or complements thereof) may be used during the capture and/or immobilization.

In various embodiments herein, methods for enrichment of positive supports can comprise the use of modified nucleotides for immobilization of such positive supports on surfaces prior to analysis, e.g., prior to sequencing. Such process can be referred to herein as pre-enrichment and can include chemoselective immobilization of positive supports on a surface. In some cases, the positive supports (e.g., beads) can further comprise one or more functionalities that enable attachment (e.g., immobilization) of such supports to a substrate surface. Surface attachment or immobilization of a mixture of supports that can comprise positive and negative supports (e.g., beads) can thus be used to enrich or select for such positive supports. In such instances, a mixture of positive and negative supports can be enriched on a surface using various methods described herein, or variations thereof. In some embodiments, a mixture of positive and negative supports is subjected to contact liquid or solution comprising a polymerase (e.g., a DNA polymerase) and one or more modified nucleotides, wherein two or more modified nucleotides can comprise different nucleobases and/or different reactive moieties. Such modified nucleotides can comprise a reactive moiety that is capable of interacting (e.g., covalent or non-covalent binding) with a second reactive moiety on a surface. For example, the reactive moiety of a modified nucleotide can be an azide moiety capable of reacting with a cyclooctene moiety attached to the surface of a substrate. In another example, the reactive moiety of a modified nucleotide can be a biotin moiety capable of interacting with streptavidin moiety attached to the substrate surface. Other pairs of reactive chemical moieties can also be used, including other bioconjugation or biorthogonal conjugation methodologies, as described herein. In the mixture comprising the positive and negative supports, polymerase(s), and modified nucleotide(s), the polymerase can enzymatically incorporate one or more modified nucleotides into the strand that is complementary to the template sequence and that is being generated on the surface of the support (e.g., bead). In some cases, at least 1, 2, 3, 4, 5, 6, 7, 8, 9, 10, 15, 20, 30, or more modified nucleotides can be incorporated into a complementary template strand during elongation. Such one or more modified nucleotides can either be identical or different, e.g., identical nucleotides comprising different reactive moieties, different nucleotides comprising identical reactive moieties, or different nucleotides comprising different reactive moieties. Because incorporation of modified nucleotides into a support-template construct depends on the presence of a template nucleic acid molecule coupled to the support, only positive supports are likely to comprise modified nucleotides and are subsequently immobilized on the substrate surface.

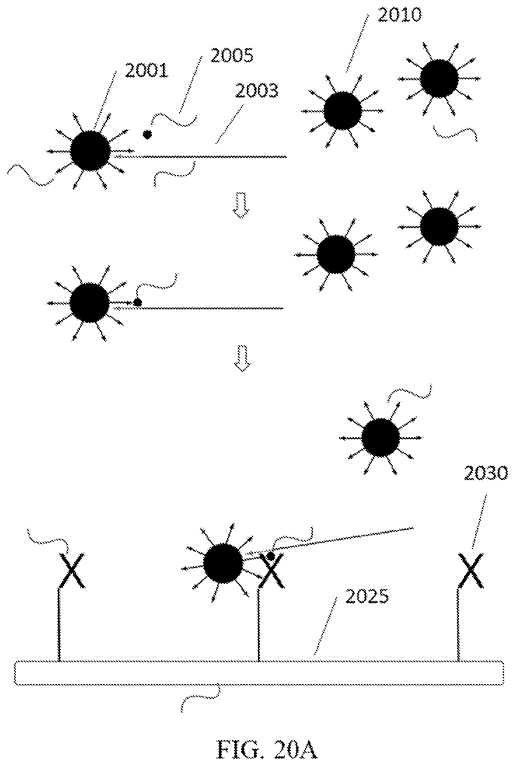

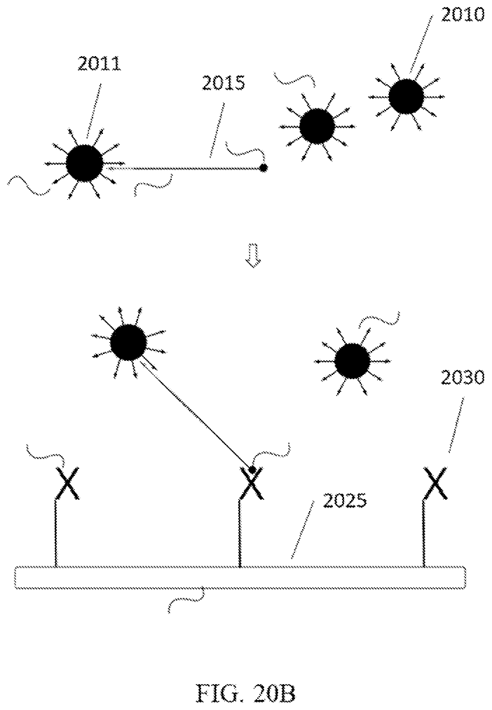

An example of such approach is illustrated in FIG. 20A. Positive support 2001 comprising the template sequence 2003 can be used to incorporate a modified nucleotide 2005 in the complementary strand. Positive support 2001 may comprise a support primer hybridized to a known adapter sequence of the template sequence 2003. The adapter sequence may comprise a first portion and a second portion, the first portion for hybridization with the support primer molecule, and the second portion designed to permit incorporation of the modified nucleotide at a known position. Beneficially, the position of the incorporation of the modified nucleotide may be known and selected. Subsequently, the mixture comprising the modified support-template constructs can be subjected to a surface 2025 comprising complementary reactive moieties 2030 capable of interacting with the reactive moiety on the modified nucleotide integrated into the support-template construct. Thus, positive supports can be selectively immobilized and thereby enriched on the surface, whereas negative supports 2010 can be separated from the positive supports, e.g., in a washing step. The immobilized supports comprising the template sequences can then be subjected to conditions sufficient to extend or amplify (or further amplify) the template nucleic acid sequences followed by analysis, e.g., sequencing. The modified support-template constructs may be immobilized to the surface immediately following incorporation of the modified nucleotide, such as prior to incorporation of the next nucleotide. Beneficially, the modified nucleotide may be incorporated at a relatively early position (e.g., proximal to the bead, and corresponding to the second portion of the template adapter sequence) for early capture of the support to the surface for subsequent processing. Alternatively, the modified support-template constructs may be immobilized to the surface at any point subsequent to incorporating the modified nucleotide.

In another embodiment herein, the template nucleic acid molecules of a sample can themselves be modified to comprise one or more reactive moieties such that, upon hybridization of a template sequence to a surface primer of a support (e.g., a bead), the support-template construct can be immobilized on a substrate surface. Such method may circumvent the use of polymerizing enzymes and polymerase steps and may merely require attachment of the modified template molecules to supports. Various methods to incorporate one or more modified nucleotides into a template nucleic acid molecule can be envisioned. In one embodiment, an adapter sequence comprising one or more modified nucleotides can be coupled to either the 3′- and/or the 5′-end of the template molecule. In some instances, such adapter sequence is coupled to the 5′-end of the template molecule. Following attachment of the modified template nucleic acid molecules to supports (e.g., beads), the resulting positive supports can be immobilized to a surface via the reactive moieties attached to the 5′-end of the template molecules.

An example of such approach is illustrated in FIG. 20B. Positive support 2011 comprising the template sequence 2015, which comprises a reactive moiety, can be subjected to a surface 2025 comprising complementary reactive moieties 2030 capable of interacting with the reactive moiety on the modified nucleotide of the template sequence. Thus, positive supports can be selectively immobilized and thereby enriched on the surface, whereas negative supports 2010 can be separated from the positive supports, e.g., in a washing step. The immobilized supports comprising the template sequences can then be subjected to conditions sufficient to extend or amplify (or further amplify) the template nucleic acid sequences followed by analysis, e.g., sequencing.

In some instances, the efficiency of a method for immobilizing positive supports on a surface can be increased by incorporating a plurality of modified nucleotides, either during generation of a complementary strand (e.g., primer extension), or via direct coupling (e.g., covalent coupling) to the template sequence. As described elsewhere herein, such plurality of modified nucleotides can include 2, 3, 4, 5, 6, 7, 8, 9, 10, 15, 20, 30, 50, 100, or more modified nucleotides. In some instances, a plurality of nucleotides is incorporated during primer extension such that continuous segments of modified nucleotides are generated, wherein such segments can comprise 2, 3, 4, 5, 6, 7, 8, 9, 10, or more contiguous nucleotides. In other instances, a plurality of nucleotides is incorporated during primer extension such that each modified nucleotide is separated from another modified nucleotide by at least one unmodified nucleotide. Furthermore, incorporation of modified nucleotides can occur closer to the support (e.g., bead) surface or closer to the distal end of the complementary template sequence being generated during primer extension, or in a combination thereof. In some instances, incorporation of modified nucleotides closer to the middle and/or distal end of the complementary template can increase the steric accessibility of the reactive moiety by moieties attached to a substrate surface, and thereby increase surface immobilization of positive supports.

In some instances, amplification of template nucleic acid molecules may commence after selective immobilization of the positive supports onto the surface. Such selective immobilization may have enriched for positive supports from a mixture of positive supports and negative supports, as described elsewhere herein. In some instances, the efficiency of the on-surface amplification may be increased by using the unique support species as described elsewhere herein. Beneficially, neighboring supports immobilized to the surface will likely be of different species such that an amplification product derived from a first template associated with a first support is incapable of coupling to the different support species in the immediate vicinity and more likely to couple to the first support. Alternatively or in addition, the viscosity of the solution may be increased, such as by providing a crowding agent or alternative, e.g., PEG, to decrease migration activity of amplification products between different supports. In some cases, the amplification of template nucleic acid molecules herein can comprise performing recombinase polymerase amplification (RPA). The RPA may further comprise use of a crowding agent or alternative, such as a polyethylene glycol (PEG), or a derivative thereof. Alternatively or in addition, the surface may comprise physical features that facilitate containment of amplification products to the originating support. For example, the surface may be patterned with recesses and/or ridges that prevents or impedes an amplification product form traveling from one recess (comprising a support) to another recess (comprising a different support).

In addition to the above described methods that can comprise amplification of immobilized template molecules attached to supports (e.g., beads), the present disclosure further provides methods for amplification of single template nucleic acid molecules. Such methods can comprise attaching primers to a plurality of template nucleic acid molecules in free solution, i.e., not bound to any support (e.g., bead). As described herein, such process can result in a Poisson distribution of non-hybridized primers and the primer-template constructs. However, using such process, no higher order hybrids (e.g., beads with two or more different template molecules attached) can be formed. For the amplification step, a mixture of a polymerase (e.g., a DNA polymerase) and modified dNTPs that can comprise one or more biorthogonal function groups for subsequent immobilization on a surface can be added to the solution comprising free, unreacted primers and primers hybridized to template molecules. The 3′ primer extension reaction can then be completed, and any non-extended, unmodified primers, excess of non-incorporated dNTP, and polymerase can then be removed, once the extended double-stranded template-primer hybrids are attached to a surface. The double-stranded target molecules can be attached to such surface using any of the biorthogonal coupling strategies described herein, e.g., biotin-streptavidin, azide-cyclooctyne, etc.

### Enrichment Prior to Partitioning or Surface-Loading (Pre-Enrichment)

Provided herein are methods for generating a pre-assembled support, generally referred to herein as an assembly, wherein the assembly comprises a single template nucleic acid molecule immobilized to a single support. In subsequent operations, such assemblies, in collection, may be partitioned, as described herein, together with amplification reagents (e.g., solution primer) to facilitate amplification reactions of the template nucleic acid molecules within individual reaction chambers of an emulsion. Beneficially, a partition comprising a single assembly may immobilize a monoclonal population of amplification products to the same support within the partition. Compartmentalization or encapsulation of such assemblies in partitions may follow a Poisson distribution to include, for example, in addition to partitions comprising a single assembly, partitions not comprising any assemblies, and/or partitions comprising a plurality of assemblies (e.g., with different template sequences). By providing pre-assembled supports prior to partitioning, beneficially, a double Poisson problem for distribution amongst partitions of an emulsion may be reduced to a single Poisson distribution problem. If a plurality of supports and a plurality of templates (not immobilized to the supports) are partitioned, each following its own Poisson distribution model, significantly fewer partitions having a single support and a single template are generated compared to a first order Poisson distribution model. This can result in inefficient use of valuable resources and loss of precious templates.

In some instances, a method may comprise providing a mixture comprising a plurality of extended supports and a plurality of template nucleic acid molecules (e.g., in a library) each having a different nucleic acid sequence. The plurality of extended supports may comprise a purified composition of the extended supports (from a mixture of extended supports and un-extended supports) as described elsewhere herein. Each of the template nucleic acid molecules may be configured to, and/or be capable to, anneal with a second primer. An extended support of the plurality of extended supports may comprise a plurality of first primers and a single copy, a few copies, several copies, and/or a significantly lower number of the second primers relative to a number of the plurality of first primers available for annealing to the template nucleic acid molecules. The mixture may be subject to conditions sufficient to anneal or otherwise associate the plurality of template nucleic acid molecules to a plurality of second primers distributed across the plurality of extended supports. The mixture may be subject to conditions sufficient to wash template nucleic acid molecules that have not coupled to a support. In some instances, this may be achieved by immobilizing the support to an immobilization platform (e.g., another surface or structure configured to immobilize the support, such as via some affinity (e.g., magnetic, electric, hydrophobic, hydrophilic, etc.) to the support, etc.) such that during washing the support remains stabilized. Because each extended support has only one copy, a few copies, several copies, and/or a significantly lower number of the second primers relative to a number of the plurality of first primers, the resulting reaction products may comprise a plurality of assemblies, wherein a majority of, or substantially all of, the assemblies each comprise a single template nucleic acid molecule immobilized to a support. Such assemblies may be partitioned, as described elsewhere herein, such as together with amplification reagents (e.g., including a solution primer) to facilitate amplification reactions of the template nucleic acid molecule within individual reaction chambers. Beneficially, a partition comprising a single assembly may immobilize a monoclonal population of amplification products to the same support within the partition.

In some examples, during the mixing process of extended supports and the template nucleic acid molecules, the concentration of extended supports may be lower than the concentration of the template nucleic acid molecules, where suitable (e.g., when there is an abundance of available sample). For example, sample(s) may be provided in excess. Providing the sample in excess may reduce the number of blank extended supports (lacking templates) resulting from mixing and hybridization.

Using some methods, a technician may have to make very precise measurements to prepare a sample, such as prior to mixing the sample with the supports to generate a useful population of assemblies. The extended supports provided in this disclosure may advantageously be compatible with processes that do not require as precise measurements of the concentration of the library in a reaction mixture. In some examples, merely providing the sample in excess may contribute to a successful hybridization (e.g., to extended supports) process, even when the concentration of the library is not measured with high precision. For example, in some cases, providing the sample in excess may allow decrease of the incubation time for hybridization reaction. Providing the sample (library) in excess may increase the rate of hybridization and yield. Alternatively, in some cases the sample may not be provided in excess (for example in cases where the sample is precious).

In some instances, referring to FIG. 12A, a method may comprise providing a mixture comprising a plurality of supports (e.g., support 1201, un-extended support) and a plurality of template nucleic acid molecules (e.g., template nucleic acid molecule 1202) each having a different nucleic acid sequence. The template nucleic acid molecule may be configured to, and/or be capable to, anneal with a primer attached to the support. The template nucleic acid molecule may comprise a capture entity 1206 configured for subsequent capture by a capturing entity of a capturing group 1208. The support may comprise a plurality of primers available for annealing to the template nucleic acid molecules.

Alternatively, FIG. 12B illustrates an example method wherein the template nucleic acid molecule lacks a capture entity 1206, and the capture entity 1206 is added to the extension product. As described with respect to FIG. 12A, a mixture comprising a plurality of supports (e.g., support 1201, un-extended support) and a plurality of template nucleic acid molecules (e.g., template nucleic acid molecule 1202) each having a different nucleic acid sequence are provided. The template nucleic acid molecule lacking a capture entity may be configured to, and/or be capable to, anneal with a primer attached to the support. Similar to the description of FIG. 10B, a primer may attach to the template nucleic acid molecule, for example via hybridization of complementary sequences (e.g., between a sequence of the first primer and a sequence of adapter 1), and subsequently extended to generate an extension product that is immobilized to the support. For the extension reaction, reagents comprising the capture entity (e.g., nucleotides comprising the capture entity) may be used resulting in the extension product comprising the capture entity 1206. In some instances, the capture entity may be biotin (B), such that biotin labeled nucleotides are used for the extension reaction. A single labeled base may be employed, such as labeled adenine, labeled thymine, labeled guanine, or labeled cytosine, or analogs thereof. The labeled nucleotide may be selected based on the sequence of adapterl of the template 1202. In an example, only a single labeled nucleotide is added. This can be achieved by performing the extension in two operations. In the first operation, only the first nucleotide is added, and this nucleotide is labeled with the capture entity 1206. This can be the first base present in a sequence of adapter 1 that is not complementary to a sequence of the first primer. A second extension reaction is performed with all the bases, wherein no labeled bases are used. This results in an extension product immobilized to the support comprising only one capture entity 1206. Alternatively, the stepwise single labeled nucleotide addition can be performed at any other position of the extension (e.g., second position, third position, fourth position, etc.).

In some instances, the respective concentrations of the supports and the template nucleic acid molecules in a reaction mixture may be modulated to facilitate generation of a majority of assemblies comprising a single support and a single template nucleic acid molecule (or complement thereof) immobilized to the support. For example, the resulting support may comprise at least about 50%, 60%, 70%, 80%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, 99.9999% or more of the primer not associated with a template nucleic acid molecule (out of the total primer population).

For example, the reaction mixture may contain a fewer number or less concentration of the template nucleic acid molecules relative to the number or concentration of the supports present. In some instances, the ratio of a concentration of template nucleic acid molecules to a concentration of supports in a solution is at most about 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50 or less. Alternatively or in addition, the ratio of a concentration of template nucleic acid molecules to a concentration of supports in a solution is at least about 1:50, 1:40, 1:30, 1:20, 1:29, 1:18, 1:17, 1:16, 1:14, 1:13, 1:12, 1:11, 1:10, or greater. In some instances, the percentage of a concentration of template nucleic acid molecules to a concentration of supports in a solution is at most about 50%, 45%, 40%, 35%, 30%, 25%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less. Alternatively or in addition, the percentage of a concentration of template nucleic acid molecules to a concentration of supports in a solution is at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or greater.

Referring back to FIG. 12A, the mixture may be subject to conditions sufficient to anneal (1203) the plurality of template nucleic acid molecules to a plurality of primers distributed across the plurality of supports, and subject to extension (1204) to generate complements of the respective template nucleic acid molecules immobilized thereto the respective supports. The supports may remain associated with the respective capture entities (e.g., 1206) of the respective template nucleic acid molecules (e.g., 1202). A resulting mixture may comprise a mixture of supports comprising one or more template nucleic acid molecules (and capture entities) associated thereto and supports not comprising any template nucleic acid molecules (and capture entities) associated thereto.

In some instances, the capture entity 1206 may comprise biotin (B). In some instances, the capture entity may comprise a capture sequence (e.g., nucleic acid sequence). In some instances, a sequence of the template nucleic acid molecule may function as a capture sequence. In other instances, the capture entity may comprise another nucleic acid molecule comprising a capture sequence. In some instances, the capture entity may comprise a magnetic particle capable of capture by application of a magnetic field. In some instances, the capture entity may comprise a charged particle capable of capture by application of an electric field. In some instances, the capture entity may comprise one or more other mechanisms configured for, or capable of, capture by a capturing entity, as described elsewhere herein.

The support 1201 comprising the template nucleic acid molecule 1202 associated thereto may be brought in contact with, or otherwise subjected to capture by, a capturing group 1208. The capturing group may comprise a capturing entity configured to capture the capture entity 1206. For example, the capturing entity may be configured to target the capture entity. In some instances, the capturing entity may comprise streptavidin (SA) when the capture moiety comprises biotin. In some instances, the capturing entity may comprise a complementary capture sequence when the capture entity comprises a capture sequence (e.g., that is complementary to the complementary capture sequence). In some instances, the capturing entity may comprise an apparatus, system, or device configured to apply a magnetic field when the capture entity comprises a magnetic particle. In some instances, the capturing entity may comprise an apparatus, system, or device configured to apply an electrical field when the capture entity comprises a charged particle. In some instances, the capturing entity may comprise one or more other mechanisms configured to capture the capture entity. In some instances, the capturing group may comprise a secondary capture entity, for example, for subsequent capture by a secondary capturing entity 1207. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

When the support comprising the capture entity 1206 associated thereto is brought in contact with, or otherwise subject to capture by, the capturing group 1208, the capturing entity of the capturing group may bind, couple, hybridize, or otherwise associate with the capture entity. The association between the capture entity and the capturing entity may comprise formation of a non-covalent bond. The association may comprise formation of a covalent bond. The association may comprise formation of a releasable bond, for example, upon application of a stimulus. In some instances, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities.

In some instances, the capturing group 1208 may isolate the support 1201 comprising the template nucleic acid molecule 1202 (and capture entity 1206) from a mixture by targeting the capture entity. In some instances, the capturing group may isolate multiple supports each comprising one or more template nucleic acid molecules from a mixture. In some instances, a plurality of capturing groups may be used to isolate the support comprising the template nucleic acid molecule from a mixture. Once isolated, a washing and/or melting operation (1205) may be performed to disassociate the template nucleic acid molecule from the support to provide the assembly 1200.

In some instances, the capturing group 1208 may associate with the support without isolation of the support from the mixture. In some instances, where the capturing group further comprises a secondary capture entity, the support may remain associated with the secondary capture entity in the mixture. The support may be brought into contact with, or otherwise subject to capture by, a secondary capturing entity 1207. The secondary capturing entity may bind, couple, hybridize, or otherwise associate with the secondary capture entity of the capturing group. The association between the secondary capture entity and the secondary capturing entity may comprise formation of a non-covalent bond. The association may comprise formation of a covalent bond. The association may comprise formation of a releasable bond, for example, upon application of a stimulus. In some instances, the association may not form any bond. For example, the association may increase a physical proximity (e.g., decrease physical distance) of the secondary capturing entity and secondary capture entity. In some instances, a single secondary capture entity may be capable of associating with a single secondary capturing entity. Alternatively, a single secondary capture entity may be capable of associating with multiple secondary capturing entities. Alternatively or in addition, a single secondary capturing entity may be capable of associating with multiple secondary capture entities. In some instances, the secondary capturing group may isolate the support comprising the template nucleic acid molecule from a mixture. In some instances, the secondary capturing group may isolate multiple supports from a mixture. In some instances, a plurality of secondary capturing groups may be used to isolate the support from a mixture.

Once isolated, a washing and/or melting operation may be performed to disassociate the template nucleic acid molecule 1202 and the capture group 1208 (and in some cases also the secondary capturing entity 1207) from the support to provide the assembly 1200.

In some instances, the secondary capturing entity 1207 may associate with the support without isolation of the support from the mixture. In some cases, the secondary capturing entity may comprise a third capture entity configured for subsequent capture by a third capturing entity (not illustrated). It will be appreciated that any degree of capturing entity may comprise another capture group that may be captured by a next degree of capturing entity, for isolation from the mixture and/or association by the next degree of capturing entity. Once isolated, a washing and/or melting operation may be performed to disassociate the template nucleic acid molecule (and any number of capture entities and/or capturing entities) from the support to provide the assembly 1200.

Such assemblies may be partitioned, as described elsewhere herein, such as together with amplification reagents (e.g., including a solution primer) to facilitate amplification reactions of the template nucleic acid molecule within individual reaction chambers. Beneficially, a partition comprising a single assembly may immobilize a monoclonal population of amplification products to the same support within the partition.

Methods for pre-enrichment of the supports (with template nucleic acid molecules or complements thereof) may be performed in solution. In some examples, the pre-enrichment methods may be performed in a solution not comprising any emulsion or partitions. In other examples, the pre-enrichment method may be performed in partitions. Procedures may be integrated. Alternatively, processes may not be integrated.

### On-Wafer Enrichment

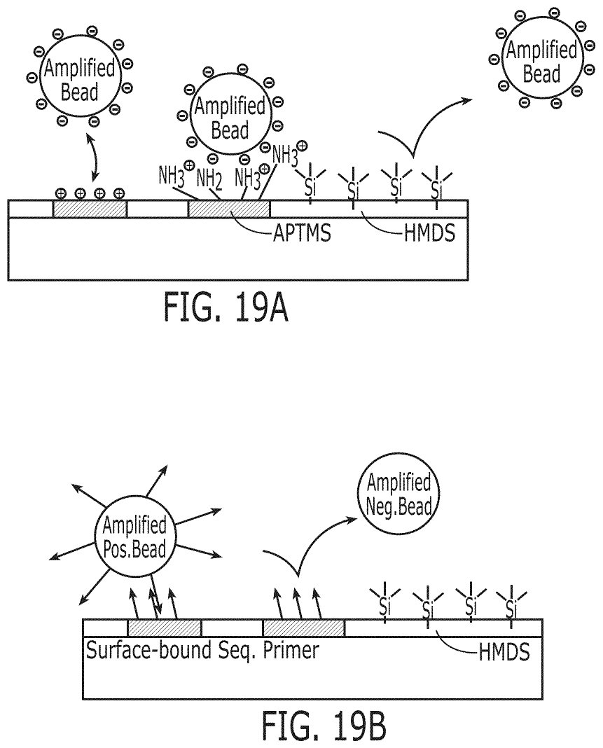

In some cases, an individually addressable location may comprise a distinct surface chemistry. The distinct surface chemistry may distinguish between different addressable locations. The distinct surface chemistry may distinguish between different regions. For example, a first location has a first affinity towards an object (e.g., a bead comprising nucleic acid molecules, e.g., amplicons, immobilized thereto, e.g., a positive bead) and a second location has a second, different affinity towards the object due to the distinct surface chemistries. The first location and the second location may or may not be located in the same region. The first location and the second location may or may not be disposed on the surface in alternating fashion. In another example, a first region (e.g., comprising a plurality of individually addressable locations) has a first affinity towards an object and a second region has a second, different affinity towards the object due to the distinct surface chemistries. A first location type or region type may comprise a first surface chemistry, and a second location type or region type may comprise a second surface chemistry. In some cases, a third location type or region type may comprise a third surface chemistry. For example, a first location type or region type may comprise a positively charged surface chemistry and/or a hydrophobic surface chemistry, and a second location type or region type may comprise a negatively charged surface chemistry and/or a hydrophilic surface chemistry, as shown in FIG. 19A. The same object (e.g., a bead comprising nucleic acid molecules, e.g., amplicons, immobilized thereto, e.g., a positive bead) may have higher affinity towards a first location type or region type compared to a second location type or region type. The same object may be attracted towards a first location type or region type and repelled from a second location type or region type. In other examples, a first location type or region type comprising a first surface chemistry (e.g., a positively charged surface chemistry or a negatively charged surface chemistry) may interact with (e.g., have an affinity towards) a first sample type (e.g., a bead comprising nucleic acid molecules, e.g., amplicons, immobilized thereto, e.g., a positive bead) and exclude a second sample type (e.g., a bead lacking nucleic acid molecules, e.g., amplicons, immobilized thereto, e.g., entirely or in substantial volume, e.g., a negative bead), for example as illustrated in FIG. 19B. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl) triethoxysilane (APTMS). In some cases, the surface chemistry may comprise a surface primer molecule (e.g., a sequencing primer molecule) or any oligonucleotide molecule that has any degree of affinity towards another molecule.

An individually addressable location of a plurality of locations (e.g., alternating locations) may have an area. In some cases, a location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², or about 6 μm². A location may have an area that is within a range defined by any two of the preceding values. A location may have an area that is less than about 0.1 μm² or greater than about 6 μm². In some cases, a location may have a width of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, or about 6 μm. In some cases, a location may have a width that is within a range defined by any two of the preceding values. A location may have a width that is less than about 0.1 μm or greater than about 6 μm. Locations (e.g., of a same type) may be distributed on a substrate with a pitch determined by the distance between the center of a first location and the center of the closest or neighboring location (e.g., of the same type). Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some case the locations may be positioned with a pitch that is within a range defined by any two of the preceding values. The locations may be positioned with a pitch of less than about 0.1 μm or greater than about 10 μm. In some cases, the pitch between any two locations of the same type may be determined as a function of a size of a loading object (e.g., bead). For example, where the loading object is a bead having a maximum diameter, the pitch may be at least about the maximum diameter of the loading object.

While examples herein generally describe the loading of two samples or two sets of samples, any number of samples, or sets of samples, may be immobilized to the substrate. For example, the substrate may have immobilized thereto at least 1, at least 2, at least 3, at least 4, at least 5, at least 6, at least 7, at least 8, at least 9, at least 10 samples, or sets of samples. In some cases, at least about 10, 100, 1000, 10,000, 100,000, 1,000,000, 10,000,000, 100,000,000, 1,000,000,000 or more samples, or sets of samples, may be immobilized. Alternatively or in addition, the substrate may comprise at most about 1,000,000,000, 100,000,000, 10,000,000, 1,000,000, 100,000, 10,000, 1000, 100, 10 or fewer samples, or sets of samples. When the sample is a nucleic acid sample, at least 1, at least 2, at least 3, at least 4, at least 5, at least 6, at least 7, at least 8, at least 9, at least 10 nucleic acid samples may be immobilized to the substrate. In some cases, at least about 10, 100, 1000, 10,000, 100,000, 1,000,000, 10,000,000, 100,000,000, 1,000,000,000 or more nucleic acid samples may be immobilized. Alternatively or in addition, the substrate may comprise at most about 1,000,000,000, 100,000,000, 10,000,000, 1,000,000, 100,000, 10,000, 1000, 100, 10 or fewer nucleic acid samples. Beneficially, multiple samples may be simultaneously processed on the same substrate, without needing to otherwise barcode the multiple samples (e.g., with a common barcode sequence per sample) to distinguish them.

Indexing may be performed using a detection method and may be performed at any convenient or useful step. A substrate that is indexed, e.g., demarcated, may be subjected to detection, such as optical imaging, to locate the indexed locations, individually addressable locations, and/or the biological analyte. Imaging may be performed using a detection unit. Imaging may be performed using one or more sensors. Imaging may not be performed using the naked eye. The substrate that is indexed may be imaged prior to loading of the biological analyte. Following loading of the biological analyte onto the individually addressable locations, the substrate may be imaged again, e.g. to determine occupancy or to determine the positioning of the biological analyte relative to the substrate. In some cases, the substrate may be imaged after iterative cycles of nucleotide addition (or other probe or other reagent), as described elsewhere herein. The indexing of the substrate and known initial position (individually addressable location) of the biological analyte may allow for analysis and identification of the sequence information for each individually addressable location and/or position. Additionally, spatial indexing may allow for identification of errors that may occur, e.g., sample contamination, sample loss, etc.

In some cases, indexing may be performed to identify, process, and/or analyze more than one type of biological analyte, as described above. For example, a first type of biological analyte, which may be labeled, may be loaded onto a first set of locations within a substrate. The substrate may be imaged for a first indexing step of the first type of biological analyte. A second type of biological analyte may be loaded onto a second set of locations within the substrate and imaged for a second indexing step of the second type of biological analyte. In some cases, the second type of biological analyte may be labeled in a way such that the second type of biological analyte is distinguishable from the first type of biological analyte. Alternatively, the first type of biological analyte and the second type of biological analyte may be labeled in substantially the same detectable manner (e.g., same dye), and the first and second images may be processed to generate a differential image, wherein overlapping signals are attributed to the locations of the first type of biological analyte and different signals are attributed to the locations of the second type of biological analyte. Alternatively, the first type of biological analyte and the second type of biological analyte may be labeled by cleavable (or otherwise removable) labels or tags (e.g., fluorescent tags), and the label cleaved after each imaging operation, such that only the relevant analyte locations are imaged at each imaging operation. Henceforth, the substrate may be analyzed and all of the locations comprising the first biological analyte may be attributed to the first biological analyte, and all of the locations comprising the second biological analyte may be attributed to the second analyte. In some cases, labeling of the first and second analyte may not be necessary, and the attribution of the location to either the first or second analyte may be performed based on spatial location alone. This process may be repeated for any number or types of biological analytes.

The array may be coated with binders. For instance, the array may be randomly coated with binders. Alternatively, the array may be coated with binders arranged in a regular pattern (e.g., in linear arrays, radial arrays, hexagonal arrays etc.). The array may be coated with binders on at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the number of individually addressable locations, or of the surface area of the substrate. The array may be coated with binders on a fraction of individually addressable locations, or of the surface areas of the substrate, that is within a range defined by any two of the preceding values. The binders may be integral to the array. The binders may be added to the array. For instance, the binders may be added to the array as one or more coating layers on the array.

The binders may immobilize biological analytes through non-specific interactions, such as one or more of hydrophilic interactions, hydrophobic interactions, electrostatic interactions, physical interactions (for instance, adhesion to pillars or settling within wells), and the like. The binders may immobilize biological analytes through specific interactions. For instance, where the biological analyte is a nucleic acid molecule, the binders may comprise oligonucleotide adapters configured to bind to the nucleic acid molecule. Alternatively or in addition, such as to bind other types of analytes, the binders may comprise one or more of antibodies, oligonucleotides, nucleic acid molecules, aptamers, affinity binding proteins, lipids, carbohydrates, and the like. The binders may immobilize biological analytes through any possible combination of interactions. For instance, the binders may immobilize nucleic acid molecules through a combination of physical and chemical interactions, through a combination of protein and nucleic acid interactions, etc. The array may comprise at least about 10, 100, 1000, 10,000, 100,000, 1,000,000, 10,000,000, 100,000,000 or more binders. Alternatively or in addition, the array may comprise at most about 100,000,000, 10,000,000, 1,000,000, 100,000, 10,000, 1000, 100, 10 or fewer binders. The array may have a number of binders that is within a range defined by any two of the preceding values. In some instances, a single binder may bind a single biological analyte (e.g., nucleic acid molecule). In some instances, a single binder may bind a plurality of biological analytes (e.g., plurality of nucleic acid molecules). In some instances, a plurality of binders may bind a single biological analyte. Though examples herein describe interactions of binders with nucleic acid molecules, the binders may immobilize other molecules (such as proteins), other particles, cells, viruses, other organisms, or the like.

In some instances, each location, or a subset of such locations, may have immobilized thereto an analyte (e.g., a nucleic acid molecule, a protein molecule, a carbohydrate molecule, etc.). In other instances, a fraction of the plurality of individually addressable location may have immobilized thereto an analyte. A plurality of analytes immobilized to the substrate may be copies of a template analyte. For example, the plurality of analytes (e.g., nucleic acid molecules) may have sequence homology. In other instances, the plurality of analytes immobilized to the substrate may not be copies. The plurality of analytes may be of the same type of analyte (e.g., a nucleic acid molecule) or may be a combination of different types of analytes (e.g., nucleic acid molecules, protein molecules, etc.).

In some instances, the array may comprise a plurality of types of binders. For example, the array may comprise different types of binders to bind different types of analytes. For example, the array may comprise a first type of binders (e.g., oligonucleotides) configured to bind a first type of analyte (e.g., nucleic acid molecules), and a second type of binders (e.g., antibodies) configured to bind a second type of analyte (e.g., proteins), and the like. In another example, the array may comprise a first type of binders (e.g., first type of oligonucleotide molecules) to bind a first type of nucleic acid molecules and a second type of binders (e.g., second type of oligonucleotide molecules) to bind a second type of nucleic acid molecules, and the like. For example, the substrate may be configured to bind different types of analytes in certain fractions or specific locations on the substrate by having the different types of binders in the certain fractions or specific locations on the substrate.

A biological analyte may be immobilized to the array at a given individually addressable location of the plurality of individually addressable locations. An array may have any number of individually addressable locations. For instance, the array may have at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, or at least 100,000,000,000 individually addressable locations. The array may have a number of individually addressable locations that is within a range defined by any two of the preceding values. Each individually addressable location may be digitally and/or physically accessible individually (from the plurality of individually addressable locations). For example, each individually addressable location may be located, identified, and/or accessed electronically or digitally for mapping, sensing, associating with a device (e.g., detector, processor, dispenser, etc.), or otherwise processing. As described elsewhere herein, each individually addressable location may be indexed. Alternatively, the substrate may be indexed such that each individually addressable location may be identified during at least one step of the process. Alternatively or in addition, each individually addressable location may be located, identified, and/or accessed physically, such as for physical manipulation or extraction of an analyte, reagent, particle, or other component located at an individually addressable location.

In an aspect, the present disclosure provides a method for analyte detection or analysis comprising providing an open substrate comprising a central axis (e.g., as described herein). The open substrate may be, for example, a wafer or disc, such as a wafer or disc having one or more substances patterning its surface. The open substrate may be substantially planar. The open substrate may have an array of immobilized analytes thereon (e.g., as described herein). The immobilized analytes may be immobilized to the array via one or more binders. The array may comprise at least 100,000 such binders. In some cases, an immobilized analyte of the immobilized analytes may be coupled to a bead, and the bead may be immobilized to the array. An immobilized analyte may comprise a nucleic acid molecule.

### IV. Attachment Mechanisms of Supports to Surfaces

Provided herein are methods and compositions for attaching (or immobilizing) a support (or a plurality of supports such as a plurality of beads) to a substrate surface (e.g., a planar array). The support can be a bead. In order to attach a support to a surface, various bioconjugation chemistries can be used. In some cases, such bioconjugation chemistries can be biorthogonal.

In some instances, a support (e.g., a bead) can be immobilized to a surface by non-covalent interactions. Examples of such non-covalent interactions include nucleic acid hybridization and biotin-streptavidin interaction. In some cases, a support (e.g., a bead) can be immobilized to a surface by nucleic acid hybridization. In an example, as shown in FIG. 19B, a positive bead comprising single-stranded template nucleic acid molecules attached to its surface can be immobilized to a substrate surface by hybridization of a single-stranded template nucleic acid molecule, or portion thereof, with a single-stranded, at least partially complementary, nucleic acid molecule that is attached to the substrate surface. In other cases, a support (e.g., a bead) can be immobilized to a surface by biotin-streptavidin interaction. In an example, as shown in FIG. 1, a template nucleic acid molecule of a sample can comprise a biotin moiety at one of its ends, e.g., at the 3′ end. Upon hybridizing to a surface primer that is attached to the surface of a support (e.g., a bead), the support can be immobilized on a substrate surface comprising streptavidin moieties via interaction of the biotin moiety of the template molecule with a streptavidin moiety of the substrate surface.

In various aspects herein, a support (e.g., a bead) can be immobilized to a surface by covalent interactions. Such covalent interactions can include various bioconjugation chemistries such as thiol-maleimide interactions, and click chemistries, such as azide-cyclooctyne, and tetrazine-trans-cyclooctene. In an example, a solution of a polymerase (e.g., a DNA polymerase) and a modified deoxy nucleotide triphosphate (dNTP) can be added to the mixture. The dNTP added can be modified with a chemical moiety such as biotin, an azide, a cyclooctyne, tetrazole, a thiol, or a similar functional group that allows for bioconjugation of the beads to a surface. Furthermore, the dNTP can be chosen based on the sequence of the primer-template hybrid expected to be formed upon the hybridization reaction. For example, if the next base on the template nucleic acid molecule downstream from the 3′ end of the capture primer is a G, then the added modified dNTP can be a complementary “C”; on the other hand, if the next base is an A, the modified dNTP can be a complementary T, and so on. The polymerase can enzymatically incorporate one (or more) of these nucleotides onto the 3′ end of the bead-bound primer, but only in cases where a suitable complementary target DNA had been captured by hybridization. Thus, no or no significant incorporation of these modified dNTPs is likely to occur in cases of “empty” beads (i.e., beads that do not contain any template molecules or amplicons thereof). Any excess non-incorporated dNTPs can be removed from the mixture before proceeding.

### V. Computer Control Systems

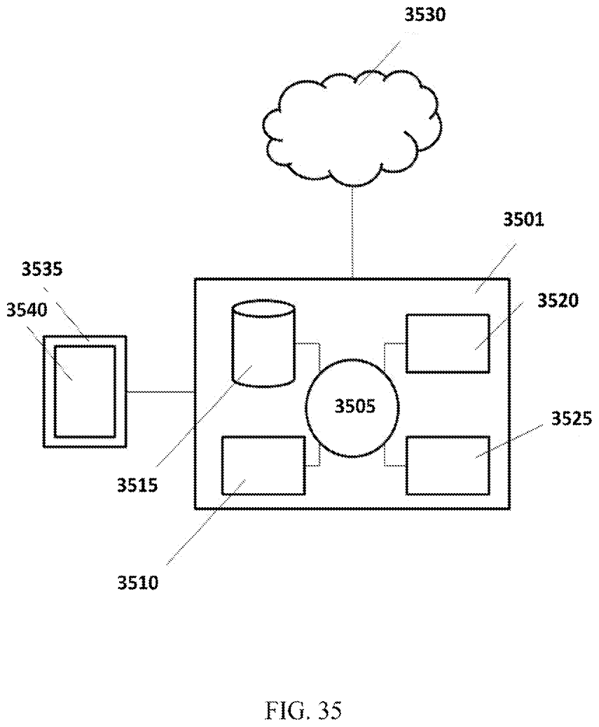

The present disclosure provides computer control systems that are programmed to implement methods of the disclosure. FIG. 35 shows a computer system 3501 that is programmed or otherwise configured to implement methods and systems of the present disclosure, such as performing nucleic acid sequence and sequence analysis.

The computer system 3501 includes a central processing unit (CPU, also “processor” and “computer processor” herein) 3505, which may be a single core or multi core processor, or a plurality of processors for parallel processing. The computer system 3501 also includes memory or memory location 3510 (e.g., random-access memory, read-only memory, flash memory), electronic storage unit 3515 (e.g., hard disk), communication interface 3520 (e.g., network adapter) for communicating with one or more other systems, and peripheral devices 3525, such as cache, other memory, data storage and/or electronic display adapters. The memory 3510, storage unit 3515, interface 3520 and peripheral devices 3525 are in communication with the CPU 3505 through a communication bus (solid lines), such as a motherboard. The storage unit 3515 may be a data storage unit (or data repository) for storing data. The computer system 3501 may be operatively coupled to a computer network (“network”) 3530 with the aid of the communication interface 3520. The network 3530 may be the Internet, an internet and/or extranet, or an intranet and/or extranet that is in communication with the Internet. The network 3530 may be a telecommunication and/or data network. The network 3530 may include one or more computer servers, which may enable distributed computing, such as cloud computing. The network 3530, with the aid of the computer system 3501, may implement a peer-to-peer network, which may enable devices coupled to the computer system 3501 to behave as a client or a server.

The CPU 3505 may execute a sequence of machine-readable instructions, which may be embodied in a program or software. The instructions may be stored in a memory location, such as the memory 3510. The instructions may be directed to the CPU 3505, which may subsequently program or otherwise configure the CPU 3505 to implement methods of the present disclosure. Examples of operations performed by the CPU 3505 may include fetch, decode, execute, and writeback.

The CPU 3505 may be part of a circuit, such as an integrated circuit. One or more other components of the system 3501 may be included in the circuit. The circuit may be an application specific integrated circuit (ASIC).

The storage unit 3515 may store files, such as drivers, libraries and saved programs. The storage unit 3515 may store user data, e.g., user preferences and user programs. The computer system 3501 may include one or more additional data storage units that are external to the computer system 3501, such as located on a remote server that is in communication with the computer system 3501 through an intranet or the Internet.

The computer system 3501 may communicate with one or more remote computer systems through the network 3530. For instance, the computer system 3501 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 3501 via the network 3530.

Methods as described herein may be implemented by way of machine (e.g., computer processor) executable code stored on an electronic storage location of the computer system 3501, such as, for example, on the memory 3510 or electronic storage unit 3515. The machine executable or machine readable code may be provided in the form of software. During use, the code may be executed by the processor 3505. The code may be retrieved from the storage unit 3515 and stored on the memory 3510 for ready access by the processor 3505. In some situations, the electronic storage unit 3515 may be precluded, and machine-executable instructions are stored on memory 3510.

The code may be pre-compiled and configured for use with a machine having a processer adapted to execute the code or may be compiled during runtime. The code may be supplied in a programming language that may be selected to enable the code to execute in a pre-compiled or as-compiled fashion.

Aspects of the systems and methods provided herein, such as the computer system 3501, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 3501 may include or be in communication with an electronic display 3535 that comprises a user interface (UI) 3540 for providing, for example, imaging results or results of nucleic acid sequencing (e.g., sequence reads, consensus sequences, etc.). Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 3505. The algorithm can, for example, implement methods of the present disclosure.

### VI. Sample Preparation

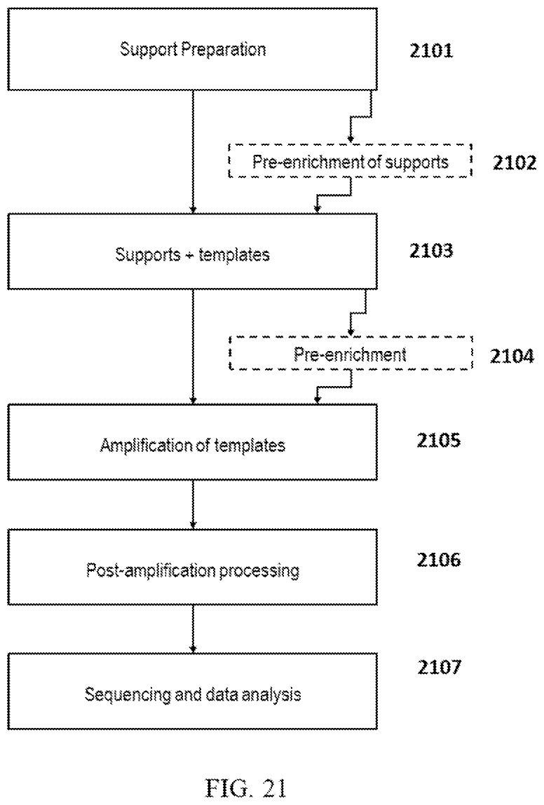

FIG. 21 illustrates example workflows for sample preparation and analysis, according to systems, compositions, and methods of the present disclosure.

Supports may be used preparing a sample and/or during analysis of the sample. Such supports may be prepared (2101) according to the systems, compositions, and methods described herein. For example, a population of supports may be prepared to comprise a plurality of unique support species, as described with respect to, for example, FIGS. 3A-3B, FIG. 4, where each unique support species comprises a primer sequence unique to said support species. When attaching template nucleic acid molecules to supports of said support species, only a template nucleic acid molecule comprising an adapter sequence compatible with (e.g., at least partially complementary to) a given primer sequence may be capable of attaching to a given support comprising the given primer sequence. In another example, a population of supports may be prepared, such that each unique support species comprises a plurality of primer sequences (e.g., a pair of primer sequences) unique to said support species (e.g., as described with respect to, for example, FIGS. 3C-3G). Example methods for generation of such supports are described with respect to, for example, FIGS. 2, 9, and 10A-10B.

In some embodiments, the systems and methods disclosed herein can include supports that comprise two, three, four, five, six, seven, eight, nine, ten unique support species. Each unique support species can comprise a unique primer sequence that allows selective interactions between the respective support species with an intended binding partner (e.g., a complementary nucleic acid sequence within an adapter region of a sample nucleic acid or an extension primer sequence which can subsequently bind to a complementary nucleic acid sequence within an adapter region of a sample nucleic acid). In some embodiments, the systems and methods disclosed herein can include supports that comprise more than ten unique support species. Each unique support species can comprise a unique primer sequence that allows selective interactions between the respective support species with an intended binding partner (e.g., a complementary nucleic acid sequence within an adapter region of a sample nucleic acid or an extension primer sequence which can subsequently bind to a complementary nucleic acid sequence within an adapter region of a sample nucleic acid).

Optionally, supports may be pre-enriched (2102), wherein a support comprising a distinct primer sequence, or an extended primer sequence, is isolated from a mixture comprising support(s) that do not have the distinct primer sequence, or the extended primer sequence, such as described with respect to, for example, FIGS. 2, 10A-10B, and 11. Alternatively, a support population may be provided to comprise substantially uniform supports, where each support comprises an identical surface primer molecule immobilized thereto.

Subsequent to support preparation, a library of template molecules may be attached to the supports (2103). In some instances, template molecules may be prepared by ligating desired adapter sequence(s) to sample molecules to generate template molecules comprising the desired adapter sequence(s), such as described with respect to, for example, FIG. 8. A template molecule may be coupled to a support via any methods described herein, such as by hybridization of the template molecule to a surface primer of a support and/or subsequent extension of the surface primer thereof. Example methods for attaching template molecules to supports are described with respect to, for example, FIGS. 1-4, 8, 12A-12B, and 20A-20B.

Optionally, supports may be pre-enriched (2104), wherein a support comprising a template nucleic acid molecule, or a sequence corresponding to a template nucleic acid sequence, is isolated from a mixture comprising support(s) that do not have attached template nucleic acid molecules, or template nucleic acid sequences, such as described with respect to, for example, FIGS. 1, 12A-12B, 20A-20B and 22A-22C.

Subsequent to attachment of the template nucleic acid molecule to the support, the template nucleic acid molecule may be subjected to amplification reactions (2105) to generate a plurality of amplification products immobilized to the support. For example, such amplification reactions may comprise performing polymerase chain reaction (PCR) or any other amplification methods. Example methods of on-support amplification are described with respect to, for example, FIGS. 3C-3G and FIG. 4. Amplification may comprise performing isothermal amplification (e.g., recombinase polymerase amplification (RPA)), bridge amplification, wildfire (e.g., template walking) amplification, etc.

Subsequent to amplification, the supports (e.g., comprising the template nucleic acid molecules) may be subjected to post-amplification processing (2106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described with respect to, for example, FIGS. 18A-18E, and 19A-19B. For example, an on-substrate enrichment procedure (e.g., as shown in FIGS. 19A-19B) may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed (e.g., as shown in FIGS. 18A-18E) to selectively remove unamplified surface primers (on the support(s)), such that a resulting positive support retains the template nucleic acid molecule, and a resulting negative support is stripped of the unamplified surface primers. Subsequently, the template nucleic acid molecule(s) on the positive supports may be used to enrich for the positive supports, e.g., by capturing the template nucleic acid molecules.

Subsequent to post-amplification processing (2106), the template nucleic acid molecules may be subject to sequencing and/or data analysis. For example, the template nucleic acid molecules may be sequenced while attached to the support. Alternatively, the template nucleic acid molecules may be free of the support when sequenced and/or analyzed. In some instances, the template nucleic acid molecules may be sequenced while attached to the support which is attached to a substrate surface.

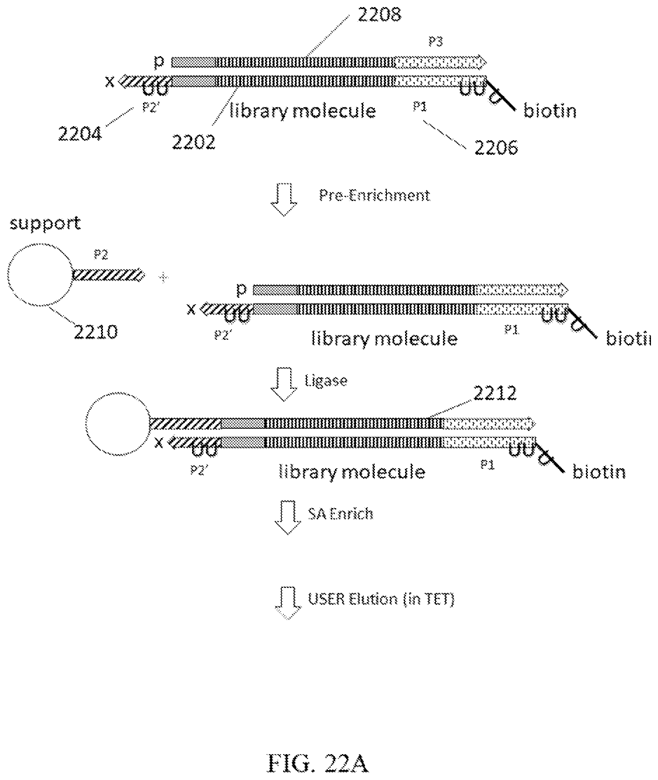

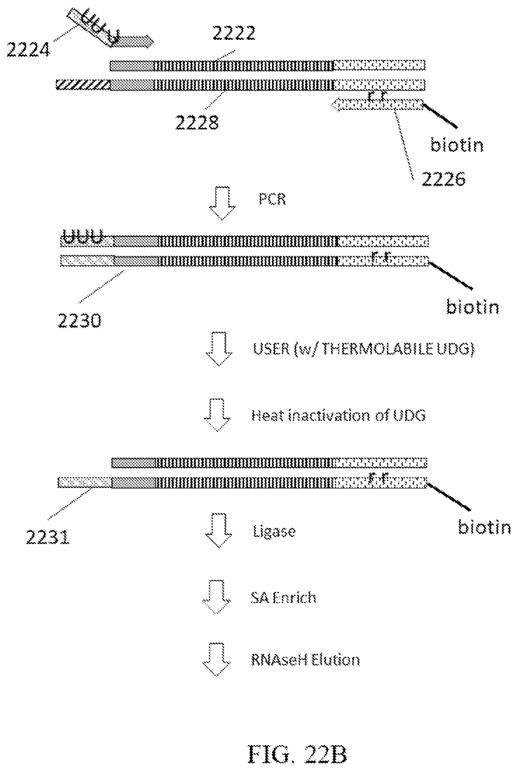

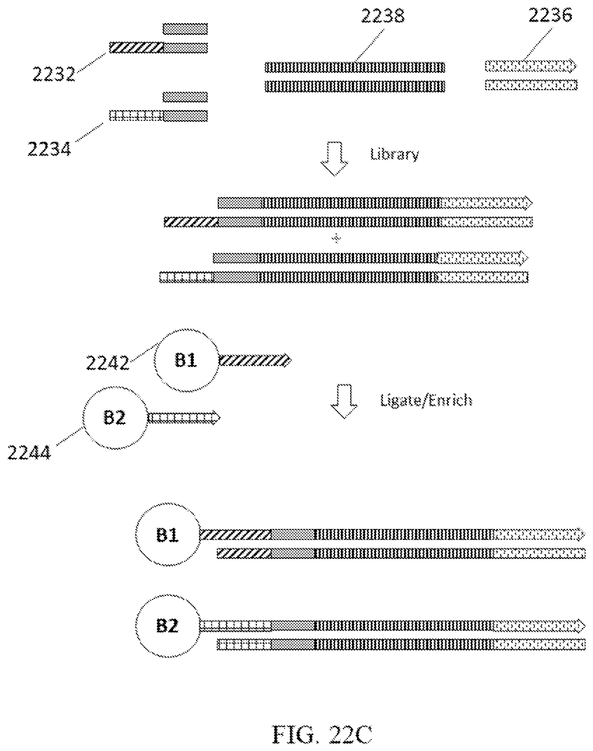

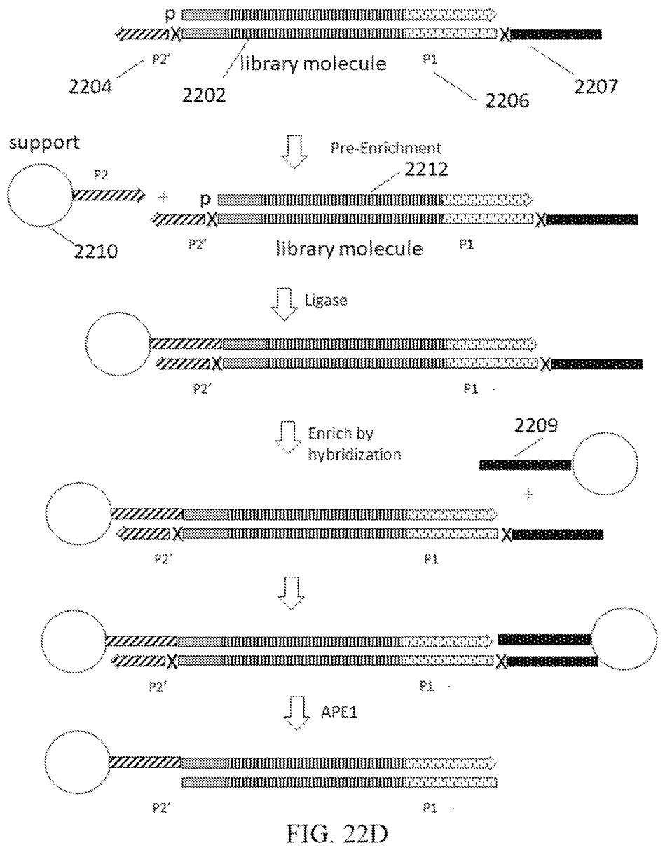

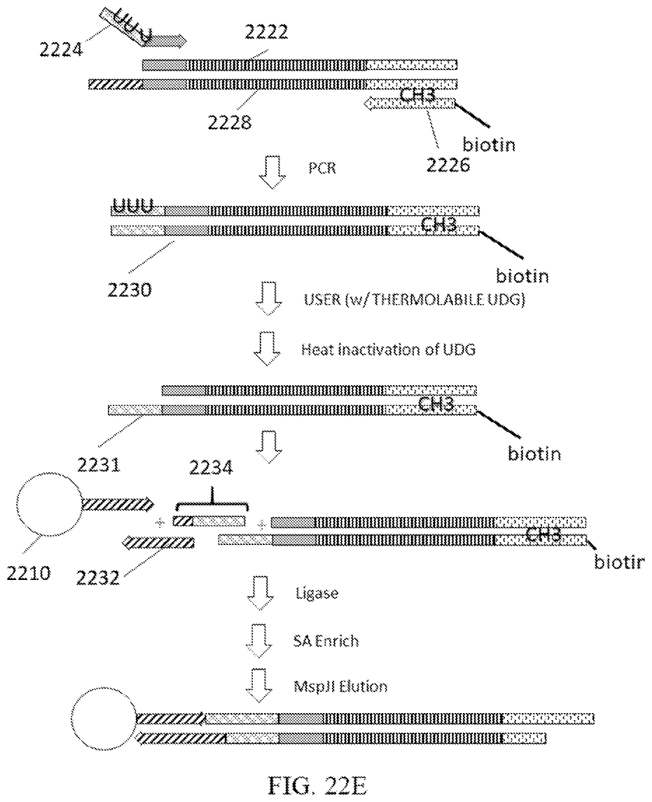

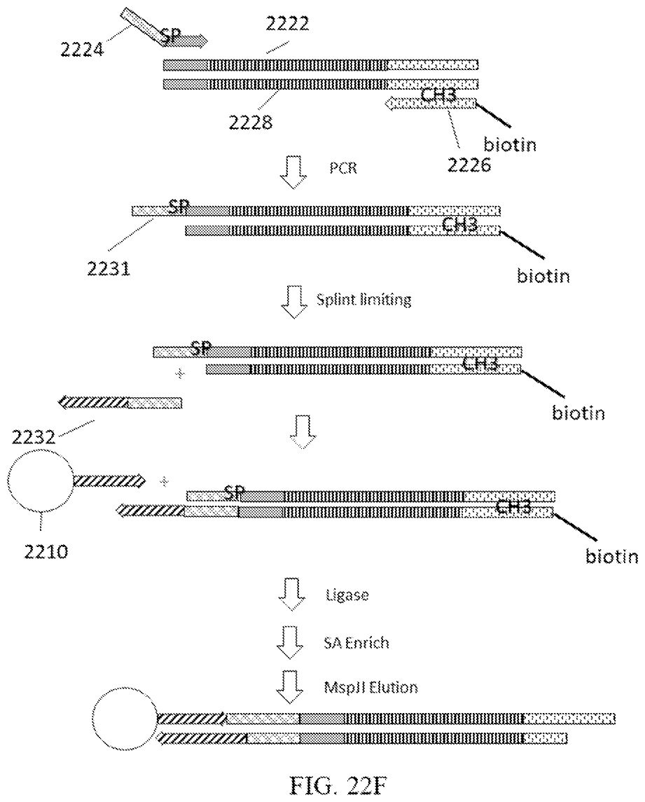

FIGS. 22A-22D show examples of pre-enrichment (e.g., 2104) schemes that can be used in combination with one or more of the other methods described herein. As illustrated in FIG. 21, pre-enrichment (2104) can occur prior to amplification and sequencing. FIG. 22A illustrates an example where adaptors including one or more cleavable moieties (e.g., a uracil or a ribonucleotide) are used. FIG. 22B illustrates an example where adapters comprising one or more cleavable moieties of different types (e.g., a uracil and a ribonucleic acid) are used. FIG. 22C illustrates an example where different support species having surface primer sequences complementary to different adaptor sequences can be used to bind to template nucleic acid molecules to decrease the possibility of different template nucleic acid molecules binding to the same support. FIG. 22D illustrates an alternative pre-enrichment process where pre-enrichment occurs via hybridization of complementary sequences.

As shown in FIG. 22A, in some embodiments, a double stranded template nucleic acid molecule (e.g., of a library of template nucleic acid molecules) comprises, ligated at each end, an adapter containing one or more cleavable moieties (e.g. uracils). The methods for pre-enrichment described herein may comprise generating such double stranded template nucleic acid molecules. The double stranded template nucleic acid molecule may comprise a template sequence. The double stranded template nucleic acid molecule may comprise different sequences at the 5′ and 3′ ends. For example, the 5′ and 3′ ends of a first strand (e.g., strand 2202) of the double stranded template nucleic acid molecule may comprise different overhang sequences. In this example, each of the 5′ and 3′ ends comprises an adaptor with a plurality of uracils. In some embodiments, one adapter containing one or more cleavable moieties (e.g., uracils) may be on a first strand (e.g., a top strand) and another adapter containing one or more cleavable moieties (e.g., uracils) may be on a second strand (e.g., a bottom strand) of the double stranded template nucleic acid molecule (e.g., both adapters may be on 5′ ends or both may be on 3′ ends). On the 5′ end of the first strand 2202, sequence P1 (e.g., sequence 2206) comprises an overhang comprising one or more uracils, where the overhang is connected to a biotin molecule (e.g., the 5′ end of first strand is biotinylated). On the 3′ end of the first strand 2202, sequence P2′ (e.g., sequence 2204) comprises an overhang sequence including one or more cleavable moieties (e.g., a uracil or a ribonucleotide). In some embodiments, as described for example below, other cleavable sequences (comprising one or more cleavable moieties) can be used at either or both of the 3′ and 5′ ends of the first strand 2202 (e.g., in place of the pluralities of uracils in sequence 2204 and sequence 2206). The sequence P2′ is at least partially complementary to a sequence P2 bound to support 2210, such as a bead. In some embodiments, the biotin molecule instead of being connected to the first strand at the 5′ end, may be connected to a second strand of the double stranded template nucleic acid molecule (e.g., at the 3′ end via sequence P3, modified with a cleavable sequence). Optionally, a polymerase (e.g., Taq polymerase or another polymerase with 5′-3′ exonuclease activity) and reaction reagents (e.g., nucleotide bases) may be used to process the double stranded template nucleic acid molecule, e.g., to facilitate hybridization or ligation of the double stranded template nucleic acid molecule to the support 2210. For example, the polymerase may cleave off any remaining uncleaved moieties (e.g., ribonucleotides, uracil), generating a phosphorylated end (e.g., phosphorylated 5′ end) of the template nucleic acid molecules. After the template nucleic acid molecule hybridizes to support 2210, a ligase is used to repair the nick such that the second strand 2212 is covalently attached to support 2210. An advantage of this uracil-based protocol is that, after enrichment, the nucleic acid molecule fragment (e.g., second strand 2212) bound to support 2210, such as a bead, is the original fragment. Thus, for example, one fewer PCR amplification step is required, thus reducing the potential for introducing sequencing errors. In addition to one or more cleavable moieties, sequence 2206 can further comprises an affinity tag such as a biotin. The affinity tag may comprise any appropriate capture moiety, configured for capture by a capturing moiety, as described elsewhere herein. After strand 2212 is covalently linked to support 2210, magnetic beads each comprising a capturing moiety (e.g., streptavidin) may be introduced to a reaction mixture containing numerous supports (some of which are covalently linked with template nucleic acid molecules and some of which are not). Supports covalently linked to a template nucleic acid molecule may have the affinity tag (e.g., a biotin tag) that binds to the capturing moiety (e.g., streptavidin), and therefore can be selectively pulled down or otherwise isolated using the magnetic beads comprising the capturing moiety, such as via applying a magnetic field. As a result, the supports comprising the respective template nucleic acid molecules coupled thereto (e.g., covalently linked thereto) may be isolated and enriched from the other supports that do not have a template nucleic acid molecule attached. USER (Uracil-Specific Excision Reagent) enzymes can then be added to the reaction mixture to cleave off the uracils that are 3′ to the biotin tag before the bead-template nucleic acid complexes are further processed (e.g., amplified and sequenced).

As shown in FIG. 22B, in some embodiments, template nucleic acid molecules are already attached to adapters that may not be compatible with a pre-enrichment scheme as disclosed herein. A template nucleic acid molecule may be generated from a duplex template molecule using a combination of primers comprising different cleavable moieties (e.g., a uracil-containing primer and a ribonucleic acid-containing primer). For example, a first primer (e.g., primer 2224) that is complementary to a first strand 2222 of the duplex template molecule comprises a plurality of uracils (e.g., a uracil-based cleavage point). A second primer (e.g., primer 2226) that is complementary to second strand 2228 comprises a plurality of ribonucleic acid bases. A PCR amplification operation is performed to generate a double-stranded intermediate template nucleic acid molecule 2230. An enzyme (e.g., USER with a thermolabile UDG: uracil DNA glycosylase) may then be used to cleave the one or more cleavable moieties (e.g., a uracil) in the sequence corresponding to primer 2224. After the enzyme treatment, a 3′ overhang 2231 is generated which can then bind to a complementary sequence on a support, as described elsewhere herein. Optionally, a polymerase (e.g., Taq polymerase) and reaction reagents (e.g., nucleotide bases) may be used to process the double stranded template nucleic acid molecule, e.g., to facilitate hybridization or downstream ligation of the double stranded template nucleic acid molecule to the support 2210. Similarly, a ligase can be added to covalently attach the first strand 2222 to the support. The reaction mixture can then be processed as described above with respect to FIG. 22A.

FIG. 22C illustrates that, in some embodiments, double stranded template nucleic acid molecules (e.g., from a library of template nucleic acid molecules) can be ligated to supports (e.g., beads) having different surface primers attached thereon (e.g., a population of different bead species with at least 2 different surface primer sequences, at least 3 different surface primer sequences, at least 5 different surface primer sequences, etc.). FIG. 22C shows annealing of template nucleic acid molecules to two bead species with two different surface primer sequences (e.g., bead species 2242 and 2244). Similar to the protocol presented in FIG. 22A, double stranded template molecules 2238 may be ligated to a different adapter at each end. For example, provided a population of initial template molecules 2238, a first adapter 2236 (e.g., barcoded adaptor) can be added at one end and a mixture of two different types second adapters (e.g., adapters 2232 and 2234) can be added and ligated to the other end of the template molecules 2238. The second adapters may comprise 5′ bead adapter primers. The ligation of the second adapters may occur prior to, during, or after the ligation of the first adapters. The mixture of second adapters may comprise adapters with different sequences. The mixture of second adapters may be in equimolar concentrations of the adapters with different sequences. Alternatively, the mixture of second adapters may be in any other concentrations (e.g., engineered for the subsequent operations). Alternatively or in addition, a sample comprising the template molecules 2238 are divided in two portions and the two different second adapters (2232 and 2234) can be ligated to the template molecules in separate reactions (e.g., in separate reaction spaces). In some embodiments, one or more the adapters (e.g., 2232, 2234, and 2236) can include a barcode sequence. The resulting template nucleic acid molecules can selectively bind to supports (e.g., beads) comprising sequences that are complementary to the overhang sequences in adapter 2232 or 2234. This permits the ligation of different support species (e.g., bead 2242 and bead 2244) to different populations of template molecules in the library, thus permitting different types of downstream analysis. Though not illustrated in FIG. 22C, enrichment schemes as disclosed in FIGS. 22A and 22B can be used for the template nucleic acid molecules generated in accordance with FIG. 22C. For example, one or more uracils and an affinity tag (e.g., streptavidin) can be included in first adapter 2236, thus allowing template loaded beads to be pulled down by capturing moieties (e.g., streptavidin bound magnetic beads). Similarly, one or more uracils can be included in the bottom strand of the second adapters 2232, 2234, as depicted in FIG. 22A.

FIG. 22D schematically shows, similar to FIG. 22A, an additional pre-enrichment workflow. In some embodiments, a double stranded template nucleic acid molecule comprises, ligated at each end, an adapter containing one or more cleavable moieties (e.g., an excisable nucleotide such as uracil, a dSpacer, a C3 spacer, or other abasic site, a methylated nucleotide, etc., illustrated as “X”). The methods for pre-enrichment described herein may comprise generating such double stranded template nucleic acid molecules. The double stranded template nucleic acid molecule may comprise a template sequence. In such an example, the 5′ and 3′ ends of a first strand (e.g., strand 2202) of the double stranded template nucleic acid molecule may comprise different overhang sequences. For example, each of the 5′ and 3′ ends may comprise an adaptor with a single or plurality of cleavable moieties (e.g., C3 spacer, methylated nucleotide). On the 5′ end of the first strand 2202, sequence P1 (e.g., sequence 2206) comprises an overhang comprising a cleavable moiety (such as a C3 spacer, e.g., “X”), and an overhang sequence 2207, which may be used in downstream capture. On the 3′ end of the first strand 2202, sequence P2′ (e.g., sequence 2204) comprises an overhang sequence including one or more cleavable moieties (e.g., a C3 spacer, e.g., “X”). In some embodiments, as described for example below, other cleavable sequences (comprising one or more cleavable moieties) can be used at either or both of the 3′ and 5′ ends of the first strand 2202 (e.g., in place of the C3 spacers in sequence 2204 and sequence 2206). Similar to FIG. 22A, the sequence P2′ is at least partially complementary to a sequence P2 bound to support 2210, such as a bead. Optionally, a polymerase (e.g., Taq polymerase) and reaction reagents (e.g., nucleotide bases) may be used to process the double stranded template nucleic acid molecule, e.g., to facilitate hybridization of the double stranded template nucleic acid molecule to the support 2210. After the template nucleic acid molecule hybridizes to support 2210, a ligase is used to repair the nick such that the second strand 2212 is covalently attached to support 2210.

In addition to one or more cleavable moieties, sequence 2206 can comprise an overhang sequence 2207 or any appropriate capture moiety. In some examples, the overhang sequence is configured for capture by a capturing moiety, e.g., a capture oligonucleotide. After strand 2212 is covalently linked to support 2210, capture beads (e.g., magnetic beads) each comprising a capture oligonucleotide 2209 may be introduced to a reaction mixture containing numerous supports (some of which are covalently linked with template nucleic acid molecules and some of which are not). In some instances, at least a portion of the capture oligonucleotide 2209 is complementary to the overhang sequence 2207 of strand 2202. Supports that are covalently linked to a template nucleic acid molecule may have the overhang sequence 2207 that binds to the capture oligonucleotide 2209, and therefore can be selectively pulled down or otherwise isolated using the capture beads (e.g., magnetic beads) comprising the capturing moiety, such as via applying a magnetic field. As a result, the supports comprising the respective template nucleic acid molecules coupled thereto (e.g., covalently linked thereto) may be isolated and enriched from the other supports that do not have a template nucleic acid molecule attached. An enzyme, such as an endonuclease, e.g., APE1, MspJI, etc., can then be added to the reaction mixture to cleave off the C3 spacers (or methylated nucleotides) that are 3′ to the overhang sequence 2207 before the support-template nucleic acid complexes are further processed (e.g., amplified and sequenced). See also, Example 12.

As described herein, the cleavable moiety may comprise any useful cleavable or excisable moiety. For example, the cleavable moiety may comprise a uracil, a ribonucleotide, or other modified nucleotide that is excisable or cleavable using an enzyme (e.g., UDG, RNAse, endonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using a stimulus, e.g., photo-stimulus, chemical stimulus, thermal stimulus, etc., as described elsewhere herein.

It will be appreciated that combinations of cleavable moieties may be attached (e.g., via ligation or amplification of adapter molecules) to the template nucleic acid molecules. For example, referring to FIGS. 22A-22D, the first strand 2202 may comprise the same or different cleavable moieties at the 5′ end and the 3′ end. In some examples, the first strand comprises one or more cleavable uracil moieties at an end (5′ or 3′) and one or more ribonucleotides at the opposite (3′ or 5′) end. In another example, the first strand comprises one or more C3 spacer moieties at an end, and also a C3 spacer at the opposite end. It will be appreciated that any combination of cleavable moieties (e.g., uracils, ribonucleotides, spacers, abasic sites, enzyme-specific modified nucleotides, such as methylated nucleotides, etc.) may be incorporated at either or both the 5′ end and 3′ end of the template nucleic acid molecule. Similarly, in instances where the template nucleic acid molecule is double stranded, the cleavable moieties may be disposed on different strands. For example, one strand may comprise a cleavable moiety at the 5′ end and the other strand may comprise a cleavable moiety at the 5′ end. Alternatively, one strand may comprise a cleavable moiety at the 3′ end and the other strand may comprise a cleavable moiety at the 3′ end. As different moiety types and locations are possible, the methods described herein may also provide for orthogonal cleavage mechanisms, such that one moiety at one end of a template nucleic acid molecule may be cleavable but another cleavable moiety on the other end of the template nucleic acid molecule may not be cleavable by the same mechanism. Such orthogonal cleavage mechanisms may be beneficial in multiple processing operations; for example, the template molecule may comprise different cleavable moieties (e.g., a first moiety and a second moiety) at each end. Accordingly, attachment of template molecules to supports may be facilitated by cleavage or excision of the first moiety that does not affect the second moiety. Subsequent ligation and pre-enrichment may be performed, and elution of the supports may be facilitated by cleavage of the second moiety. In some embodiments, following elution of the supports, amplification of the template molecules is performed on the support, as is described elsewhere herein. In such instances, it may be useful to have a different cleavable moiety, e.g., on the template nucleic acid molecule, e.g., at or proximal to the support, or on the primer coupled to the support.

Referring again to FIG. 22D, an overhang sequence of an adaptor on the template nucleic acid molecule may be used for purification or pre-enrichment via capture using a bead with a complementary oligonucleotide. The overhang sequence of the adaptor can be any useful length, e.g., 1, 2, 3, 4, 5, 6, 7, 8, 9, 10, 20, 30, 40, 50, 60, 70, 80, 90, 100 or greater base pairs. The overhang sequence may be at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100 or greater base pairs. Alternatively, the overhang sequence may be at most 100, at most 90, at most 80, at most 70, at most 60, at most 50, at most 40, at most 30, at most 20, at most 10, at most 9, at most 8, at most 7, at most 6, at most 5, at most 4, at most 3, at most 2, or at most 1 base pair in length. In some instances, the complementary oligonucleotide (e.g., coupled to bead) may comprise a sequence that is complementary to the entire overhang sequence of the template nucleic acid molecule, or a portion thereof.

In some embodiments, the template nucleic acid molecules (e.g., from a library of template nucleic acid molecules) attach indirectly to the supports, e.g., via intermediary molecules such as splint and/or bridge oligonucleotides. As shown in FIGS. 22E-22F, attachment of the template nucleic acid molecules may be facilitated by a splint oligonucleotide 2232 and optionally, a bridge molecule 2234. For example, FIG. 22E shows an example workflow, similar to FIG. 22B, in which a template nucleic acid molecule is generated from a duplex template molecule using a combination of primers comprising different cleavable moieties (e.g., a uracil-containing primer 2224 and a methylated nucleotide-containing primer 2226). Following amplification, the template nucleic acid molecules may be contacted with an enzyme (e.g., USER with a thermolabile UDG) to cleave one or more cleavable moieties (e.g., uracil) of the template nucleic acid molecule, thus generating a 3′ overhang 2231. The overhang 2231 may not be complementary to a sequence on a support 2210. Attachment of the overhang 2231 may be facilitated by a bridge oligonucleotide 2234 and a splint oligonucleotide 2232. The bridge oligonucleotide 2234 may comprise a sequence complementary to at least a portion of the overhang 2231 and another sequence that is complementary to at least a portion of the splint oligonucleotide 2232. The splint oligonucleotide 2232 may comprise a sequence complementary to a sequence on the support 2210. Accordingly, hybridization of the template nucleic acid molecule to the bridge oligonucleotide 2234 and the splint oligonucleotide 2232 may facilitate attachment of the template nucleic acid molecule to the support 2210. Following hybridization, a ligase and optionally a polymerase may be added to covalently attach the first strand 2222 to the support 2210. Subsequent processing (e.g., enrichment, treatment with an enzyme to cleave one or more cleavable moieties (e.g., MspJI to cleave methylated nucleotides) may be performed, as is described elsewhere herein.

FIG. 22F shows an alternative scheme for attachment of a template nucleic acid molecule to a support using a splint oligonucleotide (also referred to herein as “splint molecule”). Similar to FIG. 22B and FIG. 22E, a template nucleic acid molecule is generated from a duplex template sequence using a combination of primers comprising different cleavable moieties (e.g., a spacer-containing primer 2224 and a methylated nucleotide-containing primer 2226). In some instances, one of the primers 2224 may comprise a moiety that inhibits amplification (e.g., a spacer moiety). The amplification-inhibiting moiety may be the same as the cleavable moiety. Following amplification, the template nucleic acid molecule may comprise an overhang sequence 2231. A splint molecule 2232 may be contacted with the template nucleic acid molecule. The splint molecule 2232 may comprise a sequence complementary to the overhang 2231 as well as a sequence complementary to a sequence of a support 2210. Accordingly, hybridization of the template nucleic acid molecule to the splint oligonucleotide 2232 may facilitate attachment of the template nucleic acid molecule to the support 2210. Following hybridization of the splint oligonucleotide 2232 to the sequence of the support 2210, a ligase and optionally a polymerase may be added to covalently attach the first strand 2222 to the support 2210. Subsequent processing, e.g., enrichment, treatment with an enzyme to cleave one or more cleavable moieties (e.g., MspJI to cleave methylated nucleotides) may be performed, as is described elsewhere herein.

While FIGS. 22E-22F illustrate the splint and bridge molecules provided as separate items, it will be appreciated that the splint molecule and/or bridge molecule may be provided as a pre-annealed complex. For instance, referring the FIG. 22E, the bridge molecule 2234 may be provided pre-annealed to the splint molecule 2232. Alternatively, or in addition to, the splint molecule 2232 may be provided pre-annealed to the support 2210. Similarly, referring to FIG. 22F, the splint molecule 2232 may be provided as pre-annealed to the support 2210.

As shown in FIGS. 22E-22F, in some instances, hybridization and attachment of the template nucleic acid molecule to the support may only occur if a splint (and, in some cases, a bridge) is provided. Accordingly, the ratio of support-to-template nucleic acid molecule attachment may be controlled by controlling the amount of splint (and bridge) molecules that is provided. For example, where an excess of template nucleic acid molecules is provided, any useful support-to-template nucleic acid molecule ratio may be achieved by controlling the amount of splint (and bridge) molecules that is provided. As the splint oligonucleotide (and bridge) is the rate-limiting reagent, using such a splint-mediated process may obviate the need to quantify or quantitate the number of ligatable template nucleic acid molecules (e.g., UDG-treated template nucleic acid molecules that have 3′ ligatable ends). Since these ligatable molecules may be challenging to quantitate, using the splint-mediated attachment approach can overcome this challenge, since the attachment ratio of the template nucleic acid molecule to the support is controlled by the splint (and bridge) concentration. See also, Example 15. In other instances, the template nucleic acid molecules may be quantitated (e.g., using qPCR) and the attachment ratio to the supports may be controlled using the splint and/or bridge molecule concentration.

In some examples, the ratio of the splint (and/or bridge) molecule-to-template nucleic acid molecule ratio may be about 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100 or less. In other examples, it may be useful to capture as many of the template nucleic acid molecules on the supports, and accordingly, the ratio of the splint (and/or bridge) molecule-to-template nucleic acid molecule ratio may be greater than 1, e.g., about 2 times (×), about 3×, about 4×, about 5×, about 6×, about 7×, about 8×, about 9×, about 10×, or greater. It will be appreciated that a range of concentrations of the splint, bridge, template nucleic acid molecules, and supports may be varied according to a designated attachment ratio of the template nucleic acid molecule to the support. As described above, in some instances, the bead is provided at a lower concentration than the concentration of the template (and/or splint and bridge molecules), which may facilitate generation of monoclonal support populations.

The bridge or splint molecule may comprise any useful number of nucleotide bases, e.g., between about 10-100 nucleotide bases. The bridge or splint molecule, or both, may comprise at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200 or more nucleotide bases. The bridge or splint molecule, or both, may comprise at most about 200, at most 100, at most 90, at most 80, at most 70, at most 60, at most 50, at most 40, at most 30, at most 20, at most 10, at most 9, at most 8, at most 7, at most 6, at most 5, at most 4, at most 3, at most 2, or at most 1 nucleotide base. It will be appreciated that the bridge or splint molecule, or both, may comprise a range of nucleotide base lengths, e.g., about 30 bases. The bridge molecule may have the same length (e.g., number of bases) as the splint molecule, or the bridge molecule may have a different length than the splint molecule. The bridge molecule and the splint molecule may comprise complementary sequences to facilitate hybridization; such complementary sequences may be any useful length, e.g., about 1, about 2, about 3, about 4, about 5, about 6, about 7, about 8, about 9, about 10, about 15, about 20, about 30, about 40, about 50 or more nucleotides in length. The complementary sequences may be at least 1, at least 2, at least 3, at least 4, at least 5, at least 6, at least 7, at least 8, at least 9, at least 10, at least 20, at least 30, at least 40, at least 50 or more nucleotides in length. Alternatively, the complementary sequences may be at most 50, at most 40, at most 30, at most 20, at most 10, at most 9, at most 8, at most 7, at most 6, at most 5, at most 4, at most 3, at most 2 or fewer nucleotides in length.

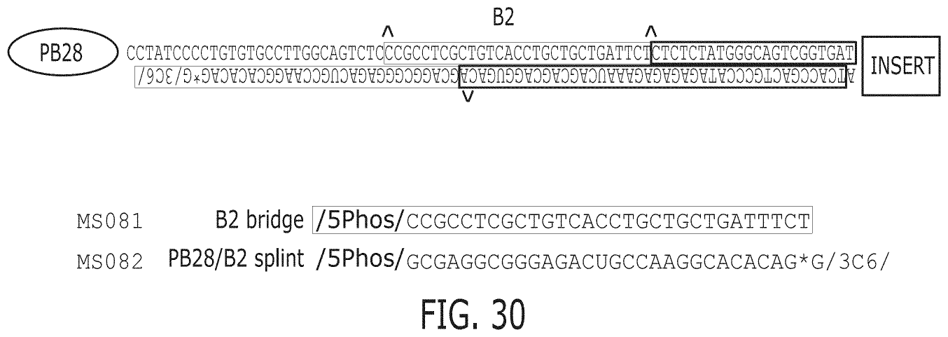

The bridge and splint molecules may comprise any useful nucleotide sequence. For instance, it may be useful to have a predefined GC content to facilitate annealing or hybridization. Accordingly, the bridge and splint molecule composition and sequence may be altered according to any useful application (e.g., for attachment of the template nucleic acid molecule to a support). Similarly, the nucleotide sequence of the bridge and splint molecule may be adjusted to achieve a specified annealing and/or melting temperature. An example of a bridge sequence (“B2 bridge”) and a splint molecule (“B2 splint”) for attachment of a template nucleic acid molecule to a support (e.g., bead, “PB28”) is shown in FIG. 30.

The methods for pre-enrichment described herein may additionally comprise generating the template nucleic acid molecules, e.g., prior to attachment of the template nucleic acid molecules to a support. The template nucleic acid molecules may be single stranded, double stranded, or partially double stranded. As illustrated in FIGS. 22A-22F, the double stranded template nucleic acid molecules may be generated by attaching one or more adapters to an initial template sequence (e.g., 2238), e.g., via ligation or amplification. In some instances, the one or more adapters may be ligated to the initial template sequence using a splint ligation method. Examples of splint ligation methods can be found in Single Reaction Single-stranded Library (SRSLY) (see, e.g., C. J. Troll et al. BMC Genomics, 20, 1023 (2019) or Splinted Ligation Adapter Tagging (SPLAT) approach (see, e.g., A. Raine et al. Nucl. Acids Research, vol 45, no. 6 (2017), each of which is hereby incorporated by reference herein in its entirety). In such instances, the adapters may comprise a sequence that is complementary to a strand of the initial template sequence. In some examples, the adapters may comprise additional functional sequences (e.g., primer sequences) or moieties (e.g., blocking moieties).

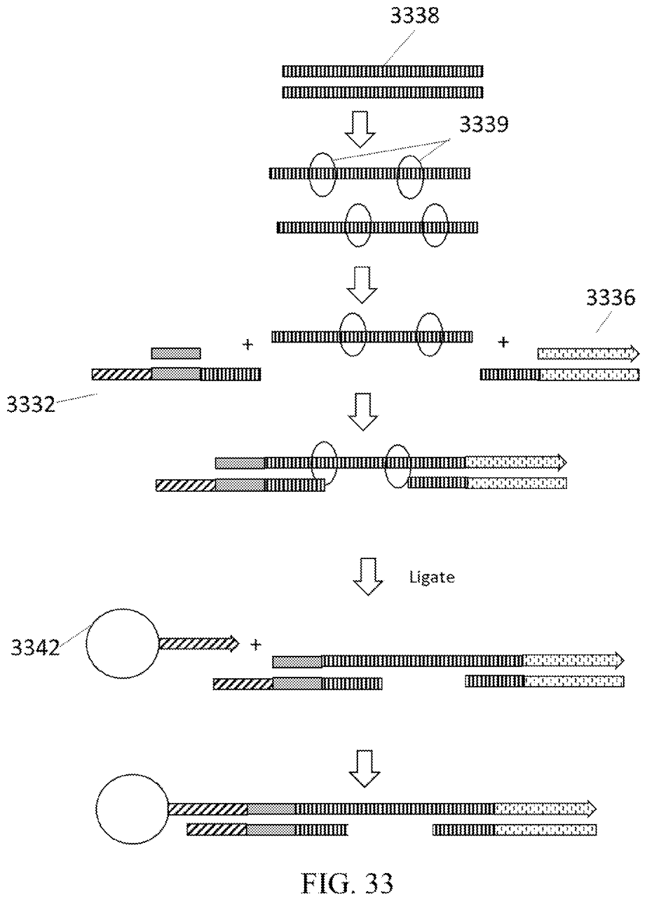

FIG. 33 shows an example workflow of generating a partially double stranded template nucleic acid molecule, which can subsequently be attached to a support (e.g., bead) and further processed (e.g., pre-enriched, sequenced, etc.). A template sequence (e.g., double stranded template sequence) 3338 may be subjected to conditions sufficient to generate single-stranded template sequences. In some instances, the conditions comprise using heat or a chemical denaturant to generate the single-stranded template sequences. Alternatively or in addition to, a protein such as a single-stranded nucleic acid binding protein 3339 may be provided to generate or maintain the single-strandedness of the template sequences 3338. The single-stranded template sequences may be ligated to one or more adapters at each end. For example, a first adapter 3336 may be ligated at one end and a second adapter 3332 may be ligated at the other end. In some instances, the first adapter 3336 and/or the second adapter 3332 are partially double-stranded. The first adapter 3336 and/or second adapter 3332 may comprise a single-stranded overhang sequence, which may be complementary and may hybridize to a portion of the single-stranded template sequence. The overhang sequence may comprise, for example, a random N-mer or a targeted primer sequence. In some instances, the overhang sequence of the first adapter 3336 may be disposed at the 5′ terminus, and the overhang sequence of the second adapter 3332 may be disposed at the 3′ terminus, such that the polarity or directionality of the initial template sequence is maintained in subsequent processing operations (e.g., amplification, pre-enrichment, sequencing, etc.). For instance, the first adapter 3336 may comprise a reverse primer sequence and a 5′ overhang sequence, and the second adapter 3332 may comprise a forward primer sequence and a 3′ overhang sequence, such that subsequent pre-enrichment may be performed in the absence of amplification. Alternatively or in addition to, the ligation of the first adapter 3336 and the second adapter 3332 may maintain the directionality of the initial template sequence. In some instances, the first adapter 3336 and/or the second adapter 3332 comprises a blocked end (e.g., for preventing ligation or extension). See, Example 16.

The overhang sequences of the first adapter 3336 and/or the second adapter 3332 may anneal to a portion of the single-stranded template sequence. Subsequent to annealing, ligation of the adapters to the single-stranded template sequence may be performed to generate a template nucleic acid molecule. In some instances, phosphorylation may be performed, e.g., using an enzyme such as a kinase (e.g., T4 polynucleotide kinase) prior to ligation of the adapters to the single-stranded template sequence. The ligated molecule may subsequently be attached to a support (e.g., bead) 3342, as described elsewhere herein. Alternatively, the template nucleic acid molecule may be attached to the support and then subjected to ligation. Beneficially, utilizing a splint ligation approach may simplify template nucleic acid preparation workflows, e.g., by obviating, in some instances, the need for amplification processes to add the adapter sequences. Such approaches may also allow for ligation of adapters within a single reaction mixture while maintaining directionality of the template sequences. Such an approach may also be compatible with the pre-enrichment operations described herein and, in some instances, obviate the need for amplification prior to sequencing.

In another example, a SPLAT approach may be used to attach adapters to template sequences. In such an example, the template sequences may be subjected to conditions sufficient to generate nicks in the template sequences and/or to fragment the template sequences (e.g., via amplification in the presence of uracil and using a uracil-excising enzyme to excise the uracil, bisulfite conversion of the template sequences, etc.). Other approaches to fragment the template sequences may be performed, e.g., mechanical fragmentation (e.g., shearing, ultrasonication, vortexing, acoustic shearing (e.g., Covaris® shearing), etc.), which may optionally be followed by bisulfite treatment or other enzymatic conversion of non-methylated cytosines to uracil.

Subsequent priming and extension, e.g., in the presence of the first adapter and the second adapter, may be performed to generate a template nucleic acid molecule. Alternatively or in addition to, the adapters may be ligated to an end of the nicked or fragmented template sequence. For example, a first adapter may comprise a sequence (e.g., random N-mer) that may hybridize to a sequence of the template sequence. In some instances, the first adapter may be ligated to a 3′ terminus of the nicked or fragmented template sequence, and prior to, concurrently, or subsequently, a second adapter may be ligated to a 5′ terminus of the nicked or fragmented template sequence, e.g., via hybridization of a complementary sequence (e.g., random N-mer). The template nucleic acid molecule may then be attached to a support, as described herein, and subjected to further processing, e.g., pre-enrichment.

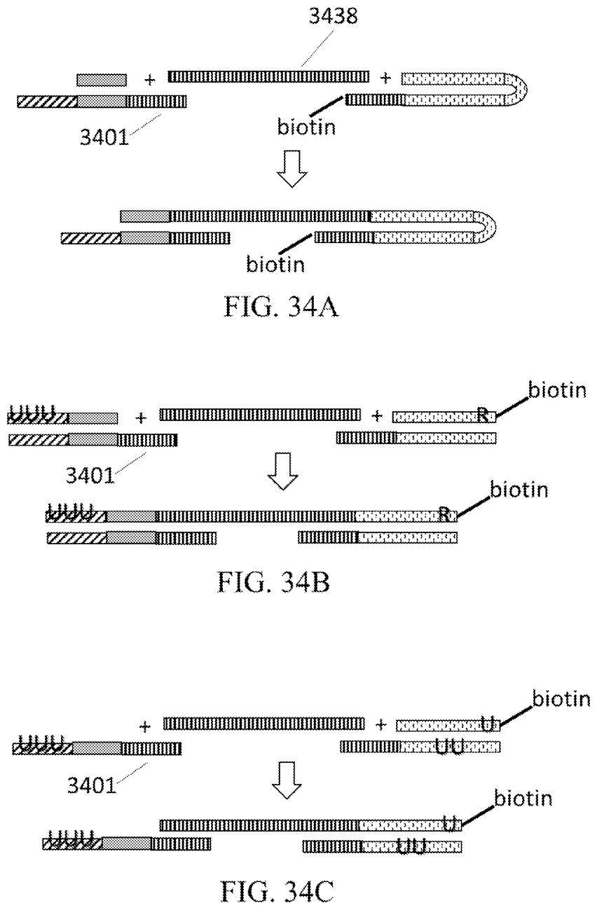

FIG. 34 schematically illustrates non-limiting examples of adapter molecules which may be ligated, e.g., using a splint ligation approach, to a template sequence. FIG. 34A shows a pair of adapters in which a first adapter comprises a hairpin sequence and a capture moiety (e.g., biotin) and a second adapter comprises an overhang sequence 3401 that may anneal to a support. FIG. 34B shows a pair of adapters in which a first adapter comprises a cleavable ribonucleotide sequence and a capture moiety (e.g., biotin) and a second adapter comprises an overhang sequence 3401 that may anneal to a support and a cleavable moiety (e.g., uracil). FIG. 34C shows a pair of adapters in which a first adapter comprises one or more cleavable moieties (e.g., uracil) and a capture moiety (e.g., biotin) and a second adapter comprises an overhang sequence 3401 that may anneal to a support and a cleavable moiety (e.g., uracil). In FIGS. 34A-C, each pair of adapters or each adapter may comprise an overhang sequence capable of annealing to a portion of the template sequence 3438 and optionally, a blocking moiety (e.g., amino group). Subsequent annealing and optional ligation of the adapters to the template sequences 3438 may be performed to generate a template nucleic acid molecule that may subsequently be attached to a support and subjected to pre-enrichment.

It will be appreciated that the first adapter or second adapter can comprise any combination of functional sequences (e.g., primer sequences, sequencing primers) as well as functional moieties (e.g., blocking moieties, cleavable moieties, capture moieties, etc.). For example, the first adapter or the second adapter may be single stranded, double stranded, or partially double stranded. The first adapter or the second adapter may be configured to hybridize to a splint adapter comprising a sequence complementary to a sequence of the first adapter or the second adapter. Any of the adapters may comprise a cleavable moiety (e.g., uracil, ribonucleotide, methylated nucleotide, spacer, etc.), a capture moiety (e.g., biotin, a capture oligonucleotide sequence, etc.), a blocking moiety, or combinations thereof. The first adapter or the second adapter may comprise a blunt end, an overhang sequence, or combinations thereof. Alternatively or in addition to, the first adapter or the second adapter may comprise a complementary sequence (e.g., a binding sequence) that is complementary to a sequence (e.g., capture sequence) on a support, which may allow for attachment of the adapter or (or adapter-ligated template nucleic acid molecule) to the support. See Example 17.

In some instances, hybridization and attachment of the template nucleic acid molecule to the support may be modulated by adjusting the concentration of one or more adapters that are used to generate the template nucleic acid molecule. For example, the ratio of support-to-template nucleic acid molecule attachment may be controlled by controlling the amount of an adapter (e.g., that is ligated to the 5′ end of the template sequence) that is provided. For example, where an excess of template sequences is provided, any useful support-to-template nucleic acid molecule ratio may be achieved by controlling the amount of adapter molecules that is provided. As the adapter molecule is the rate-limiting reagent, using such an adapter-mediated process may obviate the need to quantify or quantitate the number of ligatable template nucleic acid molecules (e.g., UDG-treated template nucleic acid molecules that have 3′ ligatable ends). Since these ligatable molecules may be challenging to quantitate, using the adapter-concentration mediated attachment approach can overcome this challenge, since the attachment ratio of the template nucleic acid molecule to the support is controlled by the adapter concentration. In other instances, the template nucleic acid molecules may be quantitated (e.g., using qPCR) and the attachment ratio to the supports may be controlled using the adapter concentration.

Referring again to FIG. 21, in some instances, subsequent to attachment of template nucleic acid molecules to one or more supports (e.g., 2103) and optionally, pre-enrichment (e.g., 2104), one or more processes may be repeated. For example, during or subsequent to pre-enrichment (2104), a portion of the supports may not comprise a template molecule coupled thereto (e.g., the supports may comprise single-stranded negative supports, also referred to herein as unbound supports). These unbound supports may be recycled or reused, e.g., reintroduced with additional template molecules (2103) and optionally, pre-enriched (2104). Such recycling or reusing of supports is useful in minimizing reagent waste (e.g., such as discarded or unused supports, template molecules, etc.) and may result in higher yield of supports comprising template molecules. Such a recycling process may be repeated any number of times as is useful, e.g., at least 1 time (×), 2×, 3×, 4×, 5×, 10×, 50×, 100×, or more.

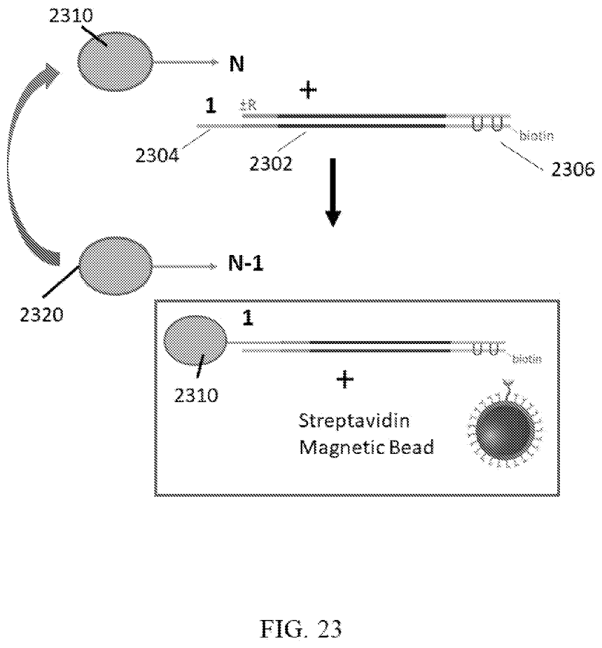

FIG. 23 schematically shows an example process of recycling one or more supports. A first support 2310 and a second support 2320 may be provided with a double stranded template nucleic acid molecule comprising a template sequence. Similar to FIG. 22A, the double stranded template nucleic acid molecule may comprise different sequences at the 5′ and 3′ ends. For example, the 5′ and 3′ ends of a first strand (e.g., strand 2302) of the template nucleic acid molecule may comprise an adapter sequence (e.g., 2304), such as a P1 or P2, or complements thereof, which may be complementary to a sequence of the first support 2310 and/or the second support 2320. The double stranded template nucleic acid molecule may additionally comprise a sequence 2306 with one or more cleavable moieties (e.g., a uracil or ribonucleotide) and an affinity tag (e.g., a biotin moiety). The double stranded template nucleic acid molecule may hybridize and/or be ligated to a sequence (e.g., primer) on the first support 2310. The second support 2320 may remain unbound (e.g., if it does not contact a template nucleic acid molecule). Both the first support 2310 and the second support 2320 may be subjected to pre-enrichment, as described herein, e.g., by providing the supports with a magnetic bead comprising capturing moieties (e.g., streptavidin). The pre-enrichment process may capture the first support 2310, e.g., via the affinity tag. The second support 2320, which does not have a double stranded template nucleic acid molecule or affinity tag, may then be reused or recycled (e.g., pooled with the same or different double stranded template nucleic acid molecules), and the hybridization and/or ligation and pre-enrichment processes may be repeated.

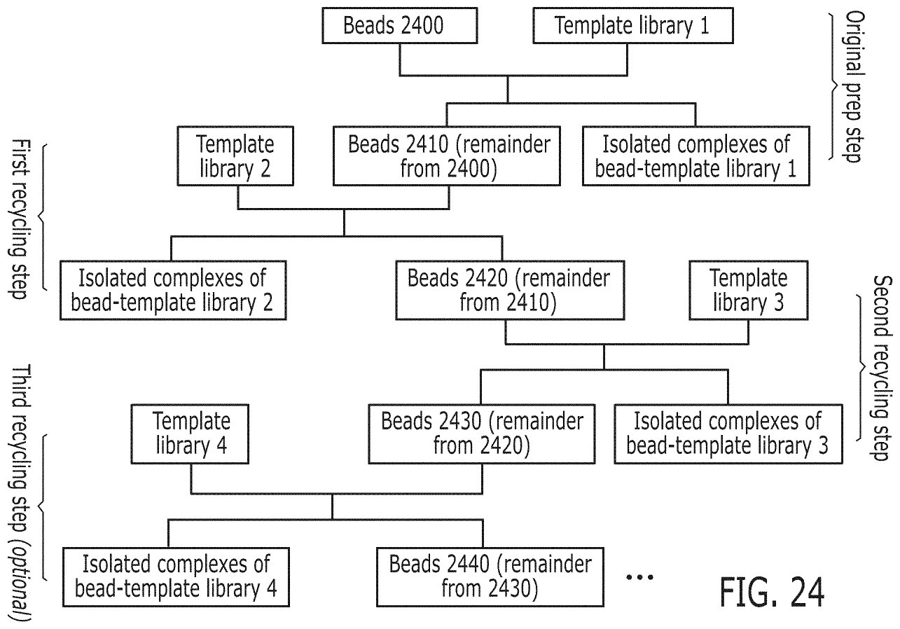

FIG. 24 shows a diagram of an example workflow for recycling unbound supports (e.g., beads) in order to maximize the number of supports bound to a template nucleic acid molecule. A plurality of supports, e.g., beads 2400 are provided with a first plurality of template nucleic acid molecules (e.g., “Template library 1”). Each of the first plurality of template nucleic acid molecules may comprise an affinity tag, e.g., biotin. A portion of the supports 2400 will hybridize and/or be ligated to a template nucleic acid molecule, thereby generating a positive support (e.g., “isolated complex of bead-template template nucleic acid 1”) and can be enriched, e.g., using a capture moiety (e.g., streptavidin). An additional plurality of supports 2410 may be negative or unbound supports and accordingly, will not be captured by the capture moiety during pre-enrichment. This additional plurality of supports 2410 may be available for attachment of a template nucleic acid molecule.

The additional plurality of supports 2410 may be recycled, e.g., by combining them with a second plurality of template nucleic acid molecules (e.g., “template library 2”). The second plurality of template nucleic acid molecules can be the same template nucleic acid molecules as the first plurality of template nucleic acid molecule (e.g., “template library 1”), or the template nucleic acid molecules may be different (e.g., a new set of template nucleic acid molecules). In some instances, the additional plurality of supports 2410 may be provided or contacted with the mixture or solution comprising the first plurality of template nucleic acid molecules. A portion of the additional plurality of supports 2410 will hybridize and/or be ligated to a template nucleic acid molecule of the second plurality of template nucleic acid molecules, thereby generating a positive support (e.g., “isolated complex of bead-template library 2”) and can be enriched, e.g., using a capture moiety (e.g., streptavidin). The unbound supports 2420 are not captured by the capture moiety during pre-enrichment and are thus available for recycling, e.g., hybridizing and/or ligating with a third plurality of template nucleic acid molecules (“template library 3”), or alternatively, any of the remaining template nucleic acid molecules in the first plurality of template nucleic acid molecules (“template library 1”) or the second plurality of template nucleic acid molecules (“template library 2”). It will be appreciated that the support recycling process can be repeated as many times as is useful to produce template nucleic acid-bound supports. For example, FIG. 24 illustrates 5 rounds of template nucleic acid-support attachment and pre-enrichment, but the recycling of the supports may be repeated for fewer or a greater number of rounds. In some cases, the additional plurality of supports may be provided to the template molecules (e.g., either newly provided template molecules or template molecules from an earlier attachment round) as a mixture with a new (e.g., unrecycled) population of supports.

In some instances, after pre-enrichment and prior to recycling of the unbound supports (e.g., re-introducing the unbound supports with template molecules), the unbound supports may be treated with a blocking agent. For example, following collection of the unbound supports, the unbound support fraction may comprise contaminating agents, e.g., free-floating capture moieties (e.g., streptavidin), which may reduce the attachment efficiency of the template nucleic acid molecules to the support or affect pre-enrichment (e.g., reduce capture efficiency of the affinity tag, such as biotin, to the capture moiety, e.g., streptavidin magnetic particle). Accordingly, a blocking agent may be provided to bind to or inhibit the contaminating agents. The blocking agent may comprise a protein or peptide, ions, small molecules, lipids, carbohydrates, or combinations thereof. For example, the blocking agent may comprise biotin or biotin analogs (e.g., biocytin), which may bind to free-floating capture moieties (e.g., streptavidin). Accordingly, the free-floating capture moieties may be prevented from inhibiting downstream pre-enrichment and thus maintain high capture efficiencies of positive supports during pre-enrichment (see, e.g., Example 13).

As described herein, a combination of enzymes may be used to facilitate attachment of the template nucleic acid molecules to a support. For example, double stranded template nucleic acid molecules comprising adaptor sequences may be attached to a support using a ligase and optionally, a polymerase (e.g., Taq polymerase). Use of a polymerase in combination with a ligase may facilitate attachment of the template nucleic acid molecules to supports, e.g., by removing excess cleavable moieties or by rendering the template nucleic acid molecule ligatable. For example, in some instances, the template nucleic acid molecule may lack a ligatable feature (e.g., lacking a 5′ phosphate group), such that following hybridization of the template nucleic acid molecule to a support (or primer coupled to the support), the template nucleic acid molecule cannot be ligated to the support (or the primer coupled thereto). In such instances, a polymerase may be used to generate a phosphorylated end (e.g., 5′ end) of the template nucleic acid molecule, thereby allowing ligation of the template nucleic acid molecule to the support (or primer coupled thereto). In other examples, the polymerase may cleave off excess cleavable moieties (e.g., ribonucleotides or uracil). For example, a template nucleic acid molecule may be generated via amplification using primers comprising the cleavable moieties (e.g., as shown in FIG. 22B). In some instances, the enzymes (e.g., USER or RNase) may not completely remove the cleavable moieties and the template nucleic acid molecule is thus not ligatable to the support (or primer coupled thereto). In such instances, a polymerase may be used to cleave off the excess moieties (e.g., uracil, ribonucleotides) and render the template nucleic acid molecule ligatable to the support (or primer coupled thereto). Use of a polymerase prior to or during the ligation process may increase the number of supports ligated to template nucleic acid molecules (see, e.g., Example 14).

### EXAMPLES

Certain examples of the following examples illustrate various methods of making linkers and labeled substrates described herein. It is understood that one skilled in the art may be able to make these compounds by similar methods or by combining other methods known to one skilled in the art. It is also understood that one skilled in the art would be able to make other compounds in a similar manner as described below by using the appropriate starting materials and modifying synthetic routes as needed. In general, starting materials and reagents can be obtained from commercial vendors or synthesized according to sources known to those skilled in the art or prepared as described herein.

Unless otherwise noted, reagents and solvents used in synthetic methods described herein are obtained from commercial suppliers. Anhydrous solvents and oven-dried glassware may be used for synthetic transformations sensitive to moisture and/or oxygen. Yields may not be optimized. Reaction times may be approximate and may not be optimized. Materials and instrumentation used in synthetic procedures may be substituted with appropriate alternatives. Column chromatography and thin layer chromatography (TLC) may be performed on reverse-phase silica gel unless otherwise noted. Nuclear magnetic resonance (NMR) and mass spectra may be obtained to characterize reaction products and/or monitor reaction progress.

### Example 1

This example demonstrates the production of single bead species according to the scheme illustrated in FIG. 2.

Methods of producing unique bead species includes a bead 200 that comprises a first nucleic acid molecule 201 attached to the surface. Attachment of nucleic acid molecules to the bead surface can include any of the bioconjugation strategies described herein.

In a first step, the bead is contacted with a second nucleic acid molecule 202. A portion 203 of the second nucleic acid molecule is configured to hybridize with a first nucleic acid molecule 201, or a portion thereof. The second nucleic acid molecule further comprises a second portion 204. Following hybridization, the nucleic acid molecules is extended, e.g., as shown in FIG. 2, to generate an extension product 206, e.g., on the surface of the beads. Such extension reaction is used to generate bead-bound primer molecules 207 (in FIG. 2 referred to as “P1”).

In a subsequent step, the bead-bound primer molecules 207 is enriched on a substrate surface, e.g., a planar substrate surface. Such enrichment is performed using a first reactive moiety 205 coupled to one (or both) of ends, e.g., the 3′ end, as shown in FIG. 2. The first reactive moiety is a functional group enabling a bioconjugation reaction with a second reactive moiety. In this case, the first reactive moiety 205 is a biotin moiety. The bead-primer produce comprising the biotin moiety is brought into proximity (e.g., into fluidic contact) with a surface, wherein such surface (e.g., a planar surface) comprises the second reactive moiety, a streptavidin moiety (SA, 208). Only beads comprising the P1 primer are immobilized on the surface.

In a subsequent step, and after removal of beads that do not comprise a unique primer 207, the enriched unique beads can be detached or eluted from the substrate surface to provide a first set of unique beads comprising a single primer species 209 (e.g., an extended primer). This production process can be repeated multiple times to generate a plurality of unique sets of beads.

As shown in FIG. 2, such set of unique beads comprising a single primer species 209 can then hybridize with a single template nucleic acid molecule (e.g., of a library of different template nucleic acid molecules) via an adapter sequence 211 (in FIG. 2 referred to as “P1′”). Upon hybridization, the nucleic acid molecules are extended, amplified, and analyzed, e.g., sequenced.

### Example 2

This example demonstrates the enrichment of amplified bead surface primers to enhance sensitivity of nucleic acid sequencing.

A plurality of beads, e.g., unique beads, each comprising surface primers are brought into fluidic contact with template nucleic acid molecules. The template nucleic acid molecules comprise adapter sequences that allow hybridization with the bead surface primers. Following hybridization and extension, the immobilized template nucleic acid molecules are amplified and the resulting amplicons, of at least subset thereof, are immobilized on the beads. Following amplification, a number of beads comprise template nucleic acid molecules and/or amplicon thereof immobilized to their surface but may also contain remaining “un-reacted” primers (FIG. 18A). Other beads may not contain any template nucleic acid molecules and/or amplicon thereof. Thus, in order to increase the sensitivity during sequencing and reduce artifacts, such “un-reacted” primers are removed prior to sequencing.

In order to remove such “un-reacted” primers, the beads are contacted with a solution containing nucleic acid sequences capable of hybridizing with the unreacted primers to generate double-stranded nucleic acid molecules. Such double-stranded nucleic acid molecules are then enzymatically removed or degraded by, e.g., using an exonuclease such as ExoIII.

With reference to FIG. 18B, such second plurality of single-stranded nucleic acid molecules can be used to generate a plurality of double-stranded nucleic acid molecules that can be coupled to at least a subset of the plurality of supports, wherein the plurality of double-stranded nucleic acid molecules comprises at least a subset of the second plurality of single-stranded nucleic acid molecules coupled (e.g., hybridized) to a second set of unreacted and/or non-amplified single-stranded nucleic acid molecules (e.g., primer molecules) of the first plurality of single-stranded nucleic acid molecules. Thus, the second plurality of nucleic acid molecules can be used to occupy all remaining free and unbound primer molecules of the support surfaces.

In some cases, the first subset of single-stranded nucleic acid molecules of the first plurality of single-stranded nucleic acid molecules (e.g., a first subset of primer molecules) are hybridized to template nucleic acid molecules and thus can be mutually exclusive to the second subset of single-stranded nucleic acid molecules of the first plurality of single-stranded nucleic acid molecules (e.g., a second subset of primer molecules) that are hybridized to at least a subset of single-stranded nucleic acid molecules of the second plurality of single-stranded nucleic acid molecules to form the plurality of double-stranded nucleic acid molecules.

With reference to FIG. 18C, the double-stranded nucleic acid molecules are then selectively removed or degraded from the at least one subset of the plurality of supports to yield a plurality of processed supports that can comprise or consist of positive supports (e.g., comprising one or more template molecules or amplicons thereof) and negative supports, resulting in an enrichment of template molecules and/or amplicons thereof. Removal or degradation of the double-stranded nucleic acid molecules is achieved using an enzyme such as an exonuclease, e.g., an exonuclease III (ExoIII).

### Example 3

This example demonstrates the enrichment (pre-enrichment) of positive beads prior to template sequence analysis using chemoselective surface immobilization of positive beads on substrate surfaces.

In a first experiment, azide-modified nucleotides are incorporated into the complementary strand of a template sequence during primer extension, wherein the template sequence is attached to the bead surface. The resulting positive beads comprising reactive azide moieties are subjected to a substrate (e.g., glass) surface that is modified with cyclooctene moieties. After incubation, the remaining mixture containing negative beads is washed from the substrate surface, resulting in a substrate surface enriched with positive beads (the only beads containing complementary reactive azide moieties.

The positive beads are then detached from the substrate surface and subjected to sequencing.

In a second experiment, azide-modified nucleotides are incorporated into the template sequence by attached an adapter sequence to the 5′ end, wherein the adapter sequence comprises a plurality of azide-modified nucleotides, e.g., about 30-50 azide-modified nucleotides. The resulting positive beads comprising reactive azide moieties are subjected to a substrate (e.g., glass) surface that is modified with cyclooctene moieties. After incubation, the remaining mixture containing negative beads is washed from the substrate surface, resulting in a substrate surface enriched with positive beads (the only beads containing complementary reactive azide moieties.

The positive beads are then detached from the substrate surface and subjected to sequencing.

### Example 4

This example demonstrates a method for creating a clonally amplified bead using an insert library (I) having the same adapter pair (A/A′) attached on each end (see e.g., FIG. 8). As used herein, a prime (′) designates a reverse complement (e.g., A′ is the reverse complement of A). The bead has a few copies of the second primer (X A) attached to it and many copies of the first primer (X). The adapted insert (A′ I A) hybridizes with the second primer and is extended. The extension product is capable of extending further copies of the first primer (X) but not exponentially. Exponential amplification is permitted when the other end of the extended second (or first) primer is also extended using the fourth primer (A B′). Exponential surface amplification can now take place with the many copies of the surface primer (X) and many copies of the solution primer (third primer, B′). Other beads have a different first primer (Z), so extension products created off of the first bead (X) have no added affinity for the second bead (Z). Temperatures, concentrations and other amplification conditions are selected such that the first and second extensions are slow and/or rare events compared to exponential amplification. Temperatures, concentrations and other amplification conditions are selected such that first extension products (X) do not serve as templates for other beads (Z).

### Example 5

Extended supports comprising a template nucleic acid molecule coupled thereto, as described herein, were prepared using the following procedure.

Annealing and extending the library: A reaction mixture containing a final volume of 100 microliters was prepared with the following components/concentrations: 1 10×TAQ polymerase reaction buffer, 8.2 millimolar (mM) of MgCl₂, 12 mM of dNTP, 10 picomolar (pM) of the library (template nucleic acid molecules), 1 micromole/min (U) Taq DNA polymerase, and 6.00×10⁷ beads/microliter. In some embodiments, the reaction mixture further includes glycerol. In some embodiments, the glycerol comprises less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 30%, or less than 35% of the reaction mixture. The reaction mixture was incubated in a thermocycler using the conditions in Table 1:

TABLE 1
Thermocycling conditions

| Step | Temperature | Time | |
|---|---|---|---|
| 1 | 95° C. | 5 | min |
| 2 | 50° C. | 1 | hr |
| 3 | 70° C. | 1 | hr |

| | | |
|---|---|---|
| 4 | 12° C. | soak |

The beads were washed by adding 400 microliters (μL) of TET Buffer (TE pH 8.0, 0.05% Triton X-100). The mixture was vortexed for 30 seconds and spun down at 21,000 revolutions per minute (RPM) for 8 minutes in a centrifuge. The supernatant was removed to leave 100 μL. The beads were washed with 500 μL of 1× SA Bind Buffer (20 mM Tris pH 3.0, 50 mM NaCl, 0.05% Triton X-100). The mixture was vortexed for 30 seconds and spun down at 21,000 RPM for 8 minutes in a centrifuge. The supernatant was removed to leave 100 μL.

Enriching the extended beads: 100 μL of magnetic Streptavidin beads were added to the extended beads. This mixture was mixed and incubated for 1 hour at room temperature. The beads were magnetized on an appropriate magnet until the solution was clear, and the supernatant was removed. The beads were washed with 500 μL of SA Bind Buffer by gentle resuspension. In a second magnetization operation, the beads were magnetized on an appropriate magnet until the solution was clear, and the supernatant was removed. The beads were washed with 500 μL of SA Bind Buffer by gentle resuspension. In a third magnetization operation, the beads were magnetized on an appropriate magnet until the solution was clear, and the supernatant was removed.

Eluting the extended beads: The beads were resuspended in 300 μL of 50° C. Meltoff Buffer (0.1 mol/liter (M) NaOH, 0.05% Triton X-100), and incubated for 5 minutes at 50° C. The mixture was vortexed briefly and the beads were magnetized on an appropriate magnet until the solution was clear. The supernatant containing the beads were removed and retained. In a second melt-off operation, the beads were resuspended in 300 μL of 50° C. Meltoff Buffer (0.1 mol/liter (M) NaOH, 0.05% Triton X-100), and incubated for 5 minutes at 50° C. The mixture was vortexed briefly and the beads were magnetized on an appropriate magnet until the solution was clear. The supernatant containing the beads were removed and retained, and combined with the earlier supernatant containing the beads. The eluted beads were spun down at 21,000 RPM for 8 minutes in a centrifuge, and the supernatant was removed to leave 100 μL. The beads were washed with 500 μL of 1× SA Bind Buffer, and vortexed for 30 seconds. The beads were spun down at 21,000 RPM for 8 minutes in a centrifuge, and the supernatant was removed to leave 100 μL. The beads were washed with 500 μL of TET Buffer, and vortexed for 30 seconds. The beads were spun down at 21,000 RPM for 8 minutes in a centrifuge, and the supernatant was removed to leave 100 μL.

The enriched beads were subsequently used in ePCR procedures.

### Example 6

Table 2 below and FIG. 13 show the results of amplification using pre-enrichment (e.g., enriching a mixture of supports (e.g., beads), prior to clonal amplification, to use isolated and/or concentrated extended support mixtures for amplification) procedures against control procedures in absence of performing pre-enrichment procedures. Amplification was performed on E. coli Library templates and artificial templates.

TABLE 2
Pre-enrichment vs Control Results

| Process | Template | % Enrichment | % Amplification | % Polyclonal |
|---|---|---|---|---|
| Pre-enrichment | E. coli Library | 5 | 95 | N/A |
| Pre-enrichment | Artificial templates | 1.6 | 90 | 13.25 |
| Control | Artificial templates | N/A | 17 | 11 |

FIG. 13 shows in Panel (A) an E. coli library that was subjected to pre-enrichment procedures, in Panel (B) an artificial template library that was subjected to pre-enrichment procedures, and in Panel (C) an artificial template library that was subjected to control procedures (in absence of pre-enrichment). Each graph shows a distribution of count vs. allophycocyanin (APC) fluorescence. In each panel, the horizontal axis depicts fluorescence intensity levels (in log scale) and the vertical axis depicts the number of beads at certain intensity levels. Amplified beads are shown with higher intensity levels. As shown in Panel (A), the E. coli library (pre-enrichment) resulted in 5% enrichment (against a theoretical 10%), and 95.3% of total fluorescence intensity attributed to amplified beads. As shown in Panel (B), the artificial template library (pre-enrichment) resulted in 1.6% enrichment (against a theoretical 10%), and 89.6% of total fluorescence intensity from amplified beads. As shown in Panel (C), the artificial template library (control) resulted in only 16.8% of total fluorescence intensity from amplified beads. Approximately 13.25% of the pre-enrichment artificial template library population resulted in polyclonal amplification. Approximately 11% of the post-enrichment artificial template library population resulted in polyclonal amplification.

### Example 7

Extended supports comprising an extension primer sequence configured to attach to a template nucleic acid molecule (e.g., adapter attached thereto), as described herein, were prepared using the following procedure.

A serial dilution of biotinylated extension primer molecules was prepared from 10 micromolar stock to each of 10 nanomolar (nM), 1 nM, 0.1 nM, and 0.01 nM stocks in 10 millimolar (mM) Tris pH 8.0, and these were further diluted to achieve a final concentration of 1000, 100, 10, and 1 picomolar (pM) in 60 million beads/μL. A biotinylated extension primer molecule comprises a complement of an extension primer sequence. Pre-annealing between primer molecules on the bead and the biotinylated extension primer molecules occurred at 95° C. for 2 minutes. The mixture was slowly cooled to 50° C. and held for a total of 45 minutes in 1× EpiMark® buffer. The primer was extended for 20 minutes at a 70° C. heat block and washed twice with 1× BW buffer. Magnetic Streptavidin beads were hybridized with the biotin-templated beads for 1.5 hours on rotor at room temperature. The beads were magnetically captured. After magnetic capture, the beads (with single extension primer sequence) were eluted using 0.1% NaOH and 0.05% and Triton X-100 in water at 50° C. for 5 minutes. The enriched beads were washed three times using 1× EpiMark® buffer and subsequently used in ePCR procedures.

### Example 8

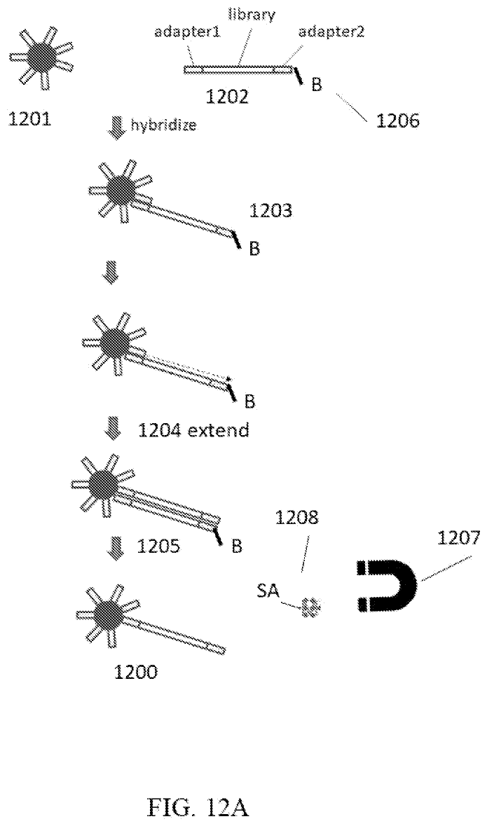

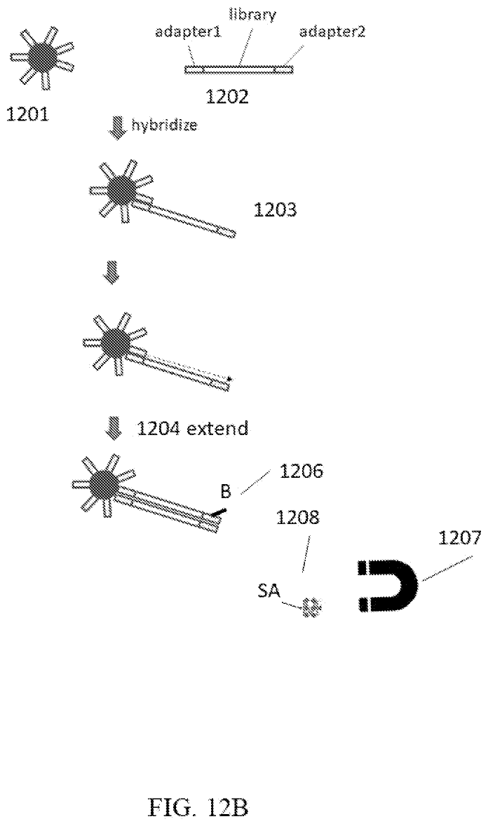

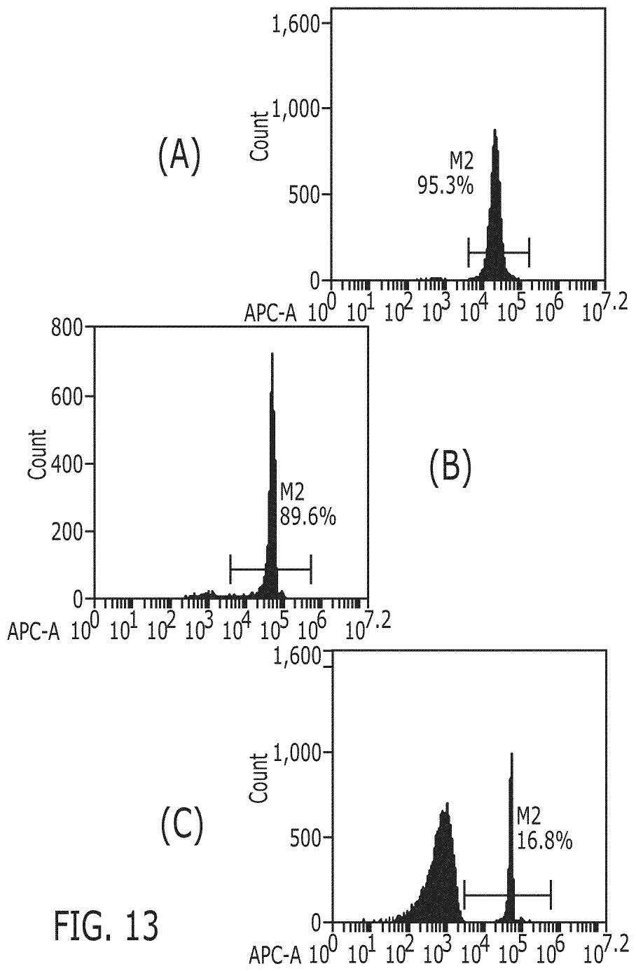

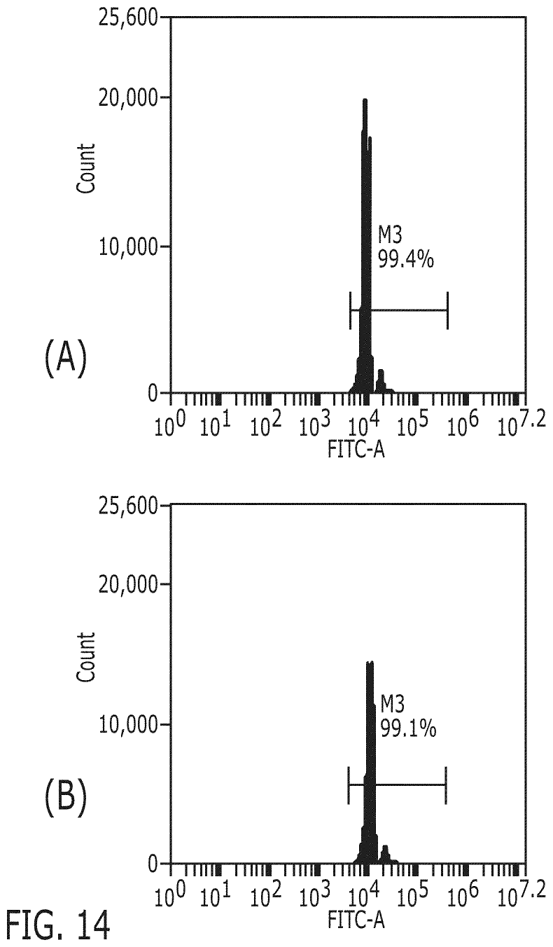

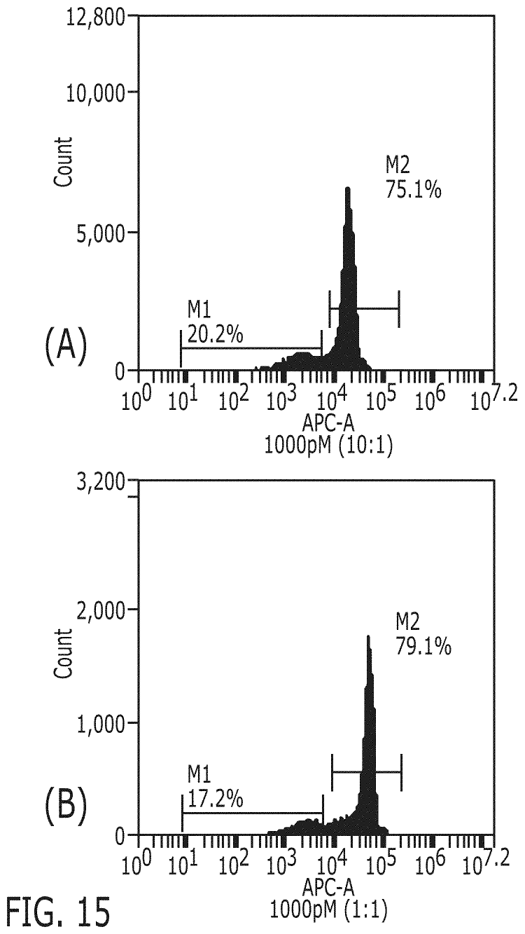

Table 3 below and FIGS. 14-15 show the results of enriched beads captured after primer extension at different extension primer:bead input ratios.

TABLE 3
Capture of Enriched Beads

| Extension | Extension | Predicted % of beads | | | | |
|---|---|---|---|---|---|---|
| Primer | Primer:Bead | with N ext. primers | | | Predicted % of | Observed % of |
| (pM) | (ratio) | N = 0 | N = 1 | N = 2+ | Beads captured | Beads captured |
| 1000 | 10:1 | 0% | 0% | 100% | 100% | 51% |
| 100 | 1:1 | 37% | 37% | 26% | 63% | 35% |

For 1000 pM concentration of extension primers, and extension primer:bead ratio of 10:1, the predicted % for beads with 0, 1, and 2+ templates are 0%, 0%, and 100%, respectively. Accordingly, the predicted % of beads captured (having at least N=1 extension primer) is 100%. The observed % of beads captured was 51%.

For 100 pM concentration of extension primers, and extension primer:bead ratio of 1:1, the predicted % for beads with 0, 1, and 2+ templates are 37%, 37%, and 26% respectively. Accordingly, the predicted % of beads captured (having at least N=1 extension primer) is 63%. The observed % of beads captured was 35%.

FIG. 14 shows in Panel (A) the presence of enriched beads captured at 1000 pM extension primer input concentration, and in Panel (B) the presence of enriched beads captured at 100 pM extension primer input concentration. Each graph shows a distribution of count vs. fluorescein isothiocyanate (FITC) fluorescence, with 800 FITC threshold.

FIG. 15 shows in Panel (A) the presence of the extension primer sequence in enriched beads, at 1000 pM extension primer input concentration and 10:1 extension primer:bead ratio, and in Panel (B) the presence of the extension primer sequence in enriched beads, at 100 pM extension primer input concentration and 1:1 extension primer:bead ratio. Each graph shows a distribution of count vs. allophycocyanin (APC) fluorescence. As shown in Panels (A) and (B), 75.1% and 79.1% of the enriched beads, respectively, were observed to contain at least one extension primer sequence.

### Example 9

Extended supports comprising an extended primer sequence were attached to a template nucleic acid molecule (e.g., adapter attached thereto), as described herein, using the following procedure.

Pre-annealing library template to an extended support: A mixture of two species of single stranded templates and extended beads (a bead comprising the extended primer sequence) was provided at 20-fold excess of template:enriched bead and left to anneal at 95° C. for 2 minutes, slowly cooled to 50° C., and held for a total of 45 minutes in 1× EpiMark® buffer. The mixture was incubated additional times at 50° C. while rotating for 2-20 hours. The beads were washed once with 1× EpiMark® buffer. The resulting beads have a template molecule (e.g., single template molecule) coupled thereto.

Partitioning templated extended support for ePCR: The templated beads were partitioned into droplets for ePCR. It will be appreciated that prior to partitioning for ePCR, the templated bead (e.g., bead coupled to a template molecule via an extension primer sequence) may be coupled to the template via annealing of the template to the extension primer sequence and/or via extension from the extension primer sequence to generate a complement of the template coupled to the bead.

### Example 10

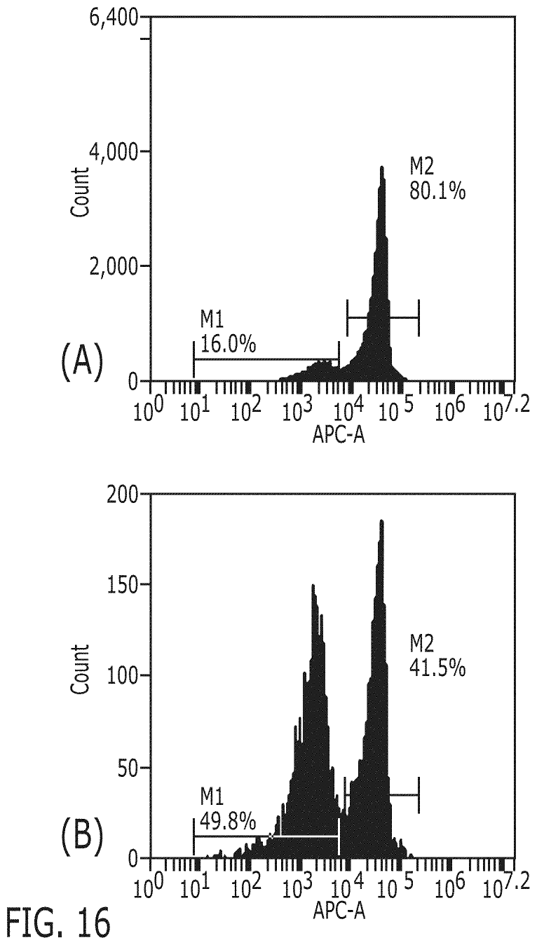

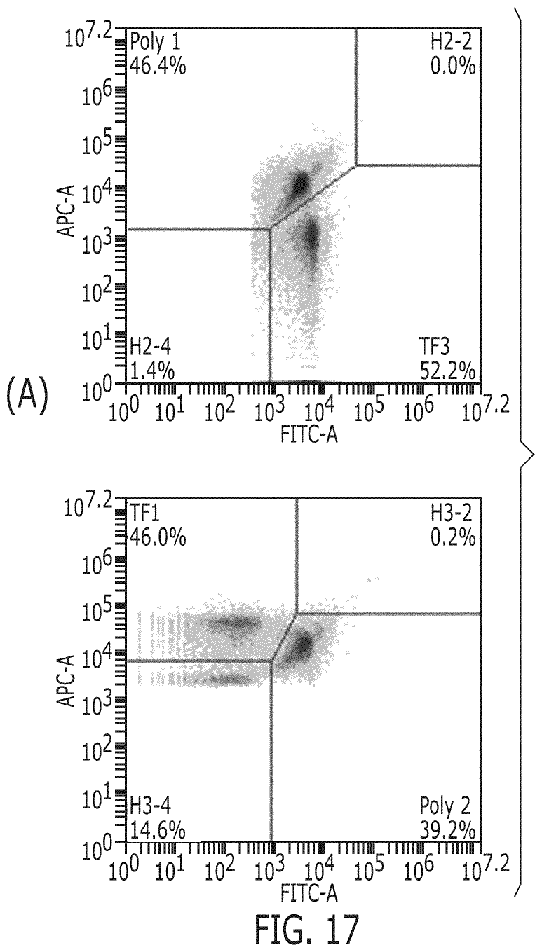

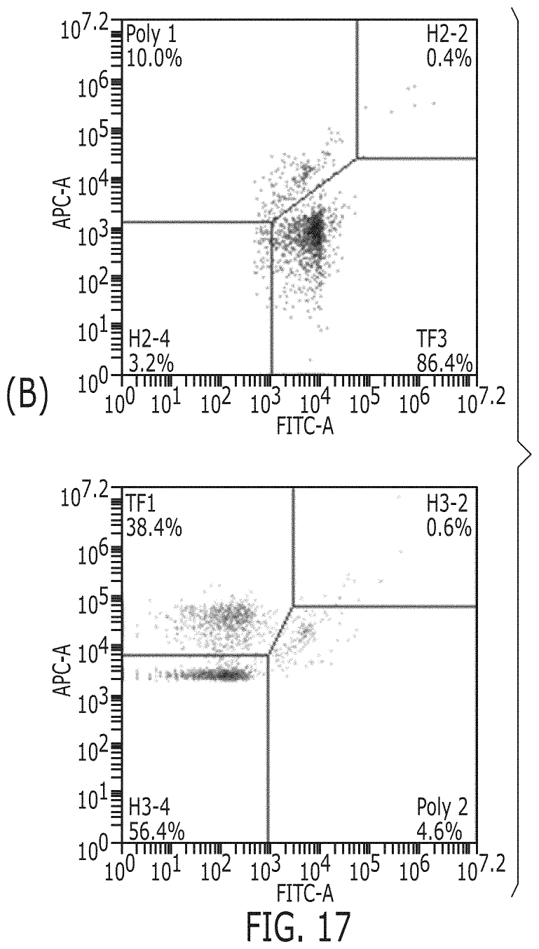

Table 4 below and FIGS. 16-17 show the results of ePCR amplification using templated beads at different extension primer:bead input ratios. Atto probes for two species of templates were annealed to the amplified beads and total amplification measured.

FIG. 16 shows in Panel (A) the presence of amplified beads (or positive beads) at 1000 pM extension primer input concentration, 10:1 extension primer:bead input ratio, 200 pM template input concentration, and 1:20 enriched bead:template input ratio, and in Panel (B) the presence of amplified beads (or positive beads) at 100 pM extension primer input concentration, 1:1 extension primer:bead input ratio, 200 pM template input concentration, and 1:20 enriched bead:template input ratio. Each graph shows a distribution of count vs. allophycocyanin (APC) fluorescence. As shown in Panels (A) and (B), enriched and amplified beads count for 80.1% and 41.5% of the total fluorescence intensity level, respectively, for 1000 pM and 100 pM extension primer input concentrations.

FIG. 17 shows in vertical Panel (A) two graphs indicative of polyclonality of amplified beads at 1000 pM extension primer input concentration, 10:1 extension primer:bead input ratio, 200 pM template input concentration, and 1:20 enriched bead:template input ratio, and in vertical Panel (B) two graphs indicative of polyclonality of amplified beads at 100 pM extension primer input concentration, 1:1 extension primer:bead input ratio, 200 pM template input concentration, and 1:20 enriched bead:template input ratio. Each graph shows a distribution of APC fluorescence vs FITC fluorescence. The two top graphs have a threshold on the FITC, and the two bottom graphs have a threshold on the APC fluorescence.

TABLE 4
Polyclonal %

| Extension Primer | | | |
|---|---|---|---|
| Concentration (pM) | Template Concentration (pM) | Predicted % | Observed % |
| (Bead:Extension Primer) | (Enriched Bead:Template) | Polyclonal | Polyclonal |
| 1000 | 200 | 67% | 30% |
| (1:10) | (1:20) | | |
| 100 | 200 | 23% | 6% |
| (1:1) | (1:20) | | |

As shown in Table 4, the observed polyclonal percentage for pre-enriched beads was much lower at 30% and 6% (for 1000 pM and 100 pM extension primer input concentrations, respectively) than theory predicts at 67% and 23% polyclonality, respectively. Furthermore, the predicted polyclonal percentage for performing ePCR without pre-enrichment is 44% and 22% for 80.1% and 41.5% library-positive rates (see, e.g., positive bead results with respect to FIG. 16), respectively. Accordingly, results showed that performing the pre-enrichment procedures described herein generates lower levels of polyclonality at a given rate of positive beads than with standard Poisson loading (without pre-enrichment).

### Example 11

Table 5 below summarizes data of pre-enrichment experiments based on the scheme depicted in FIG. 22A.

TABLE 5
Pre-enrichment by ligating template nucleic
acid molecules to surface primers on beads

| | | | % of beads | % of beads |
|---|---|---|---|---|
| Cleavable | Total beads | Approximate | expected to be | actually |
| base type | (Millions) | [bead]:[template] | pre-enriched | pre-enriched |
| Uracil | 2079.8835 | 10:1 | 10% | 12.6 |
| | 1741.4775 | | 10% | 10.6 |
| | 1779.96 | | 10% | 10.8 |
| | 1834.121 | | 10% | 11.1 |
| | 2032.938 | | 10% | 12.3 |
| | 1554.9435 | | 10% | 9.4 |
| Ribonucleotide | 376.936 | 10:1 | 10% | 4.79 |
| | 360.877 | | 10% | 4.59 |
| | 361.513 | | 10% | 5.95 |
| | 321.2065 | | 10% | 5.28 |

The enrichment conditions for these different pre-enrichment experiments are similar. Commercial ISP beads at 60M/ul were mixed with template nucleic acid molecules at about 6M/ul.

In some embodiments, a reaction mixture for these experiments has a final volume of 250 microliters into which 5 picomolar (pM) of the template nucleic acid (from artificial templates) was mixed with 6.00×10⁷ beads/μL in the presence of Taq DNA polymerase. The mixture is then incubated at 45 degrees Celsius for 1 hour.

To enrich the beads bound with templates, the 250 μL of ligation reaction was diluted in 750 ul reaction buffer and mixed with 250 μL of streptavidin magnetic beads. This mixture was mixed and incubated for 2 hours at room temperature. The template bound beads (e.g., either a single bead type or multiple types of beads) would have the magnetic beads attached and were selected by an appropriate magnet (or magnets) until the solution was clear (e.g., all the beads are removed from solution), and the supernatant was then removed. The beads are washed in buffer by gentle resuspension. Each wash is followed by a magnetization operation, in which the beads are magnetized on an appropriate magnet (or magnets) until the solution is clear, and the supernatant is removed.

The enriched beads can be eluted by enzymatic cleavage. The mix of amplification product containing uracils and magnetic beads was resuspended in 100 μL of buffer and incubated at 37 degrees Celsius for 30 minutes. In experiments where ribonucleotides or RNA bases were used in constructing the template nucleic acid molecules, RNase HIT was added to the template bound beads and incubated at 37 degrees Celsius for 30 minutes. In experiments where uracil bases were used in constructing the template nucleic acid molecules, USER enzyme was added to the template bound beads and incubated at 37 degrees Celsius for 30 minutes. After enzyme cleavage of the RNA bases or uracils, template bounds beads were released from magnetic beads, leaving avidin bound to streptavidin on the magnetic beads.

Flow cytometry was used to quantify the input beads and the beads recovered after pre-enrichment. Pre-enrichment using strand dissociation without enzymatic cleavage results in 1.6% enrichment (against a theoretical 10%—e.g., a theoretical maximum of 10% of the beads can be bound to template strands since a 10× excess of beads are added to the template solution). In contrast, a cleavage protocol using USER cleavage of uracils (e.g., corresponding to the schematic depicted in FIG. 22A) resulted in about 10% of the input beads being recovered after pre-enrichment, close to the theoretical maximum and indicating high efficiency. RNase H treatment produced approximately a 5% of input beads being enriched. This suggests that an alternative approach corresponding to the schematic depicted in FIG. 22A (except RNA bases instead of uracils were included in the adapters) also worked, albeit at a lower efficiency. These cleavable moiety methods, thus, can produce results significantly superior to the dissociation method.

The enriched beads according to any of the protocols described above can be subsequently used in emulsion PCR procedures. Libraries that were pre-enriched on beads following the procedures described above were found to be functional for downstream purposes.

### Example 12

Cleavable moieties may be useful during or following pre-enrichment (e.g., 2104) and enable, for example, removal or elution of a support-bound template nucleic acid molecule following capture and/or enrichment. One example of a cleavable moiety is a C3 linker, which may be suitable as a substrate for an endonuclease such as APE1.

To evaluate the activity of the APE1 enzyme on various substrates, the following two synthetic oligonucleotide substrates were used:

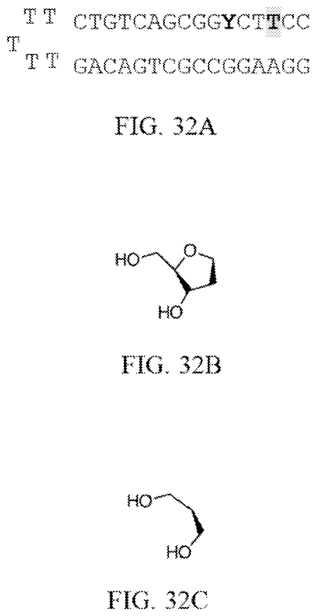

(SEQ ID NO: 1)
Oligo #1: 5′ ACGGTTTCTCCATGXCACCTCC

Where X represents a C3 linker (also referred to as C3 spacer), and the third T residue from the 3′ end is modified by attachment of fluorescein. This oligo was used together with its fully complementary partner, the unmodified Oligo #3 as described below.
Oligo #2, a hairpin-type oligo is shown in FIG. 32A, where Y is a dSpacer residue (FIG. 32B) and the first T residue from the top-right hand side is modified by attachment of fluorescein. Both oligos were obtained from IDT. The chemical structures of the two modified residues are shown below. The structure of the C3 linker is shown in FIG. 32C.

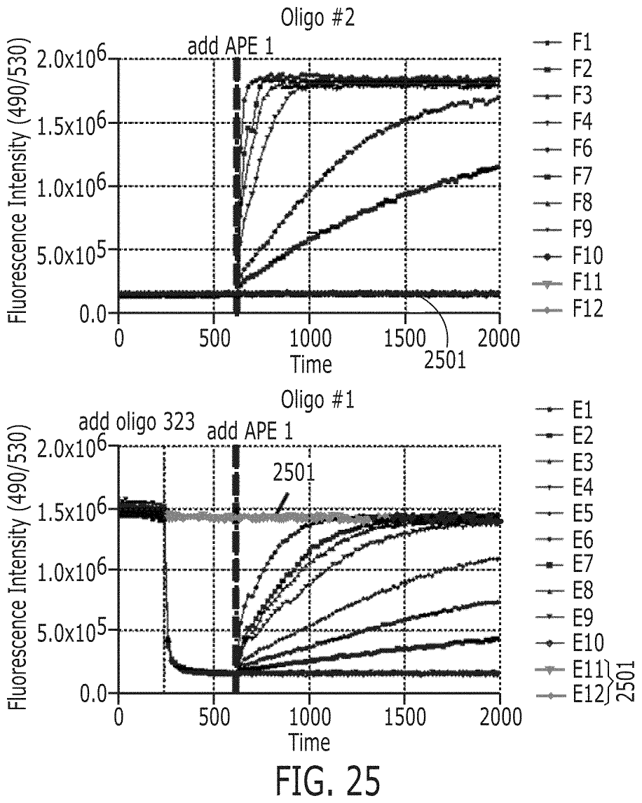

To perform the assay, 20 nM solutions of the two modified substrate oligos (Oligos #1 and #2) were prepared in 1× buffer 4 (NEB). Aliquots (50 μL) of these solutions were placed in individual wells of a 96 well plate and the plate was placed in a fluorescence plate reader, equilibrated at 37° C. The fluorescence emission of the fluorescein attached to the oligos was recorded continuously. To the wells containing the single stranded Oligo #1 was added an excess of its complementary partner, Oligo #3 (the time of addition is indicated by a black dotted line in FIG. 25). Upon formation of the double stranded hybrid, the fluorescein of Oligo #1 is quenched and its intensity decreases. When a constant fluorescence level was reached, 2 μl of serial two-fold dilutions of the enzyme APE1 in buffer 4 were added to the first eight wells of each row of the plate (thick dotted line in the Figure). The highest final concentration of the enzyme (in wells E1 and F1, respectively) was 0.04 units/μl. No enzyme was added to wells E11, E12, F11, and F12 (2501). The changes in fluorescence were recorded. An increase in fluorescence indicates successful cleavage of the substrates by APE1 at the modified positions. Such cleavage releases short, fluorescein labeled fragments (dSpacer-CTTCC and C3 linker-CACCTCC, respectively) which do not undergo quenching and are thus more highly fluorescent. As can be seen in FIG. 25, both oligos are functional substrates for the enzyme APE1, with the dSpacer-modified oligo #2 exhibiting somewhat faster cleavage kinetics than the C3 linker-containing hybrid of Oligos #1 and #3 (approx. 4-fold difference). In control experiments with oligonucleotides not containing the two linker moieties, no change in fluorescence was detected under identical conditions (not shown), thus demonstrating the specificity of the enzymatic reactions. In other experiments, it was also demonstrated that the APE1 enzyme is capable of cleaving at the C3 linker position within single-stranded oligos as well (not shown).

### Example 13

Recycling of supports may be performed to maximize the number of supports attached to template nucleic acid molecules (e.g., as described with respect to FIG. 23).

A summary of experimental results from using biocytin to capture free-floating streptavidin during bead recycling is shown in Table 6.

TABLE 6
Pre-enrichment (PE) efficiencies with recycled beads using
biocytin to capture free-floating streptavidin (SA)

| PEBID | Sample Info | Total Beads | % PE Efficiency |
|---|---|---|---|
| 1st Ligation | Master Mix | 125,385,000,000 | |
| PEB332 | Wash 1 | 276,093,750 | |
| | Wash 2 | 43,125,000 | |
| | SA w/SA | 2,282,000,000 | |
| | SA Elute | 1,216,138,750 | |
| | Elution | 23,158,750,000 | 18.50% |
| Re-ligation 200 pM | Master Mix | 38,114,760,000 | |
| Biocytin | | | |
| PEB333 | Unbound | 29,215,350,000 | |
| | Wash 1 | 105,000,000 | |
| | Wash 2 | 25,000,000 | |
| | SA w/SA | 364,750,000 | |
| | SA Elute | 227,357,500 | |
| | Elution | 6,334,837,500 | 16.60% |
| Re-ligation 100 pM | Master Mix | 41,309,040,000 | |
| Biocytin | | | |
| PEB334 | Unbound | 41,824,125,000 | |
| | Wash 1 | 130,625,000 | |
| | Wash 2 | 28,125,000 | |
| | SA w/SA | 266,750,000 | |
| | SA Elute | 158,951,250 | |
| | Elution | 4,306,950,000 | 10.40% |

As shown in Table 6, an initial population of beads (“PEB332”) is ligated to template nucleic acid molecules at a 1:5 template nucleic acid molecule-to-bead ratio. The master mix contains 125,385,000,000 beads. Following ligation, wash steps, enrichment using streptavidin (SA) magnetic particles, 23,158,750,000 enriched beads comprising template nucleic acid molecules is obtained, which corresponds to a 18.5% capture (pre-enrichment) efficiency. The remaining unbound beads are split into two populations of beads (“PEB333” and “PEB334”). Each of the two populations of beads are incubated with either 200 pM biocytin or 100 pM biocytin, which may bind to free-floating streptavidin, and then re-added to a portion of the master mix and counted. One population (“PEB333”) contains 38,114,760,000 beads and the other (“PEB334”) contains 41,309,040,000 beads. Following ligation, wash steps, enrichment using streptavidin (SA) magnetic particles, 6,334,837,500 beads and 4,306,950,000 beads, respectively, are counted using flow cytometry, which corresponds to a 16.6% and 10.4%, respectively, capture or pre-enrichment efficiency. These results indicate that biocytin may be titrated, e.g., based on the concentration of free-floating streptavidin, to improve pre-enrichment efficiency.

TABLE 7
Pre-enrichment (PE) efficiencies with recycled beads using
150 pM biocytin to capture free-floating streptavidin (SA)

| | Sample Info | Bead Yield (B) | % PE Efficiency |
|---|---|---|---|
| Control | Ligation Mix | 175.52 | |
| | Elution | 23.87 | 13.6% |
| Re-ligation 1 | Ligation Mix | 159.13 | |
| | Elution | 17.13 | 10.8% |
| Re-ligation 2 | Ligation Mix | 147.79 | |
| | Elution | 10.44 | 7.1% |

TABLE 8
Pre-enrichment (PE) efficiencies with recycled beads using
5 nM biocytin to capture free-floating streptavidin (SA)

| | Sample Info | Bead Yield (B) | % PE Efficiency |
|---|---|---|---|
| Control | Ligation Mix | 177 | — |
| | Elution | 23.4 | 13.2% |
| Re-ligation-1 | Ligation Mix | 168.48 | — |
| | Elution | 17.81 | 10.6% |
| Re-ligation-2b | Ligation Mix | 140.22 | — |
| | Elution | 17.44 | 12.4% |

Tables 7-8 also show example data of using biocytin to capture free-floating streptavidin during bead recycling. In each of Tables 7-8, a population of beads (“control”) is mixed with template nucleic acid molecules at a ratio of 1:5 template nucleic acid molecule-to-bead ratio. The initial number of beads are counted (e.g., (“Bead Yield”) in Table 7, displayed as billions of beads). Following ligation, wash steps, and enrichment using streptavidin (SA) magnetic particles, the enriched beads are quantified and normalized to the initial starting number of beads to yield the pre-enrichment efficiency (% PE efficiency). The remaining unbound beads are incubated with biocytin at either a 150 pM concentration (Table 7), or a 5 nM concentration (Table 8), which may bind to free-floating streptavidin, and then re-added to a portion of the master mix and counted. Following ligation, wash steps, enrichment using streptavidin (SA) magnetic particles, the pre-enriched (captured) beads are counted using flow cytometry, and the PE efficiency is counted. The remaining unbound beads are recycled again, and the process is repeated.

As shown in Table 7, the pre-enrichment efficiency drops with each round of re-ligation. However, when the biocytin concentration is increased to 5 nM, as shown in Table 8, the overall pre-enrichment efficiency is improved.

In combination with some modifications of the ligation steps and streptavidin enrichment steps, the pre-enrichment bead usage efficiency can be maintained over more rounds of ligation (e.g., at least 3, 4, 5, or more rounds of ligation). Specifically, ligation incubation times can be modified to be less than about 60 min, 30 min, 20 min, 15 min, 10 min, 5 min, or 2 min. Likewise, streptavidin enrichment incubation times can be modified to be less than about 60 min, 30 min, 20 min, 15 min, 10 min, 5 min or 2 min.

TABLE 9
Pre-enrichment (PE) efficiencies with recycled beads
using 5 nM biocytin to capture free-floating streptavidin
(SA) and modified incubation times

| | | Reaction A | | Reaction B | | Reaction C | |
|---|---|---|---|---|---|---|---|
| | | Bead | PE | Bead | PE | Bead | PE |
| | | Yield | effi- | Yield | effi- | Yield | effi- |
| Sample Info | | (B) | ciency | (B) | ciency | (B) | ciency |
| Ligation 1 | Ligation | 149.0 | | 153.0 | | 180.6 | |
| | Mix | | | | | | |
| | Elution | 13.7 | 9.2% | 14.0 | 9.2% | 19.7 | 10.9% |
| Ligation 2 | Ligation | 141.2 | | 148.8 | | 178.4 | |
| | Mix | | | | | | |
| | Elution | 13.9 | 9.8% | 13.8 | 9.3% | 19.2 | 10.8% |
| Ligation 3 | Ligation | 122.2 | | 142.5 | | 144.8 | |
| | Mix | | | | | | |
| | Elution | 11.7 | 9.6% | 13.4 | 9.4% | 16.2 | 11.2% |
| Ligation 4 | Ligation | 115.3 | | 129.2 | | 134.0 | |
| | Mix | | | | | | |
| | Elution | 10.0 | 8.7% | 9.9 | 7.6% | 14.7 | 11.0% |

As illustrated in Table 9, pre-enrichment efficiency is maintained at similar levels over at least four rounds of ligation re-using beads. For example, in Reaction A the pre-enrichment efficiency ranges from 9.8% to 8.7%; for Reaction B pre-enrichment efficiency ranges from 9.4% to 7.6%; and for Reaction C pre-enrichment efficiency ranges from 11.2% to 10.8%. Altogether, the results indicate that incubating beads, following pre-enrichment, with a titrated concentration of biocytin to capture free-floating streptavidin enables recycling (e.g., utilizing overall a higher percentage of the initial population) of beads (e.g., supports) without reducing pre-enrichment efficiency.

### Example 14

In some instances, when attaching template nucleic acid molecules to supports (e.g., beads), a polymerase (e.g., Taq polymerase) and nucleotides (e.g., dNTPs) may be provided in addition to a ligase. Such a process may precede pre-enrichment; subsequent pre-enrichment efficiency may be calculated (e.g., amplifying sequences obtained from captured or enriched supports, counting the number of supports comprising amplified sequences, and normalizing to the number of starting supports). The pre-enrichment efficiency may be used to determine the ligation efficiency of the template nucleic acid molecules to the supports.

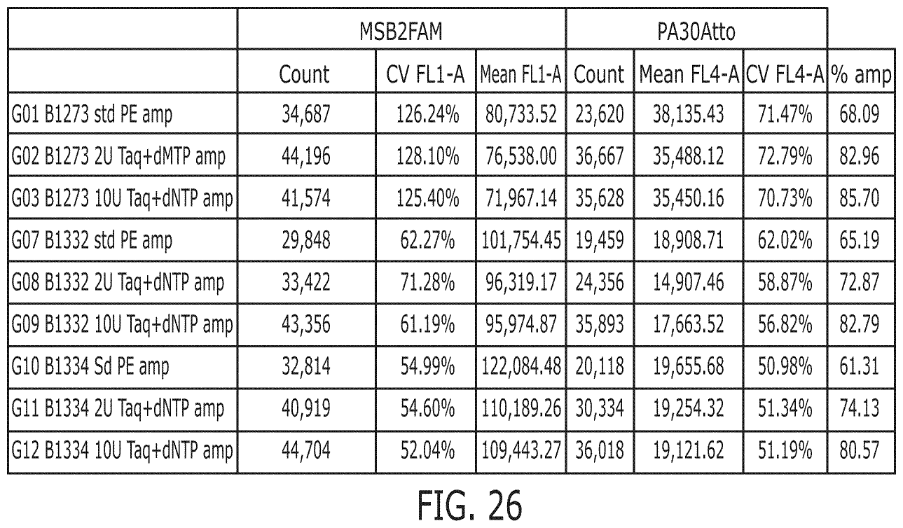

FIG. 26 illustrates example data of the pre-enrichment efficiencies (% amp) of bead samples (G01 to G12) with templates ligated thereto without Taq (“std PE amp”) and with the addition of Taq polymerase at a concentration of 2 units or 10 units, along with nucleotides. B1273, B1332, and B1334 indicate different bead types. As illustrated in FIG. 26, the pre-enrichment efficiency (% amp) increases for multiple bead types (B1273, B1332, and B1334). For example, for B1273, the pre-enrichment efficiency, which may be indicative of the number of ligated templates to the beads, increases from 6.09% to 82.96% (for 2 units Taq) and 85.70% (for 10 units Taq). Similar increases in efficiency are perceived in the B1332 and B1334 beads.

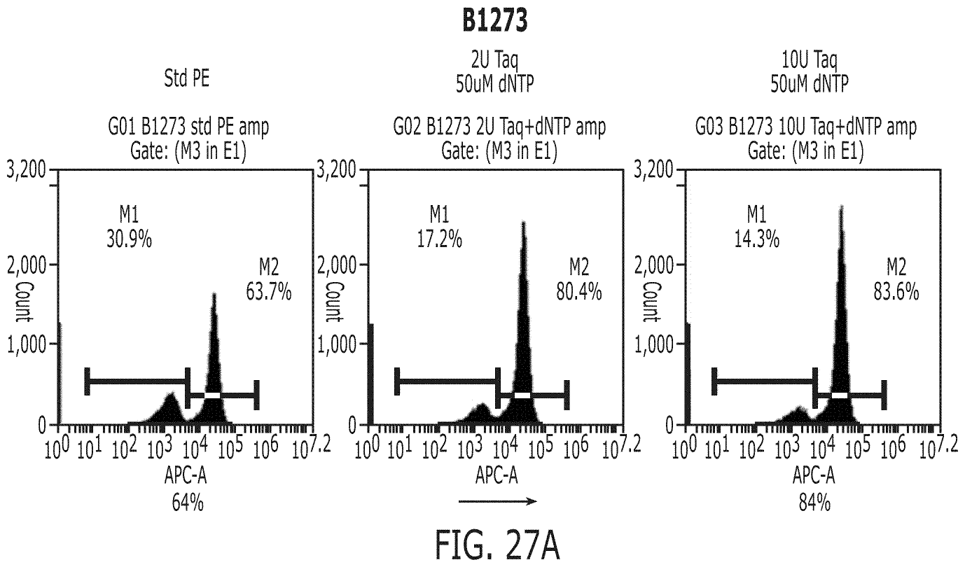

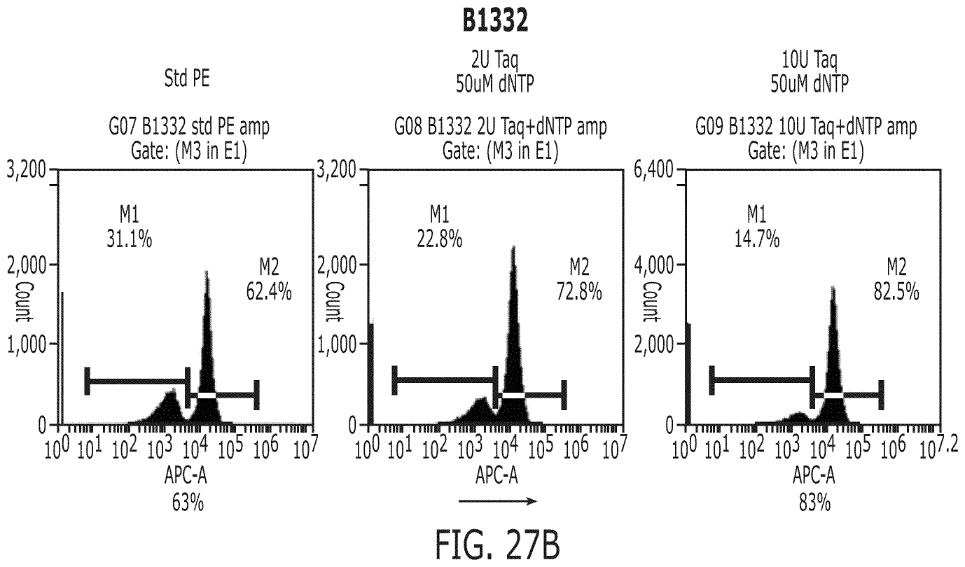

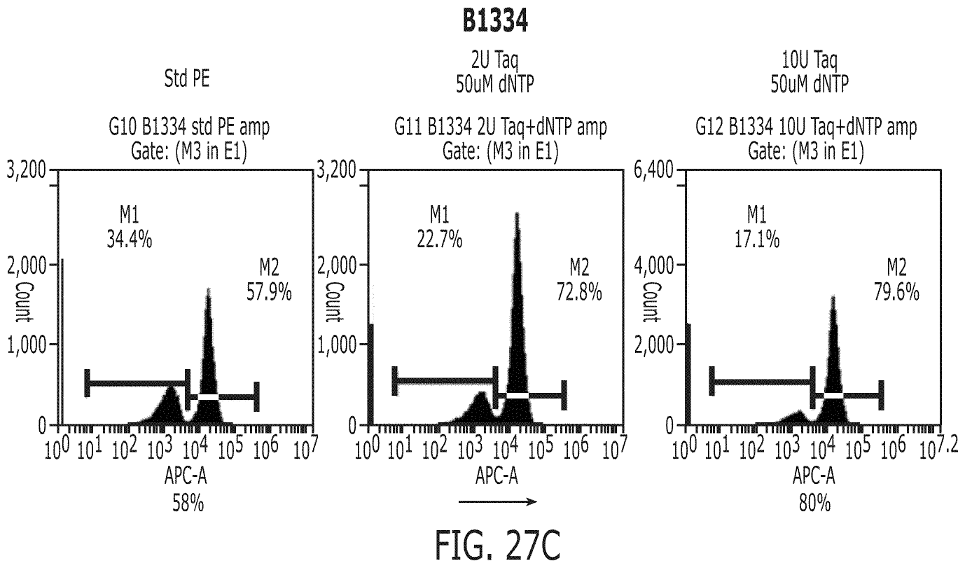

FIGS. 27A-27C show flow cytometry histograms (counts of fluorescence intensity of individual supports) of each bead type shown in FIG. 26. For each plot, the first peak (left-hand peak) indicates a non-amplified support (e.g., support lacking a template nucleic acid molecule ligated thereto), and the second peak (right-hand peak) indicates an amplified support. FIG. 27A shows flow cytometry results for bead B1273, FIG. 27B shows flow cytometry results for B1332, and FIG. 27C shows flow cytometry results for B1334.

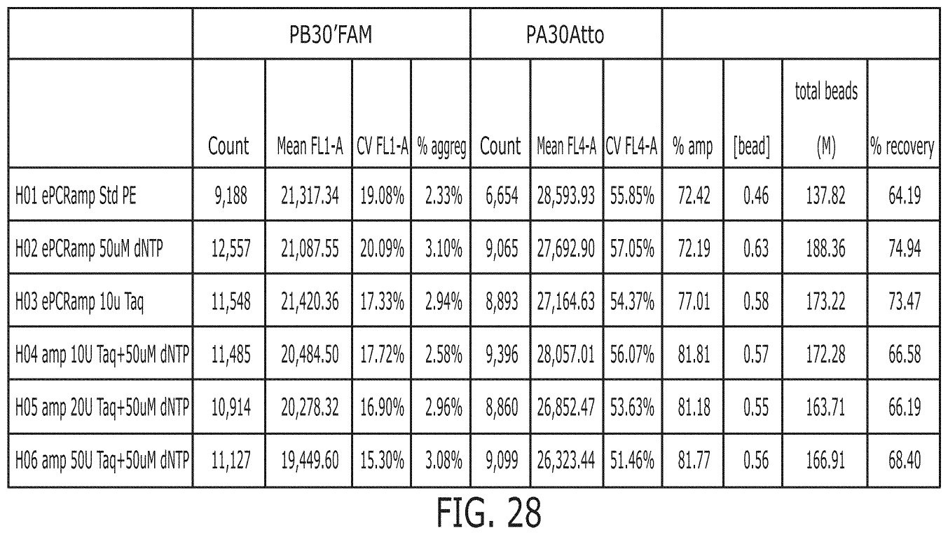

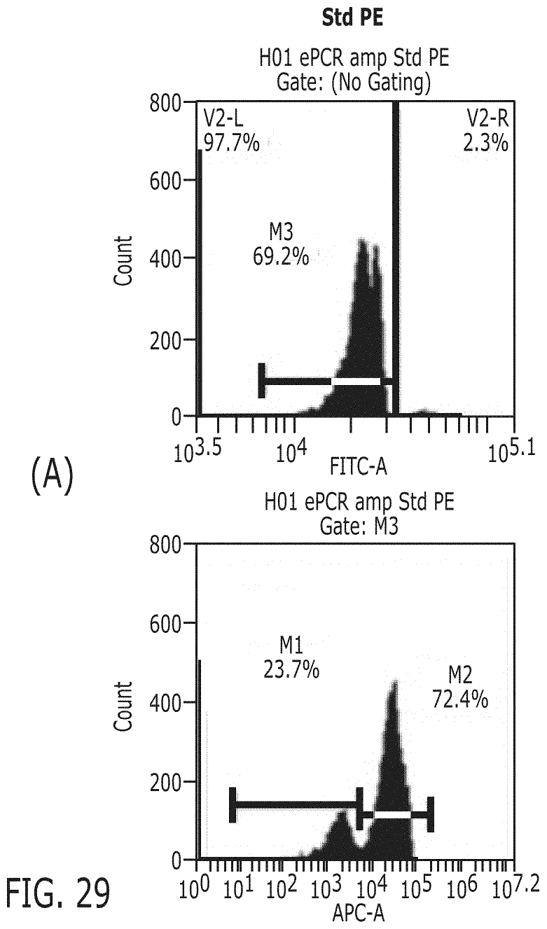

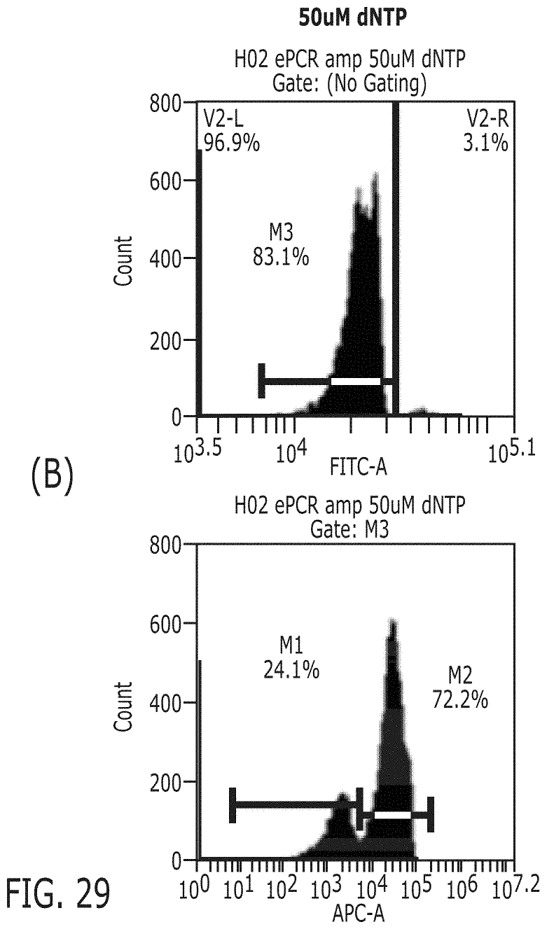

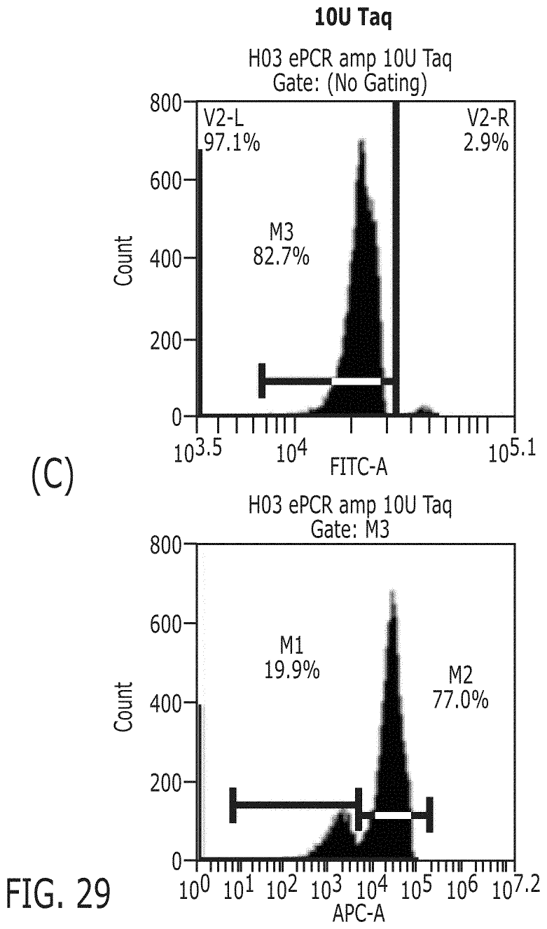

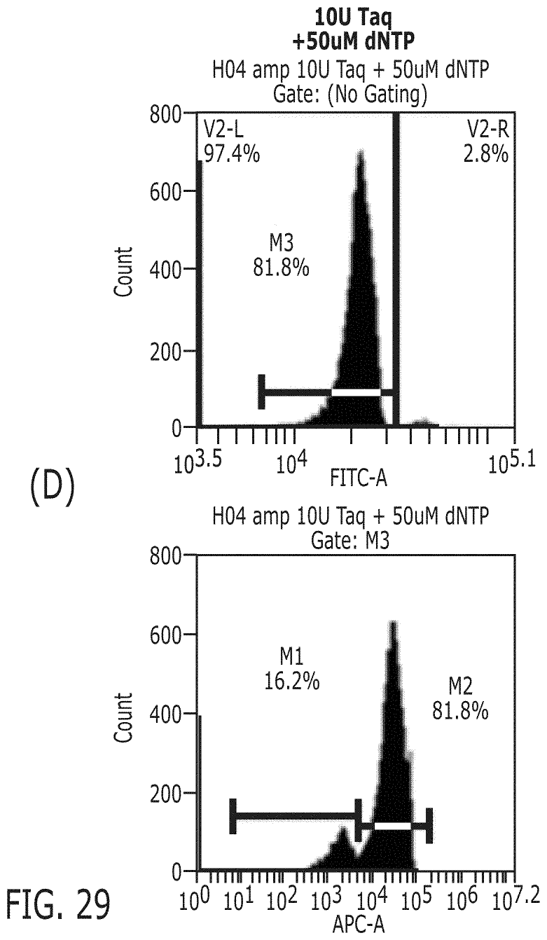

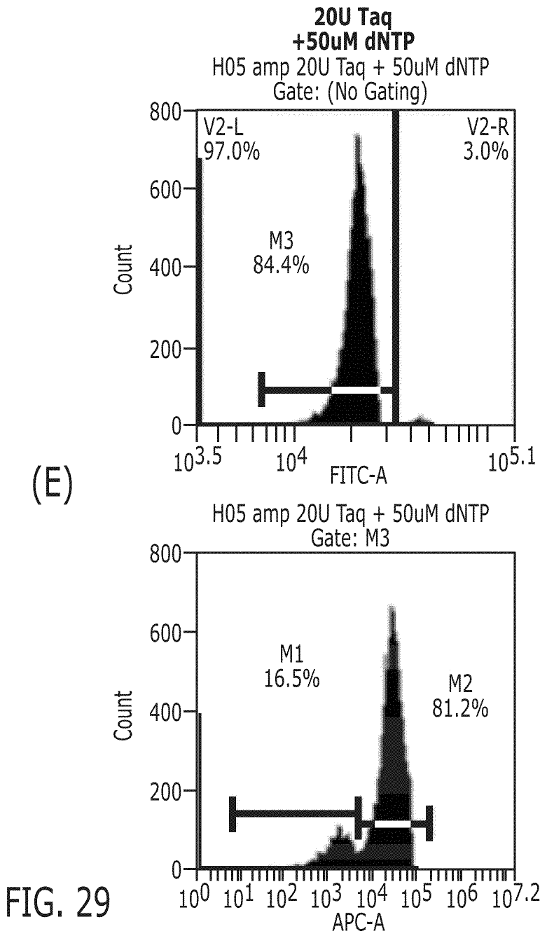

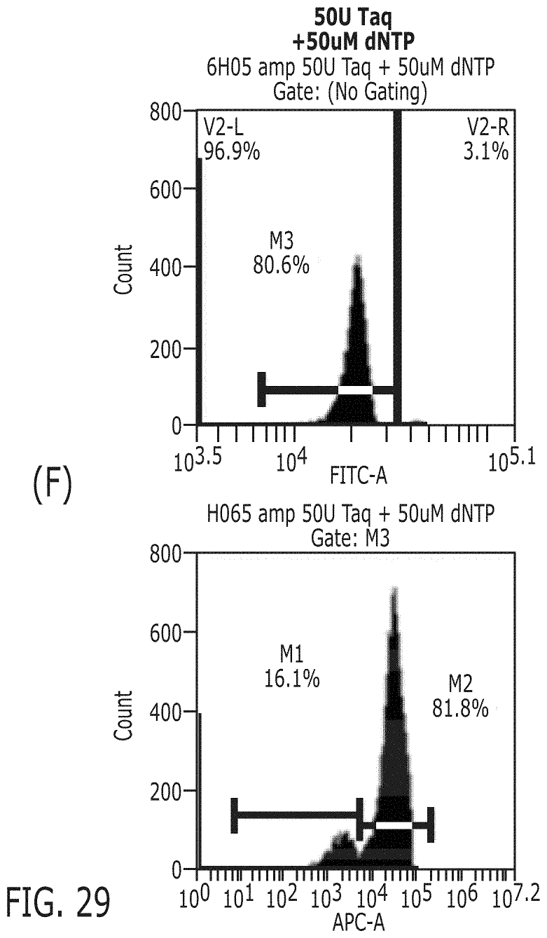

FIG. 28 illustrates additional example data of pre-enrichment efficiencies (% amp) of commercially available bead samples (H01 to H06) with templates ligated thereto with or without Taq and nucleotides. H01 has no nucleotides or Taq added (“Std PE amp”), which is a double-negative control; H02 has 50 micromolar nucleotides (“50 uM dNTP”) but no Taq as a negative control; H03 has 10 units of Taq (“10 U Taq”) with no nucleotides as a negative control; H04 has 10 units Taq and 50 micromolar of dNTPs added; H05 has 20 units Taq and 50 micromolar of dNTPs added; H06 has 50 units of Taq and 50 micromolar of dNTPs. As indicated in the pre-enrichment efficiency (% amp), the control groups (H01-H03) all have a pre-enrichment efficiency of 72-77%, whereas the groups with Taq and dNTPs have an increased pre-enrichment efficiency of 81%. FIG. 29 shows the flow cytometry histograms of the experimental groups shown in FIG. 28. Altogether, these results indicate that the addition of Taq polymerase and nucleotides may improve ligation efficiency of template nucleic acid molecules to supports.

### Example 15

Template nucleic acid molecules may be attached to supports (e.g., beads) using an intermediary molecule, such as a splint oligonucleotide and/or a bridge molecule. An example workflow of using a splint and bridge molecule for attachment of a template nucleic acid molecule to a support is shown in FIG. 22E, and an example of such an annealed molecule comprising the support, bridge molecule, and splint molecule is shown in FIG. 30.

In some embodiments, the timing of the addition of splint molecules (and bridge molecules) to the reaction mix affects the speed of the reaction. In some embodiments, addition of the splint and bridge molecules during ligation will slow down the reaction (e.g., by slowing the reaction kinetics of template nucleic acid molecules attaching to supports). In some embodiments, the splint and bridge molecules are added to the reaction mix prior to ligation. This permits normalization of the ratio of template nucleic acid molecules to supports prior to the ligation reaction.

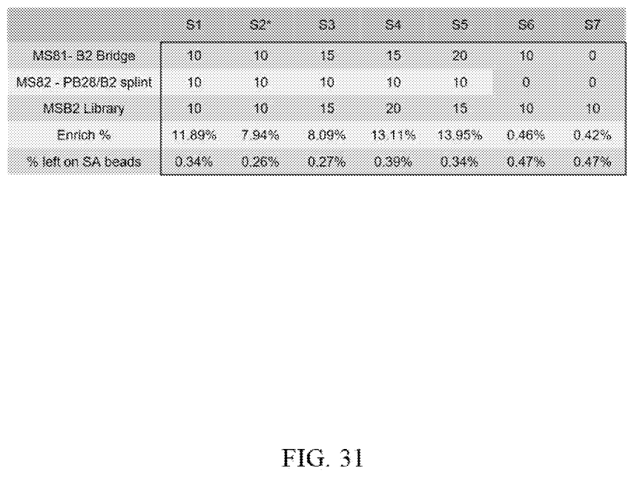

In an example, an excess of a template nucleic acid molecules may be provided as well as an excess of beads (e.g., at a 1:10 ratio of template nucleic acid molecules to beads). The bridge molecule and splint molecule, as shown in FIG. 30 may be provided at a known concentration that is lower than or approximately the same as the concentration of the template nucleic acid molecules. FIG. 31 shows an example of relative concentrations of a bridge molecule (“MS81-B2 Bridge”), a splint molecule (“MS82-PB28/B2 splint”), and the template nucleic acid molecules (“MSB2 library”) in different experiments (S1-S7). The bridge and splint molecules may be provided at a concentration equivalent to or less than the concentration of the template nucleic acid molecules. The template nucleic acid molecules, splint molecules, and bridge molecules are hybridized to a support (e.g., bead) and optionally, ligated, to generate a plurality of support-template nucleic acid complexes. The support-template nucleic acid complexes are then pre-enriched (e.g., using a capture entity that can capture a sequence or capture moiety such as biotin), and the pre-enrichment percentage is calculated. As illustrated in FIG. 31, the enrichment percentages are as expected (˜10%). The negative controls (S6 and S7) comprising either no splint (S6) or no splint and no bridge (S7) indicate a near-zero enrichment percentage, as expected. These results suggest that in the absence of the splint molecule, no template nucleic acid molecules ligate to the bead and thus no enrichment occurs, and that controlling the relative concentration of a splint and bridge molecule relative to the template nucleic acid molecules can determine the support-template nucleic acid molecule attachment ratio.

Such a process (e.g., controlling the relative ratios of the splint molecule, bridge molecule, and template nucleic acid molecules, and optionally, the support concentration), may be useful or advantageous in controlling the support-template nucleic acid molecule attachment, and thus the pre-enrichment ratio. Accordingly, in instances where the template nucleic acid molecule concentration is unknown (e.g., following amplification and cleavage of a cleavable moiety to render the molecule ligatable to the support), the attachment efficiency of the template nucleic acid molecule to the support may be controlled by determining the ratio of splints and bridges that are provided. In such an example, it may not be necessary to quantitate the template nucleic acid molecules present in a sample to enable efficient support attachment.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-161. (canceled)

162. A method comprising:
(a) providing a solution comprising:
(i) a first support and a second support, wherein said first support comprises a first primer of a plurality of first primers and said second support comprises a second primer of a plurality of second primers, wherein said first primer and said second primer comprise different nucleic acid sequences;
(ii) a first nucleic acid molecule and a second nucleic acid molecule, wherein said first nucleic acid molecule comprises a first adapter sequence configured to hybridize to said first primer, wherein said second nucleic acid molecule comprises a second adapter sequence configured to hybridize to said second primer; and
(iii) one or more reagents;
(b) (i) using said first nucleic acid molecule and said one or more reagents to generate a plurality of first amplification products, wherein at least a subset of said plurality of first amplification products are attached to said first support via said plurality of first primers; and
(ii) using said second nucleic acid molecule and said one or more reagents to generate a plurality of second amplification products, wherein at least a subset of said plurality of second amplification products are attached to said second support via said plurality of second primers; and
(c) assaying
(i) first amplification products of said subset of said plurality of first amplification products attached to said first support, or derivatives thereof, and
(ii) second amplification products of said subset of said plurality of second amplification products attached to said second support, or derivatives thereof, to identify a first sequence of said first nucleic acid molecule and a second sequence of said second nucleic acid molecule.

163. The method of claim 162, wherein (b) is performed within a bulk solution.

164. The method of claim 162, wherein, prior to (b), said first support, said second support, said first nucleic acid molecule, and said second nucleic acid molecule are included within a bulk solution.

165. The method of claim 162, wherein said first nucleic acid molecule is immobilized to said first support via said first primer and said second nucleic acid molecule is immobilized to said second support via said second primer, wherein said method further comprises, prior to (b), isolating said first support and said second support from supports that do not have immobilized thereon any nucleic acid molecule of a plurality of template nucleic acid molecules, wherein said plurality of template nucleic acid molecules comprises said first nucleic acid molecule and said second nucleic acid molecule.

166. The method of claim 162, wherein said first primer comprises a first extended primer region that is absent from other first primers in said plurality of first primers and said second primer comprises a second extended primer region that is absent from other second primers in said plurality of second primers, and wherein said first nucleic acid molecule is immobilized to said first support via said first extended primer region and said second nucleic acid molecule is immobilized to said second support via said second extended primer region.

167. The method of claim 162, wherein each first primer of said plurality of first primers are identical.

168. The method of claim 162, further comprising, prior to (b), generating a first partition comprising said first support and said first nucleic acid molecule and a second partition comprising said second support and said second nucleic acid molecule.

169. The method of claim 168, wherein said plurality of first amplification products are generated within said first partition and said plurality of second amplification products are generated within said second partition.

170. The method of claim 162, wherein said first support and said second support are a first bead and a second bead, respectively.

171. The method of claim 162, wherein said first support and said second support are immobilized to a substrate.

172. The method of claim 171, wherein said substrate comprises a substantially planar array.

173. The method of claim 171, wherein said substrate comprises a plurality of individually addressable locations.

174. The method of claim 162, wherein said first nucleic acid molecule comprises a third adapter sequence disposed at an end distal to said first adapter sequence and said second nucleic acid molecule comprises a fourth adapter sequence disposed at an end distal to said second adapter sequence.

175. The method of claim 174, wherein said third adapter sequence and said fourth adapter sequence comprise identical nucleic acid sequences, wherein said one or more reagents comprise a plurality of additional primers, and wherein an additional primer of said plurality of additional primers is configured to hybridize to said third adapter sequence and said fourth adapter sequence.

176. The method of claim 174, wherein said third adapter sequence and said fourth adapter sequence comprise different nucleic acid sequences.

177. The method of claim 176, wherein said one or more reagents comprise a plurality of additional primers, and wherein said plurality of additional primers comprises a first additional primer configured to hybridize to said third adapter sequence and a second additional primer configured to hybridize to said fourth adapter sequence.

178. The method of claim 162, wherein (b) comprises performing polymerase chain reaction (PCR).

179. The method of claim 162, wherein (b) comprises performing recombinase polymerase amplification (RPA).

180. The method of claim 162, wherein said one or more reagents comprises a binding protein.

181. The method of claim 162, wherein said one or more reagents comprises a crowding agent.


SEQUENCE LISTING (6 sequences)

SEQ ID NO: 1 - DNA, 14 bp, synthetic construct
  source 1..14: mol_type = other DNA
  modified_base 14: mod_base = OTHER; note = This nucleotide is linked at the 3' end to a nucleotide having an 5' end of CACCTCC and a C3 linker/spacer sequence of undefined length
acggtttctccatg

SEQ ID NO: 2 - DNA, 80 bp, synthetic construct
  source 1..80: mol_type = other DNA
cctatcccctgtgtgccttggcagtctcccgcctcgctgtcacctgctgctgatttctctctctatgggcagtcggtgat

SEQ ID NO: 3 - DNA, 73 bp, synthetic construct
  source 1..73: mol_type = other DNA
  modified_base 28: mod_base = OTHER; note = uracil
  modified_base 39: mod_base = OTHER; note = uracil
  modified_base 58: mod_base = OTHER; note = uracil
  modified_base 72: mod_base = OTHER; note = internucleotide phosphorothioate linkage
  modified_base 73: mod_base = OTHER; note = 3' Hexanediol nucleotide
atcaccgactgcccatagagagagaaatcagcagcaggtgacagcgaggcgggagactgccaaggcacacagg

SEQ ID NO: 4 - DNA, 30 bp, synthetic construct
  source 1..30: mol_type = other DNA
  modified_base 1: mod_base = OTHER; note = 5'-phosphorylated nucleotide
ccgcctcgctgtcacctgctgctgatttct

SEQ ID NO: 5 - DNA, 30 bp, synthetic construct
  source 1..30: mol_type = other DNA
  modified_base 1: mod_base = OTHER; note = 5'-phosphorylated nucleotide
  modified_base 29: mod_base = OTHER; note = internucleotide phosphorothioate linkage
  modified_base 30: mod_base = OTHER; note = 3' Hexanediol nucleotide
gcgaggcgggagactgccaaggcacacagg

SEQ ID NO: 6 - DNA, 37 bp, synthetic construct
  source 1..37: mol_type = other DNA
  modified_base 32: mod_base = OTHER; note = 1',2'-Dideoxyribose nucleotide
  modified_base 35: mod_base = OTHER; note = Fluorescein modified nucleotide
ggaaggccgctgacagtttttctgtcagcggycttcc